(19) United States
(12) Patent Application Publication
(10) Pub. No.: US 2022/0121113 A1
(43) Pub. Date: Apr. 21, 2022

(54) OXIME ESTER PHOTOINITIATORS HAVING A SPECIAL AROYL CHROMOPHORE

(71) Applicant: BASF SE, Ludwigshafen am Rhein (DE)

(72) Inventors: Kazuhiko KUNIMOTO, Amagasaki (JP); Hisatoshi KURA, Amagasaki (JP); Kaori SAMESHIMA, Amagasaki (JP); Takeo FUKUZUMI, Amagasaki (JP); Yuki MATSUOKA, Amagasaki (JP)

(21) Appl. No.: 17/424,585

(22) PCT Filed: Jan. 1, 2020
(86) PCT No.: PCT/EP2020/051328

§ 371 Date: Jul. 21, 2021

(30) Foreign Application Priority Data
Jan. 23, 2019 (EP) 19153177.1

(51) Int. Cl. G03F 7/00; G03F 7/004; G03F 7/038; G03F 7/20; G03F 7/32

(52) U.S. Cl. CPC G03F 7/0007; G03F 7/0045; G03F 7/322; G03F 7/2012; G03F 7/0388

(57) ABSTRACT

Disclosed are α-keto oxime ester carbazole derivatives with a specific aroyl chromophore useful as a photo initiator, as well as photopolymerizable compositions comprising said photo initiator and ethylenically unsaturated compounds. The photopolymerizable compositions are useful, for example, in photoresist formulations for display applications, e.g. liquid crystal display (LCD), organic light emitting diode (OLED) and touch panel.

The present invention relates to novel oxime ester compounds with a specific aroyl chromophore useful as a photoinitiator, and to photopolymerizable compositions comprising said compounds and ethylenically unsaturated compounds.

Photosensitive compositions are prepared by adding a photoinitiator to a polymerizable compound having an ethylenically unsaturated bond. Such photosensitive compositions can be polymerized and cured upon irradiation with polychromatic light with wavelengths of 365 nm, 405 nm, and 436 nm and are thus used in photocurable inks, photosensitive printing plates, various types of photoresists, etc. Photosensitive compositions sensitive to short-wavelength light sources can be micro-printed. Thus, photopolymerization initiators are particularly needed that have high sensitivity to short-wavelength light sources, particularly, 365 nm light sources. Many oxime ester compounds are used as highly sensitive photoinitiators. Numerous characteristics of oxime ester compounds are described in several patent publications and some commercial products for oxime ester compounds are known. Most of such oxime ester photoinitiators are currently applied to photoresists in the field of LCDs. Commercially available products for oxime ester photoinitiators are divided into α-ketoxime ester compounds and oxime ester compounds. α.-ketoxime ester photoinitiators are used in color photoresists, mainly red, green, and blue photoresists. Oxime ester compounds may be decomposed when irradiated with UV light. This photodecomposition changes the color of resist films. In contrast, α-ketoxime ester photoinitiators do not tend to discolor when exposed to UV light, causing no change in the color coordinates of color resists. For this reason, α-ketoxime ester compounds are mainly used in color photoresists. However, currently commercially available α-ketoxime ester photopolymerization initiators suffer from the problem of low sensitivity. Under these circumstances, a need exists for highly sensitive α-ketoxime ester photopolymerization initiators. As photoinitiators used for the photosensitive compositions, α-ketoxime carbazole derivatives having aroyl and/or heteroaroyl group are known in the following arts.

WO2002100903 discloses α-ketoxime ester derivatives. These compounds have hetero aryl carbonyl or aryl carbonyl group attached to carbazole moiety. JP2009221334 discloses oxime ester photoinitiators of the high photosensitivity having heteroaromatic chromophore attached at least one polymerizable substituent. WO2009131189 discloses ketoxime derivatives including α-ketoxime carbazole having aroyl and/or heteroaroyl group. These compounds have at oxime moiety, for example, substituted and/or unsubstituted C₁-C₂₀alkylC₂-C₁₂alkoxycarbonyl, C₁-C₂₀alklC₁-C₁₂alkylthio group. US20100104976 discloses oxime ester derivatives including α-ketoxime carbazoles. These compounds had alkyl group at oxime moiety which is substituted by ArS. JP2011074042 discloses α-ketoxime carbazoles derivatives having aroyl group. These compounds have electron withdrawing group attached to N-substituent, and have alkyl, alkenyl or alkynyl group at oxime moiety which is substitute by ArS. WO2012045736 discloses broad scope of benzocarbazole derivatives as high photosensitive photoinitiators. These compounds include α-ketoxime benzocarbazole derivatives having aroyl group and heteroaroyl group as sensitizer unit. KR101225695 discloses α-ketoxime derivatives as high photosensitive photoinitiator. These compounds include carbazole α-ketoxime chromophore having aroyl and/or heteroaroyl group, and phenyl substituted by methyl group at oxime moiety. CN103130919 discloses carbazole ketoxime ester as high photosensitivity photoinitiators. These compounds have alkyl group substituted by cycloalkyl group at the oxime position, and aroyl group having 0, S N-atom containing substituent or O, S-containing heteroaroyl group as sensitizer unit. CN103204960 discloses α-ketoxime carbazole photoinitiators having aroyl group such as tolyl group as sensitizer unit. These compounds have morpholine methyl group at the oxime moiety. JP2013142087 includes α-ketoxime carbaxole derivatives having aroyl and/or heteroaroyl group, and nitrobenzene group at the oxime moiety. CN103998422 discloses cyclopentandione oxime ester derivatives. These compounds include α-ketoxime carbazole derivative such as [(E)-[9-(2-methylbenzoyl)-3-oxo-1,6-dihydrocyclopenta[c]carbazol-2-ylidene] amino] acetate. KR1457172 includes α-ketoxime carbaxole derivatives having aroyl and/or heteroaroyl group, and alkyl and/or aryl group at the oxime moiety. KR2017009794 discloses broad scope of oxime esters which include α-keto oxime carbaxole derivatives having electron withdrawing group such as aroyl, heteroaroyl, nitro, cyano group. WO2017131378 includes α-ketoxime carbazoles derivatives having aroyl group or nitro group on carbazole chromophore. These compounds also have phosphate group attached to N-substituent. WO17209449 includes α-keto oxime carbazole derivatives having aroyl group. These compounds have phosphate group attached to alkyl group at oxime moiety and at least one polymerizable substituent. JP2009179619 discloses α-keto oxime carbazole derivatives having aroyl group at the base of oxime group.

The coloration alkaline developable photosensitive resin composition containing a coloring agent used for color filters is particularly required high sensitive, and it is necessary to make a photoinitiator in the resist high density because absorption of colorant in the ultraviolet region makes curing by polymerization insufficient. However, the highly-concentrated photoinitiator causes residual outbreak by the developing-related aggravation or the pollution of photomask and/or the heating furnace by the sublimation. In addition, in the coloration alkali developable photosensitive resin composition containing a coloring agent used for color filters, the photoinitiator to be used is demanded to show low discoloration in a visible light region after post-baking. If the discoloration after post-baking in the visible light region is high, the problem such as the deterioration of the color property occurs.

Photoresist compounds undergo photocuring to form patterns when irradiated with UV. Highly sensitive photoinitiators with high photoreactivity and easy-to-prepare and easy-to-handle photoinitiators with high solubility are required to shorten the processing time of photocuring. For example, when photoresist compounds are applied to color resists, resists are needed which include pigments dispersed by advanced techniques in order to achieve high color quality characteristics. A higher pigment content tends to make the curing of a color resist more difficult. Thus, there is a need for initiators that have higher photosensitivity than general-use initiators. Such photoinitiators are required to meet stringent requirements in terms of industrially relevant characteristics, such as high solubility in organic solvents and good thermal and storage stability. It is an object of the present invention to provide an α-ketoxime ester compound or an oxime ester compound as a highly sensitive photoinitiator that has a UV absorbance peak at a wavelength close to 365 nm to 410 nm while possessing excellent characteristics in terms of developability, adhesiveness, and alkali resistance. Surprisingly, it was found that compounds of α-ketoxime carbazole derivatives having specific aroyl group showed not only excellent performance in aspects of photosensitivity, but also exhibits obviously improved low discoloration compared to similar photoinitiators.

Accordingly, the present invention provides compounds of the formula I

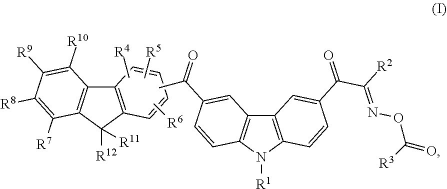

wherein

R¹ is C₁-C₂₀alkyl which is unsubstituted or substituted by one or more halogen, OR¹⁴, SR¹⁵, COOR¹⁴, CONR¹⁶R¹⁷, NR¹⁶R¹⁷, PO(OCₖH₂ₖ₊₁)₂; or by

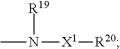

R¹ is C₂-C₂₀alkyl which is interrupted by one or more O, CO, S, SO, SO₂, C(O)O, OC(O), phenylene, naphthylene or NR¹⁸, wherein the interrupted C₂-C₂₀alkyl is unsubstituted or substituted by one or more halogen, OR¹⁴, SR¹⁵, COOR¹⁴, CONR¹⁶R¹⁷, NR¹⁶R¹⁷; R¹ is C₂-C₁₂alkenyl which is uninterrupted or is interrupted by one or more O, CO, S, SO, SO₂, C(O)O, OC(O), phenylene, naphthylene or NR¹⁸, wherein the uninterrupted or interrupted C₂-C₁₂alkenyl are unsubstituted or are substituted by one or more halogen, OR¹⁴, SR¹⁵, COOR¹⁴, CONR¹⁶R¹⁷, NR¹⁶R¹⁷;

R¹ is C₄-C₈cycloalkenyl, C₂-C₁₂alkinyl, or C₃-C₁₀cycloalkyl which is uninterrupted or is interrupted by one or more O, CO, S, SO, SO₂, C(O)O, OC(O), phenylene, naphthylene or NR¹⁸; or R¹ is C₆-C₂₀aryl or C₃-C₂₀heteroaryl each of which is unsubstituted or substituted by one or more phenyl, halogen, C₁-C₄haloalkyl, CN, NO₂,

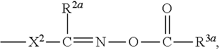

COR^{3a}, OR¹⁴, SR¹⁵, NR¹⁶R¹⁷, or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷, or each of which is substituted by C₃-C₁₀cycloalkyl or by C₃-C₁₀cycloalkyl which is interrupted by one or more O, S, CO, or NR¹⁸; R² is hydrogen, CN, COR^{3a}, COOR¹⁴, CONR¹⁶R¹⁷, NO₂, PO(OR^{3a})₂ or S(O)ₘR^{3a}; or R² is C₁-C₂₀alkyl which is unsubstituted or substituted by one or more substituents selected from the group consisting of halogen, OR¹⁴, SR¹⁵, NR¹⁶R¹⁷, CN, COOR¹⁴, CONR¹⁶R¹⁷, PO(OR^{3a})₂, S(O)ₘR^{3a},

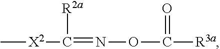

C₃-C₈cycloalkyl which is uninterrupted or interrupted by one or more O, S, CO or NR¹⁶,

C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵, NR¹⁶R¹⁷, PO(OR^{3a})₂ or S(O)ₘR^{3a}; or

R² is C₂-C₂₀alkyl interrupted by one or more O, S, NR¹⁶, CO, SO or SO₂, which interrupted C₂-C₂₀alkyl is unsubstituted or substituted by one or more C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵ or NR¹⁶R¹⁷; or

R² is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR¹⁶ or COOR¹⁴; or

R² is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR¹⁴, SR¹⁵, NR¹⁶R¹⁷, COOR¹⁴, COR^{3a} CONR¹⁶R¹⁷, PO(OR^{3a})₂, S(O)ₘR^{3a},

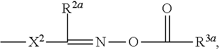

C₁-C₂₀alkyl which is unsubstituted or substituted by one or more halogen, COOR^{14a}, CONR¹⁶R¹⁷, phenyl, C₃-C₈cycloalkyl, C₃-C₂₀heteroaryl, OR¹⁴, SR¹⁵ or NR¹⁶R¹⁷,

C₂-C₂₀alkyl which is interrupted by one or more O, S or NR¹⁶, phenyl, naphthyl, benzoyl and naphthoyl, which phenyl, naphthyl, benzoyl or naphthoyl is unsubstituted or substituted by one or more OR¹⁴, SR¹⁵ or NR¹⁶R¹⁷;

R^{2a} is hydrogen, CN, COR^{3a}, COOR^{14a}, CONR^{16a}R^{17a}, NO₂, PO(OR^{3a})₂ or S(O)ₘR^{3a}; or

R^{2a} is C₁-C₂₀alkyl which is unsubstituted or substituted by one or more substituents selected from the group consisting of

halogen, OR^{14a}, SR^{15a}, NR^{16a}R^{17a}, CN, COOR^{14a}, CONR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or

R^{2a} is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or

R^{2a} is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or

R^{2a} is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a}, C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a}; C₂-C₂₀alkyl which is interrupted by one or more O, S or NR^{16a}, phenyl, naphthyl, benzoyl and naphthoyl, each of which phenyl, naphthyl, benzoyl or naphthoyl is unsubstituted or substituted by one or more OR^{14a}, SR^{15a} or NR^{16a}R^{17a}; R³ is hydrogen, C₁-C₂₀alkyl which is unsubstituted or substituted by one or more substituents selected from the group consisting of halogen, OR¹⁴, SR¹⁵, NR¹⁶R¹⁷, CN, COOR¹⁴, CONR¹⁶R¹⁷, C₃-C₈cycloalkyl, C₃-C₈cycloalkyl which is interrupted by one or more O, S, CO or NR¹⁶, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵ or NR¹⁶R¹⁷; or

R³ is C₂-C₂₀alkyl interrupted by one or more O, S, NR¹⁶, CO, SO or SO₂, which interrupted C₂-C₂₀alkyl is unsubstituted or substituted by one or more C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵ or NR¹⁶R¹⁷; or

R³ is C₂-C₁₂alkenyl or C₃-C₂₀cycloalkyl, each of which is uninterrupted or interrupted by one or more O, S, CO, NR¹⁶ or COOR¹⁴; or

R³ is C₆-C₂₀aryl or C₃-C₂₀heteroaryl, each of which is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, COR^{3a} or SO₂—R^{3a}; or

R³ is C₁-C₂₀alkoxy, which is unsubstituted or substituted by one or more C₁-C₁₀alkyl, C₁-C₄haloalkyl, halogen, phenyl, C₁-C₂₀alkylphenyl or C₁-C₈alkoxyphenyl; or

R³ is C₂-C₂₀alkoxy, which is interrupted by one or more O, S, NR¹⁶, CO, SO or SO₂; or

R³ is C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, (CO)R^{3a} or SO₂R^{3a}; R^{3a} is hydrogen, C₁-C₂₀alkyl, C₁-C₄haloalkyl which is unsubstituted or substituted by one or more substituents selected from the group consisting of halogen, OR^{14a}, SR^{15a}, NR^{16a}R^{17a}, CN, COOR^{14a}, CONR^{16a}R^{17a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a} or NR^{16a}R^{17a}; or

R^{3a} is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)—(C₁-C₈alkyl), COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or

R^{3a} is C₂-C₁₂alkenyl or C₃-C₂₀cycloalkyl, each of which is uninterrupted or interrupted by one or more O, S, CO, NR^{16a} or COOR^{14a}; or

R^{3a} is C₆-C₂₀aryl or C₃-C₂₀heteroaryl, which C₆-C₂₀aryl or C₃-C₂₀heteroaryl is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR^{14a}, SR^{15a}, NR^{16a}R^{17a}, COOR^{14a}, (CO)—(C₁-C₈alkyl), benzoyl or SO₂—(C₁-C₄haloalkyl); or

R^{3a} is C₁-C₂₀alkoxy, which is unsubstituted or substituted by one or more C₁-C₁₀alkyl, C₁-C₄haloalkyl, halogen, phenyl, C₁-C₂₀alkylphenyl or C₁-C₈alkoxyphenyl; or

R^{3a} is C₂-C₂₀alkoxy, which is interrupted by one or more O, S, NR^{16a}, CO, SO or SO₂; or

R^{3a} is C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR^{14a}, SR^{15a}, NR^{16a}R^{17a}, COOR^{14a}, (CO)(C₁-C₈alkyl), SO₂—(C₁-C₄haloalkyl), benzoyl, or benzoyl or

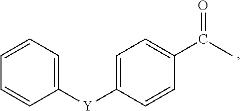

which benzoyl or

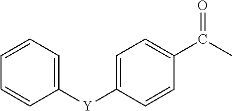

is unsubstituted or substituted by

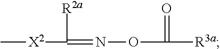

R⁴, R⁵, R⁶, R⁷, R⁸, R⁹ and R¹⁰ independently of each other are hydrogen, halogen, C₁-C₂₀alkyl, C₁-C₂₀haloalkyl, C₆-C₂₀aryl, C₁-C₂₀alkoxy, C₆-C₂₀arylC₁-C₂₀alkyl, C₃-C₁₀cycloalkyl, amino, CN, NO₂, O(CO)C₁-C₂₀alkyl, O(CO)C₆-C₁₀aryl,

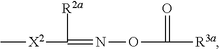

COR¹³, COOR¹⁴, or

R⁴ and R⁵ or R⁵ and R⁶, R⁷ and R⁸, R⁸ and R⁹, and R⁹ and R¹⁰ are

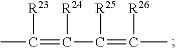

R¹¹ and R¹² independently of each other are hydrogen, halogen, C₁-C₃₀alkyl, C₆-C₂₀aryl, C₁-C₂₀alkoxy, C₆-C₂₀arylC₁-C₂₀alkyl, hydroxyC₁-C₂₀alky, hydroxyC₁-C₂₀alkoxyC₁-C₂₀alkyl, C₃-C₂₀cycloalkyl, C₃-C₂₀heterocycloalkyl, C₃-C₂₀cycloalkylC₁-C₂₀alkyl, C₂-C₃₀alkenyl, C₂-C₃₀alkynyl; or

R¹¹ and R¹² are connected to each other to form a C₂-C₅alkylene ring, C₂-C₅alkenylene ring, or C₂-C₅alkynylene ring;

R¹³ is C₆-C₂₀aryl or C₃-C₂₀heteroaryl each of which is unsubstituted or substituted by one or more phenyl, halogen, C₁-C₄haloalkyl, CN, NO₂, OR¹⁴, SR¹⁵, NR¹⁶R¹⁷, or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷;

R¹⁴ is hydrogen, COR^{3a}, COOR^{14a}, CONR¹⁶R¹⁷, C₁-C₂₀alkyl which is unsubstituted or substituted by one or more substituents selected from the group consisting of halogen, OR^{14a}, SR^{15a}, NR^{16a}R^{17a}, CN, COOR^{14a}, CONR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or

R¹⁴ is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or

R¹⁴ is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or

R¹⁴ is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a},

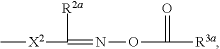

C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a}, C₂-C₂₀alkyl which is interrupted by one or more O, S or NR^{16a};

phenyl, naphthyl, benzoyl and naphthoyl, which phenyl, naphthyl, benzoyl or naphthoyl is unsubstituted or substituted by one or more OR^{14a}, SR^{15a} or NR^{16a}R^{17a}; or

R¹⁴ together with one of the carbon atoms of R₂ or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a} or NR^{16a}R^{17a}, COR^{3a}, NO₂, halogen, C₁-C₄haloalkyl, CN, phenyl, C₁-C₂₀alkylphenyl, C₁-C₈alkoxyphenyl,

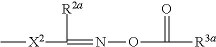

or C₃-C₂₀cycloalkyl which is uninterrupted or interrupted by one or more O, S, CO or NR^{16a};

R^{14a} is hydrogen, (CO)O(C₁-C₈alkyl) or CON(C₁-C₈alkyl)₂; or

R^{14a} is C₁-C₂₀alkyl which is unsubstituted or substituted by one or more substituents selected from the group consisting of halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl and C₃-C₈cycloalkyl which is interrupted by one or more O; or

R^{14a} is C₂-C₂₀alkyl interrupted by one or more O, S, N(C₁-C₈alkyl), CO, SO or SO₂, which interrupted C₂-C₂₀alkyl is unsubstituted or substituted by one or more C₃-C₈cycloalkyl, OH, SH, O(CO)(C₁-C₈alkyl), (CO)O(C₁-C₈alkyl), (CO)N(C₁-C₈alkyl)₂, C₆-C₂₀aryl, C₃-C₂₀het¬eroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or

R^{14a} is C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or

R^{14a} is C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₃alkyloxy, phenoxy, C₁-C₈alkylsulfanyl, phenylsulfanyl, N(C₁-C₈alkyl)2, diphenylamino, (CO)O(C₁-C₈alkyl), (CO)C₁-C₈alkyl or (CO)N(C₁-C₈)₂, phenyl or benzoyl; or

R^{14a} is C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino;

R¹⁵ is hydrogen, C₁-C₂₀alkyl which is unsubstituted or substituted by one or more substituents selected from the group consisting of halogen, OR^{14a}, SR^{15a} or NR^{16a}R^{17a}, CN, COOR^{14a}, CONR^{16a}R^{17a}, PO(OR^{3a})₂, S(O)ₘR^{3a},

C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or

R¹⁵ is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a}, or NR^{16a}R^{17a}; or

R¹⁵ is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or

R¹⁵ is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a},

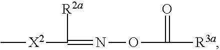

C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a}, C₂-C₂₀alkyl which is interrupted by one or more O, S or NR^{16a},

phenyl, naphthyl, benzoyl and naphthoyl, which phenyl, naphthyl, benzoyl or naphthoyl is unsubstituted or substituted by OR^{14a}, SR^{15a} or NR^{16a}R^{17a}; or

R¹⁵ together with one of the carbon atoms of R₂ or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄-haloalkyl, CN, phenyl, C₁-C₂₀alkylphenyl, C₁-C₈alkoxyphenyl,

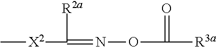

or C₃-C₂₀cycloalkyl which is uninterrupted or interrupted by one or more O, S, CO or NR^{16a};

R^{15a} is hydrogen, C₁-C₂₀alkyl which is unsubstituted or substituted by one or more substituents selected from the group consisting of halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl and C₃-C₈cycloalkyl which is interrupted by one or more O; or

R^{15a} is C₂-C₂₀alkyl interrupted by one or more O, S, N(C₁-C₈alkyl), CO, SO or SO₂, which interrupted C₂-C₂₀alkyl is unsubstituted or substituted by one or more C₃-C₈cycloalkyl, OH, SH, O(CO)(C₁-C₈alkyl), (CO)O(C₁-C₈alkyl), (CO)N(C₁-C₈alkyl)2, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or

R^{15a} is C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or

R^{15a} is C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₈alkyloxy, phenoxy, C₁-C₈alkylsulfanyl, phenylsulfanyl, N(C₁-C₈alkyl)₂, diphenylamino, (CO)O(C₁-C₈alkyl), (CO)C₁-C₈alkyl, (CO)N(C₁-C₈alkyl)₂, phenyl or benzoyl; or

R^{15a} is C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino;

R¹⁶ and R¹⁷ independently of each other are hydrogen, S(O)ₘR^{3a}, O(CO)R^{3a}, COR^{3a} or CONR^{16a}R^{17a}; or

R¹⁶ and R¹⁷ independently of each other are C₁-C₂₀alkyl which is unsubstituted or substituted by one or more substituents selected from the group consisting of halogen, OR^{14a}, SR^{15a}, NR^{16a}R^{17a}, CN, COOR^{14a}, COR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or

R¹⁶ and R¹⁷ independently of each other are C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, COR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or

R¹⁶ and R¹⁷ independently of each other are C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or

R¹⁶ and R¹⁷ independently of each other are C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a}, CONR^{16a}R^{17a}, PO(OR^{3a})₂, S(O)ₘR^{3a},

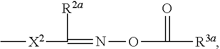

C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a}, or NR^{16a}R^{17a}, C₂-C₂₀alkyl which is interrupted by one or more O, S or NR^{16a}, phenyl, naphthyl, benzoyl and naphthoyl, which phenyl, naphthyl, benzoyl or naphthoyl is unsubstituted or substituted by OR^{14a}, SR^{15a}, or NR^{16a}R^{17a}; or

R¹⁶ and R¹⁷ independently of each other are C₁-C₂₀alkoxy, which is unsubstituted or substituted by one or more halogen, phenyl, C₁-C₈alkylphenyl or C₁-C₈alkoxyphenyl; or

R¹⁶ and R¹⁷ independently of each other are C₂-C₂₀alkoxy, which is interrupted by one or more O, S, NR^{16a}, CO, SO or SO₂; or

R¹⁶ and R¹⁷ independently of each other are C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₈alkyl, C₁-C₄haloalkyl, phenyl, C₁-C₈alkylphenyl, C₁-C₈alkoxyphenyl, CN, NO₂, OR^{14a}, SR^{15a}, NR^{16a}R^{17a}, COOR^{14a}, COR^{3a} or SO₂R^{3a}; or

R¹⁶ together with one of the carbon atoms of R² or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄haloalkyl, CN, phenyl, C₁-C₂₀alkylphenyl, C₁-C₈alkoxyphenyl,

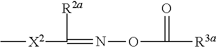

or C₃-C₂₀cycloalkyl which is uninterrupted or interrupted by one or more O, S, CO or NR^{16a}; or

R¹⁶ and R¹⁷ together with the N-atom to which they are attached form a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄haloalkyl, CN, phenyl,

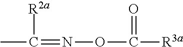

or C₃-C₂₀cycloalkyl which is uninterrupted or interrupted by one or more O, S, CO or NR^{16a};

R^{16a} and R^{17a} independently of each other are hydrogen, C₁-C₂₀alkyl, C₁-C₄haloalkyl, S(O)ₘ(C₁-C₈alkyl), O(CO)(C₁-C₈alkyl), (CO)(C₁-C₈alkyl), (CO)O(C₁-C₈alkyl) or CON(C₁-C₈alkyl)₂; or

R^{16a} and R^{17a} independently of each other are C₁-C₂₀alkyl substituted by one or more halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl, C₃-C₈cycloalkyl or C₃-C₈cycloalkyl which is interrupted by one or more O; or

R^{16a} and R^{17a} independently of each other are C₂-C₂₀alkyl interrupted by one or more O, S, N(C₁-C₈alkyl), CO, SO or SO₂, which interrupted C₂-C₂₀alkyl is unsubstituted or substituted by C₃-C₈cycloalkyl, OH, SH, O(CO)(C₁-C₈alkyl), (CO)O(C₁-C₈alkyl), (CO)N(C₁-C₈alkyl)₂, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or

R^{16a} and R^{17a} independently of each other are C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or

R^{16a} and R^{17a} independently of each other are C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₃alkyloxy, phenoxy, C₁-C₈alkylsulfanyl, phenylsulfanyl, N(C₁-C₈alkyl)₂, diphenylamino, (CO)O(C₁-C₈alkyl), (CO)C₁-C₈alkyl, (CO)N(C₁-C₈alkyl)₂, phenyl or benzoyl; or

R^{16a} and R^{17a} independently of each other are C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino; or

R^{16a} and R^{17a} independently of each other are C₁-C₂₀alkoxy, which is unsubstituted or substituted by one or more halogen, phenyl, C₁-C₈alkylphenyl or C₁-C₈alkoxyphenyl; or

R^{16a} and R^{17a} independently of each other are C₂-C₂₀alkoxy, which is interrupted by one or more O, S, N(C₁-C₈alkyl), CO, SO or SO₂; or

R^{16a} and R^{17a} independently of each other are C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₈alkyl, C₁-C₄haloalkyl, phenyl, C₁-C₈alkylphenyl, C₁-C₈alkoxyphenyl, CN, NO₂, C₁-C₈alkoxy, C₁-C₈alkylsulfanyl, N(C₁-C₈alkyl)₂, CO(OC₁-C₈alkyl), (CO)(C₁-C₈alkyl) or SO₂—(C₁-C₈alkyl); or

R^{16a} and R^{17a} together with the N-atom to which they are attached form a 5- or 6-membered saturated or unsaturated ring which is uninterrupted or interrupted by O, S or N(C₁-C₈alkyl), and which 5- or 6-membered saturated or unsaturated ring is unsubstituted or substituted by one or more C₁-C₈alkyl, C₁-C₈alkoxy, C₁-C₈alkylsulfanyl, N(C₁-C₈alkyl)₂, NO₂, halogen, C₁-C₄haloalkyl, CN, phenyl or C₃-C₂₀cycloalkyl which is uninterrupted or interrupted by one or more O, S, CO or N(C₁-C₈alkyl);

R¹⁸ is hydrogen, C₁-C₂₀alkyl, C₁-C₄haloalkyl, C₂-C₂₀alkyl which is interrupted by one or more O, CO, C(O)O, or OC(CO), or is phenyl-C₁-C₄alkyl, C₃-C₈cycloalkyl which is uninterrupted or is interrupted by one or more O, CO, C₁-C₂₀alkyl, halogen, C₁-C₄haloalkyl, OR¹⁴, SR¹⁵, NR¹⁶R¹⁷ or

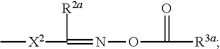

or

R¹⁸ is COR²¹; or

R¹⁸ is phenyl or naphthyl both of which are unsubstituted or substituted by one or more C₁-C₂₀alkyl, halogen, C₁-C₄haloalkyl, OR¹⁴, SR¹⁵, or NR¹⁶R¹⁷, COR¹³; or

R¹⁸ is C₁-C₆alkylene or C₂-C₆alkenylene linked to the phenyl or naphthyl ring of the carbazole moiety and forming a 5- or 6-membered saturated or unsaturated ring;

R¹⁹ is COOR¹⁴, CONR¹⁶R¹⁷, COR¹⁴; or

R¹⁹has one of the meanings given for R¹⁶ and R¹⁷;

R²⁰ is COOR¹⁴, CONR¹⁶R¹⁷, or COR¹⁴; or

R²⁰ has one of the meanings given for R¹⁴;

R²³, R²⁴, R²⁵ and R²⁶ independently of each other are hydrogen, C₁-C₂₀alkyl, COR¹³, COOR¹⁴, OR¹⁴, or NO₂;

k is an integer 1 to 10;

m is 1 or 2;

n is integer 1 to 20;

X¹ is O, S, SO or SO₂;

X² is O, CO, S or a direct bond;

M is C₁-C₂₀alkylene which is unsubstituted or substituted by one or more substituents selected from the group consisting of halogen, OR¹⁴, SR¹⁵, NR¹⁶R¹⁷, CN, COOR¹⁴, CONR¹⁶R¹⁷, C₃-C₈cycloalkyl which is uninterrupted or interrupted by one or more O, S, CO or NR¹⁶, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵, or NR¹⁶R¹⁷; or

M is C₂-C₂₀alkylene interrupted by one or more O, S, NR¹⁶, CO, SO or SO₂, which C₁-C₂₀alkylene or C₂-C₂₀alkylene is unsubstituted or substituted by one or more substituents selected from the group consisting of C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl and C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵, or NR¹⁶R¹⁷; or

M is C₂-C₁₂alkenylene or C₃-C₂₀cycloalkylene, which C₂-C₁₂alkenylene or C₃-C₂₀cycloalkylene is uninterrupted or interrupted by one or more O, S, CO, NR¹⁶ or COOR¹⁴; or

M is C₆-C₂₀arylene or C₃-C₂₀heteroarylene, which C₆-C₂₀arylene or C₃-C₂₀heteroarylene is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, COR^{3a} or SO₂R^{3a}; and

Y is a direct bond, O, S or NR¹⁸.

C₇-C₂₀alkyl is linear or branched and is, for example, C₁-C₁₈-, C₁-C₁₄-, C₁-C₁₂-, C₁-C₈-, C₁-C₆- or C₁-C₄alkyl. Examples are methyl, ethyl, propyl, isopropyl, n-butyl, sec-butyl, isobutyl, tert-butyl, pentyl, hexyl, heptyl, 2,4,4-trimethylpentyl, 2-ethylhexyl, octyl, nonyl, decyl, dodecyl, tetradecyl, pentadecyl, hexadecyl, octadecyl and icosyl.

C₁-C₂₀haloalkyl is C₁-C₂₀alkyl and C₁-C₄haloalkyl is C₁-C₄alkyl, which are mono- or poly-substituted by halogen up to the exchange of all H-atoms by halogen. Examples are chloromethyl, trichloromethyl, trifluoromethyl or 2-bromopropyl, especially trifluoromethyl or trichloro methyl.

C₂-C₂₀alkyl interrupted by one or more O, S, NR¹⁶, CO, SO or SO₂ is for example interrupted 1-9, 1-7 or once or twice by the defined radicals. In case the groups are interrupted by more than one O, said O-atoms are separated from one another by at least one methylene group, i.e. the O-atoms are non-consecutive. The alkyl groups in the interrupted alkyl are linear or branched. Examples are the following structural units —CH₂—O—CH₃, —CH₂CH₂—O—CH₂CH₃, —[CH₂CH₂O]_{y}—CH₃, with y=1-9, —(CH₂CH₂O)₇—CH₂CH₃, —CH₂—CH(CH₃)—O—CH₂—CH₂CH₃, or —CH₂—CH(CH₃)—O—CH₂CH₃, etc.

C₃-C₂₀cycloalkyl or C₃-C₈cycloalkyl is a mono- or polycyclic aliphatic ring, for example a mono-, bi- or tricyclic aliphatic ring, e.g. C₃-C₁₈-, C₃-C₁₂-, C₃-C₁₀cycloalkyl. Examples of monocyclic rings are cyclopropyl, cyclobutyl, cyclopentyl, cyclohexyl, or cycloheptyl, especially cyclopentyl and cyclohexyl. Examples of polycyclic rings are perhydroanthracyl, perhydrophenyathryl, perhydronaphthyl, perhydrofluorenyl, perhydrochrysenyl, perhydropicenyl, adamantyl, bicyclo[1.1.1]pentyl, bicyclo[4.2.2]decyl, bicyclo[2.2.2]octyl, bicyclo[3.3.2]decyl, bicyclo[4.3.2]undecyl, bicyclo[4.3.3]dodecyl, bicyclo[3.3.3]undecyl, bicyclo[4.3.1]decyl, bicyclo[4.2.1]nonyl, bicyclo[3.3.1]nonyl, bicyclo[3.2.1]octyl,

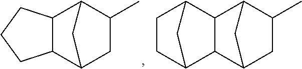

and the like.

Also “spiro”-cycloalkyl compounds are covered by the definition C₃-C₂₀cycloalkyl in the present context, e.g. spiro[5.2]octyl, spiro[5.4]decyl, spiro[5.5]undecyl. More examples of polycyclic cycloalkyl groups, which are subject of the respective definition in the compounds of the present invention are listed in EP 878738, page 11 and 12, wherein to the formulae (1)-(46) a bond to achieve the “yl” has to be added. The person skilled in the art is aware of this fact.

C₃-C₈cycloalkyl which is interrupted by one or more O, S, CO or NR¹⁶ refers to C₃-C₈cycloalkyl as defined above, wherein at least one C-atom is replaced by O, S, CO or NR¹⁶.

C₂-C₁₂alkenyl is mono or polyunsaturated, linear or branched and is for example C₂-C₈—, C₂-C₆- or C₂-C₄alkenyl. Examples are allyl, methallyl, vinyl, 1,1-dimethylallyl, 1-butenyl, 3-butenyl, 2-butenyl, 1,3-pentadienyl, 5-hexenyl or 7-octenyl, especially allyl or vinyl.

C₁-C₂₀alkoxy is linear or branched and is for example C₁-C₁₈-, C₁-C₁₆-, C₁-C₁₂-, C₁-C₈, C₁-C₆- or C₁-C₄-alkoxy. Examples are methoxy, ethoxy, propoxy, isopropoxy, n-butyloxy, sec-butyloxy, iso-butyloxy, tert-butyloxy, pentyloxy, hexyloxy, heptyloxy, 2,4,4-trimethylpetyloxy, 2-ethylhexyloxy, octyloxy, nonyloxy, decyloxy, dodecyloxy, hexadecyloxy, octadecyloxy or icosyloxy, in particular methoxy, ethoxy, propoxy, isopropoxy, n-butyloxy, sec-butyloxy, iso-butyloxy, tert-butyloxy, especially methoxy.

C₂-C₂₀alkoxy, which is interrupted by one or more O, S, NR¹⁶, CO, SO or SO₂ is for example interrupted 1-9, 1-7, 1-4 or once or twice by the defined radicals. In case the groups are interrupted by more than one O, said O-atoms are separated from one another by at least one methylene group, i.e. the O-atoms are non-consecutive. Examples are the following structural units —O—CH₂—O—CH₃, —O—CH₂CH₂—O—CH₂CH₃, —O—[CH₂CH₂O]ᵥCH₃, with v=1-4, —O—(CH₂CH₂O)₄CH₂CH₃, —O—CH₂—CH(CH₃)—O—CH₂—CH₂CH₃, or —O—CH₂—CH(CH₃)—O—CH₂CH₃, etc.

C₁-C₈alkylsulfanyl (═C₁-C₈alkylthio) is C₁-C₈alkyl (as defined above), which at the “yl” moiety bears one-S-atom. C₁-C₈alkylsulfanyl is linear or branched, for example, methylsulfanyl, ethylsulfanyl, propylsulfanyl, isopropylsulfanyl, n-butylsulfanyl, sec-butylsulfanyl, isobutylsulfanyl, tert-butylsulfanyl, etc.

C₁-C₂₀alkanoyl (═C₁-C₂₀alkylcarbonyl) is linear or branched and is, for example, C₁-C₁₈-, C₁-C₁₄-, C₁-C₁₂-, C₁-C₈-, C₁-C₆- or C₁-C₄alkanoyl or C₄-C₁₂- or C₄-C₈alkanoyl. Examples are formyl, acetyl, propionyl, butanoyl, isobutanoyl, pentanoyl, hexanoyl, heptanoyl, octanoyl, nonanoyl, decanoyl, dodecanoyl, tetradecanoyl, pentadecanoyl, hexadecanoyl, octadecanoyl, icosanoyl, preferably acetyl.

C₃-C₁₂alkenoyl is mono or polyunsaturated. Examples are propenoyl, 2-methyl-propenoyl, butenoyl, pentenoyl, 1,3-pentadienoyl, 5-hexenoyl, etc.

Halogen is fluorine, chlorine, bromine and iodine, especially fluorine, chlorine and bromine, preferably fluorine and chlorine.

C₁-C₂₀alkylphenyl corresponds to phenyl that is substituted once or more times by alkyl at the phenyl ring and is for example C₁-C₁₂alkyl-, C₁-C₈alkyl- or C₁-C₄alkylphenyl, wherein the number of the alkyl corresponds to the total number of all C-atoms in all alkyl-substituents at the phenyl ring. Examples aretolyl, xylyl, mesityl, ethylphenyl, diethylphenyl, in particular tolyl and mesityl.

C₁-C₈alkoxyphenyl corresponds to phenyl that is substituted once or more times by alkoxyl at the phenyl ring and is for example C₁-C₄alkoxyphenyl, wherein the number of the alkoxy corresponds to the total number of all C-atoms in all alkoxy-substituents at the phenyl ring.

Examples are methoxyphenyl, dimethoxyphenyl, trimethoxyphenyl, ethoxyphenyl, diethoxyphenyl, ect.

C₆-C₂₀aryl is for example phenyl, naphthyl, anthryl or phenanthryl, in particular phenyl or naphthyl, preferably phenyl.

Naphthyl corresponds to 1-naphthyl and 2-naphthyl.

C₆-C₂₀aroyl corresponds to C₆-C₂₀aryl-CO—, wherein C₆-C₂₀aryl is defined as above. Examples are benzoyl, naphthoyl etc.

C₆-C₂₀aryloxy corresponds to C₆-C₂₀aryl-O—, wherein C₆-C₂₀aryl is defined as above. Examples are phenyoxy, naphthyloxy etc.

Phenyl-C₁-C₃alkyloxy corresponds to C₁-C₃alkyloxy (═C₁-C₃alkoxy) which is substituted by phenyl.

C₃-C₂₀heteroaryl is meant to comprise either a one ring or a multiple ring system, e.g. a fused ring-system. Examples are thienyl, benzo[b]thienyl, naphtho[2,3-b]thienyl, thianthrenyl, furyl, dibenzofuryl, benzofuryl, chromenyl, xanthenyl, thioxanthyl, phenoxathiinyl, pyrrolyl, imidazolyl, pyrazolyl, pyrazinyl, pyrimidinyl, pyridazinyl, indolizinyl, isoindolyl, indolyl, indazolyl, purinyl, quinolizinyl, isoquinolyl, quinolyl, phthalazinyl, naphthyridinyl, quinoxalinyl, quinazolinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, 7-phenanthryl, anthraquinone-2-yl (=9,10-dioxo-9,10-dihydroanthracen-2-yl), 3-benzo[b]thienyl, 5-benzo[b]thienyl, 2-benzo[b]thienyl, 4-dibenzofuryl, 4,7-dibenzofuryl, 4-methyl-7-dibenzofuryl, 2-xanthenyl, 8-methyl-2-xanthenyl, 3-xanthenyl, 2-phenoxyathiinyl, 2,7-phenoxathiinyl, 2-pyrrolyl, 3-pyrrolyl, 5-methyl-3-pyrrolyl, 2-imidazolyl, 4-imidazolyl, 5-imidazolyl, 2-methyl-4-imidazolyl, 2-ethyl-4-imidazolyl, 2-ethyl-5-imidazolyl, 1H-tetrazol-5-yl, 3-pyrazolyl, 1-methyl-3-pyrazolyl, 1-propyl-4-pyrazolyl, 2-pyrazinyl, 5,6-dimethyl-2-pyrazinyl, 2-indolizinyl, 2-methyl-3-isoindolyl, 2-methyl-1-isoindolyl, 1-methyl-2-indolyl, 1-methyl-3-indolyl, 1,5-dimethyl-2-indolyl, 1-methyl-3-indazolyl, 2,7-dimethyl-8-purinyl, 2-methoxy-7-methyl-8-purinyl, 2-quinolizinyl, 3-isoquinolyl, 6-isoquinolyl, 7-isoquinolyl, 3-methoxy-6-isoquinolyl, 2-quinolyl, 6-quinolyl, 7-quinolyl, 2-methoxy-3-quinolyl, 2-methoxy-6-quinolyl, 6-phthalazinyl, 7-phthalazinyl, 1-methoxy-6-phthalazinyl, 1,4-dimethoxy-6-phthalazinyl, 1,8-naphthyridin-2-yl, 2-quinoxalinyl, 6-quinoxalinyl, 2,3-dimethyl-6-quinoxalinyl, 2,3-dimethoxy-6-quinoxalinyl, 2-quinazolinyl, 7-quinazolinyl, 2-dimethylamino-6-quinazolinyl, 3-cinnolinyl, 6-cinnolinyl, 7-cinnolinyl, 3-methoxy-7-cinnolinyl, 2-pteridinyl, 6-pteridinyl, 7-pteridinyl, 6,7-dimethoxy-2-pteridinyl, 2-carbazolyl, 3-carbazolyl, 9-methyl-2-carbazolyl, 9-methyl-3-carbazolyl, β-carbolin-3-yl, 1-methyl-β-carbolin-3-yl, 1-methyl-β-carbolin-6-yl, 3-phenanthridinyl, 2-acridinyl, 3-acridinyl, 2-perimidinyl, 1-methyl-5-perimidinyl, 5-phenanthrolinyl, 6-phenanthrolinyl, 1-phenazinyl, 2-phenazinyl, 3-isothiazolyl, 4-isothiazolyl, 5-isothiazolyl, 2-phenothiazinyl, 3-phenothiazinyl, 10-methyl-3-phenothiazinyl, 3-isoxazolyl, 4-isoxazolyl, 5-isoxazolyl, 4-methyl-3-furazanyl, 2-phenoxazinyl, 10-methyl-2-phenoxazinyl, etc.

C₃-C₂₀heteroaryl in particular is thienyl, benzo[b]thienyl, furyl, benzofuryl, thianthrenyl, thioxanthyl, 1-methyl-2-indolyl or 1-methyl-3-indolyl.

C₃-C₂₀heteroarylcarbonyl corresponds to C₃-C₂₀heteroaryl-CO—, wherein C₃-C₂₀heteroaryl is defined as above.

C₃-C₂₀heteroaryloxy corresponds to C₃-C₂₀heteroaryl-O—, wherein C₃-C₂₀heteroaryl is defined as above.

C₁-C₂₀alkylene is linear or branched and is for example, C₁-C₁₈-, C₁-C₁₄-, C₁-C₁₂-, C₁-C₈-, C₁-C₆- or C₁-C₄alkylene. Examples are methylene, ethylene, propylene, 1-methylethylene 1,1-dimethylethylene, butylene, 1-methylpropylene, 2-methyl-propylene, pentylene, hexylene, heptylene, octylene, nonylene, decylene, dodecylene, tetradecylene, hexadecylene or octadecylene.

C₂-C₂₀alkylene interrupted by one or more O, S, NR₅, CO, SO or SO₂ is, for example, interrupted 1-9 times, for example 1-7 times or once or twice by the defined substituents and the C₂-C₂₀alkylene is linear or branched. This produces structural units such as, for example, —CH₂—O—CH₂—, —CH₂—S—CH₂—, —CH₂—N(CH₃)—CH₂—, —CH₂CH₂—O—CH₂CH₂—, —[CH₂CH₂O]_{y}—, —[CH₂CH₂O]_{y}—CH₂—, where y=1-9, —(CH₂CH₂O)₇CH₂CH₂—, —CH₂—CH(CH₃)—O—CH₂—CH(CH₃)— or —CH₂—CH(CH₃)—O—CH₂—CH₂CH₂—. Interrupting O-atoms are non-successive.

C₂-C₁₂alkenylene is mono- or polyunsaturated, linear or branched and is, for example, C₁-C₈-, C₁-C₆- or C₁-C₄alkenylene. Examples are ethenylene, 1-propenylene, 1-butenylene, 3-butenylene, 2-butenylene, 1,3-pentadienylene, 5-hexenylene, 7-octenylene, etc.

C₃-C₂₀cycloalkylene consists of a two valent mono- or polycyclic aliphatic ring, for example a mono-, bi- or tricyclic aliphatic ring, e.g. C₃-C₁₈-, C₃-C₁₂-, C₃-C₁₀cycloalkylene. Examples are cyclopropylene, cyclopentylene, cyclohexylene, cyclooctylene, cyclododecylene,

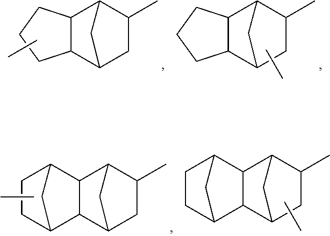

especially cyclopentylene and cyclohexylene, preferably cyclohexylene.

C₆-C₂₀arylene is for example phenylene, biphenylene, o-, m- and p-terphenylene, triphenylphenylene, naphthylene, binaphthylene, anthracenylene, phenanthrylene or pyrenylene, in particular phenylene or naphthylene, especially phenylene.

C₃-C₂₀heteroarylene refers to C₃-C₂₀heteroaryl as defined above however instead of being one-valent C₃-C₂₀heteroarylene groups are two valent.

If R¹⁶ and R¹⁷ together with the N-atom to which they are attached form a 5- or 6-membered saturated or unsaturated ring which is uninterrupted or interrupted by O, S or NR₅ₐ, saturated or unsaturated rings are formed, for example aziridine, pyrrole, thiazole, pyrrolidine, oxazole, pyridine, 1,3-diazine, 1,2-diazine, piperidine or morpholine. Preferably, if R¹⁶ and R¹⁷ together with the N-atom to which they are attached form a 5- or 6-membered saturated or unsaturated ring which optionally is interrupted by O, S or NR^{16a}, 5- or 6-membered saturated rings which are not interrupted or which are interrupted by O or NR^{16a}, in particular by O, are formed.

If R^{16a} and R^{17a} together with the N-atom to which they are attached form a 5- or 6-membered saturated or unsaturated ring which is uninterrupted or interrupted by O, S or N(C₁-C₈alkyl), saturated or unsaturated rings are formed as described above for R₅ and R₆ forming a ring with the N-atom.

Substituted aryl radicals phenyl, naphthyl, C₆-C₂₀aryl, C₅-C₂₀heteroaryl, C₆-C₂₀arylene or C₃-C₂₀heteroarylene etc. are substituted 1 to 7, 1 to 6 or 1 to 4 times respectively, in particular one, two or three times. It is evident that a defined aryl or heteroaryl radical cannot have more substituents than free “CH” or “NH” positions are at the defined ring.

Substituents on the phenyl ring are preferably in positions 4 or in 3,4-, 3,4,5-, 2,6-, 2,4- or 2,4,6-configuration on the phenyl ring.

Interrupted radicals which are interrupted once or more times are for example interrupted 1-19, 1-15, 1-12, 1-9, 1-7, 1-5, 1-4, 1-3 or once or twice (it is evident, that the number of interrupting atoms depends on the number of C-atoms to be interrupted).

Substituted radicals, which are substituted once or more times have for example 1-7, 1-5, 1-4, 1-3 or one or two identical or different substituents.

A radical substituted by one or more defined substituents is meant to have either one substituent or more substituents of identical or different definitions as given

The terms “and/or” or “or/and” in the present context are meant to express that not only one of the defined alternatives (substituents) may be present, but also several of the defined alternatives (substituents) together, namely mixtures of different alternatives (substituents).

The term “at least” is meant to define one or more than one, for example one or two or three, preferably one or two.

The term “optionally substituted” means, that the radical to which it refers is either unsubstituted or substituted.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The term “(meth)acrylate” in the context of the present application is meant to refer to the acrylate as well as to the corresponding methacrylate.

The preferences indicated above for the compounds according to the present invention in the context of this invention are intended to refer to all categories of the claims, that is to the compositions, use, process claims as well.

It is to be understood that this invention is not limited to particular compounds, configurations, method steps, substrates, and materials disclosed herein as such compounds, configurations, method steps, substrates, and materials may vary somewhat. It is also to be understood that the terminology employed herein is used for the purpose of describing particular embodiments only and is not intended to be limiting since the scope of the present invention is limited only by the appended claims and equivalents thereof.

It must be noted that, as used in this specification and the appended claims, the singular forms “a”, “an” and “the” include plural referents unless the context clearly dictates otherwise.

If nothing else is defined, any terms and scientific terminology used herein are intended to have the meanings commonly understood by those of skill in the art to which this invention pertains.

General Procedure of Preparing the Novel Oxime Esters

The novel oxime ester compounds of the present invention can be synthesized, for example, by the following method, but are not limited to this.

Oxime esters formula I are prepared by methods described in the literature, for example by reaction of the corresponding oximes with an acyl halide, in particular a chloride, or an anhydride in an inert solvent such as for example t-butyl methyl ether (TBME), tetrahydrofurane (THF), dimethoxyethane (DME), dimethylacetamide (DMA), ethyl acetate or dimethylformamide (DMF) in the presence of a base or a mixture of bases, for example triethylamine or pyridine, or in a basic solvent such as pyridine. As example in the following the preparation of compounds of the formula I is described:

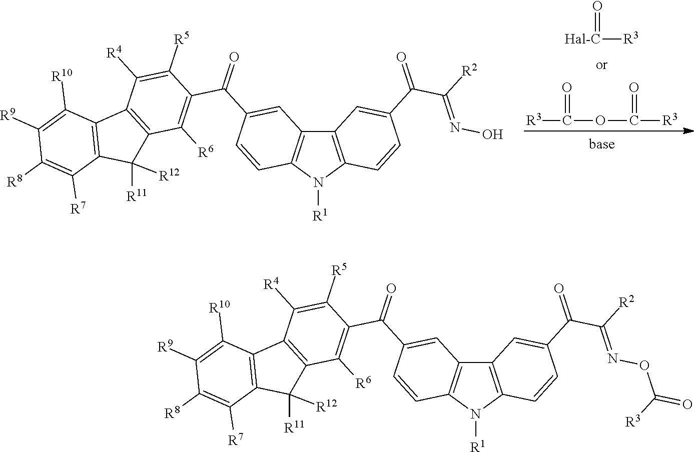

R³ have the meanings as given above, and R³ is preferably methyl. Hal means a halogen atom, in particular Cl. Such reactions are well known to those skilled in the art such as WO2012045736, and are generally carried out at temperatures of −15 to +50° C., preferably 0 to 25° C.

Every oxime ester group can exist in two configurations, (Z) or (E). It is possible to separate the isomers by conventional methods, but it is also possible to use the isomeric mixture as such as photoinitiator species. Therefore, the invention also relates to mixtures of configurational isomers of compounds of the formula I.

The α-ketoximes required as starting materials can be obtained by a variety of methods described in standard chemistry textbooks (for instance in J. March, Advanced Organic Chemistry, 4^{th} Edition, Wiley Interscience, 1992), or in specialized monographs, for example, S. R. Sandler & W. Karo, Organic functional group preparations, Vol. 3, Academic Press. One of the most convenient methods is, for example, the nitrosation of “active” methylene groups with nitrous acid or an alkyl nitrite. Both alkaline conditions, as described for example in Organic Synthesis coll. Vol. VI (J. Wiley & Sons, New York, 1988), pp 199 and 840, and acidic conditions, as described, for example, in Organic Synthesis coll. Vol. V, pp 32 and 373, coll. Vol. III, pp 191 and 513, coll. Vol. II, pp 202, 204 and 363, are suitable for the preparation of the oximes used as starting materials in the invention. Nitrous acid is usually generated from sodium nitrite. The alkyl nitrite can be for example methyl nitrite, ethyl nitrite, isopropyl nitrite, butyl nitrite, amyl nitrite, or isoamyl nitrite.

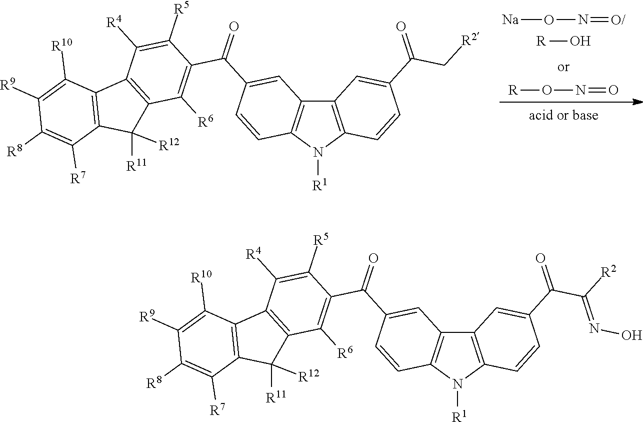

The corresponding ketone intermediates are for example prepared by the methods described in the literature, for example, in standard chemistry textbooks (for instance in J. March. Advanced Organic Chemistry, 4^{th} Edition, Wiley Interscience, 1992). In addition, successive Friedel-Clafts reaction is effective for synthesis of the intermediates. Such reactions are well known to those skilled in the art.

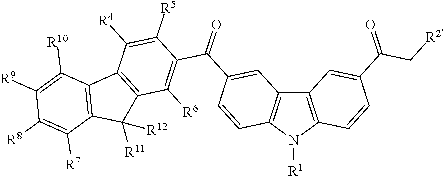

The corresponding ketone intermediates can be synthesized, for example, by the following method, but are not limited to this. The plausible synthetic schemes are described as follows. Acylation of [A], and coupling reaction of [B] or [D] with [C] or [F] give the corresponding ketone intermediate. Additionally, coupling of [D] with fluorene also gives the corresponding ketone intermediate via alkylation of [E] (Scheme 1).

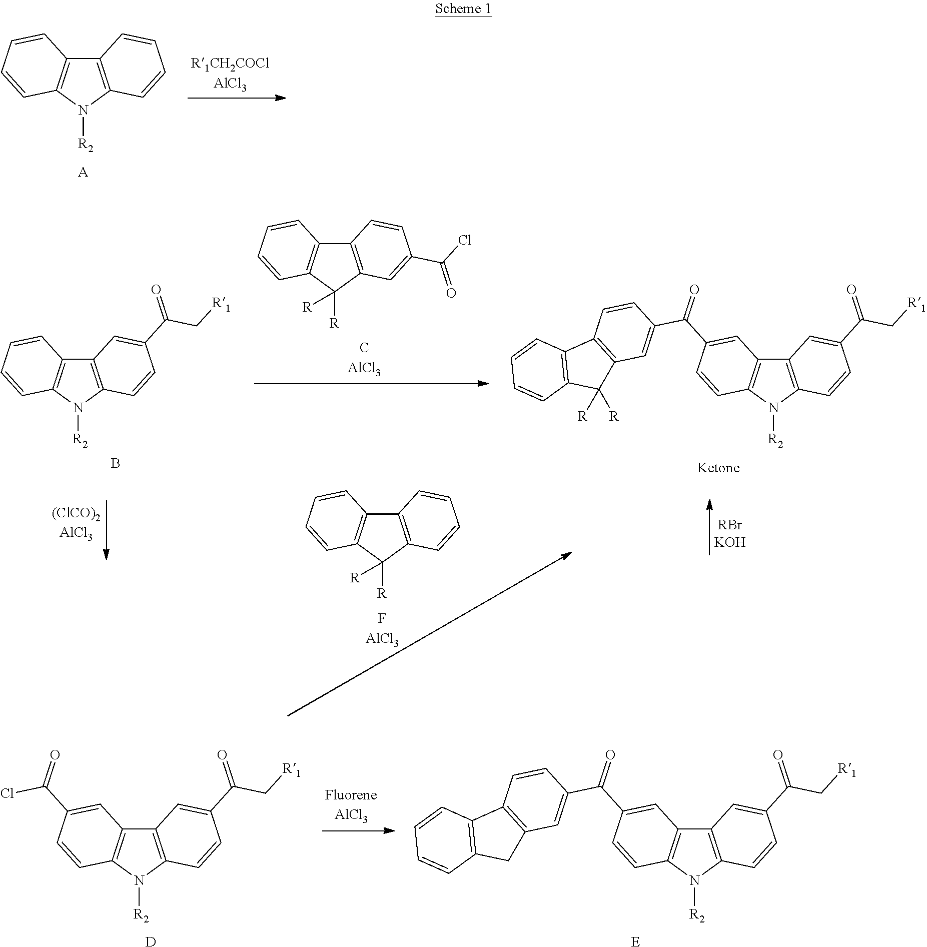

As another plausible synthetic scheme is described as follows. Coupling reaction of [G] derived from fluorene with [B] gives intermediate [E], Then, alkylation of [E] gives the corresponding ketone intermediate. Additionally, [G] can also react with [A] to form [H], which is converted to [E] by acylation. Alkylation of [H] to form [I], and next acylation reaction give the corresponding ketone intermediate. [I] is also synthesized by coupling reaction of [A] with [C] (Scheme 2).

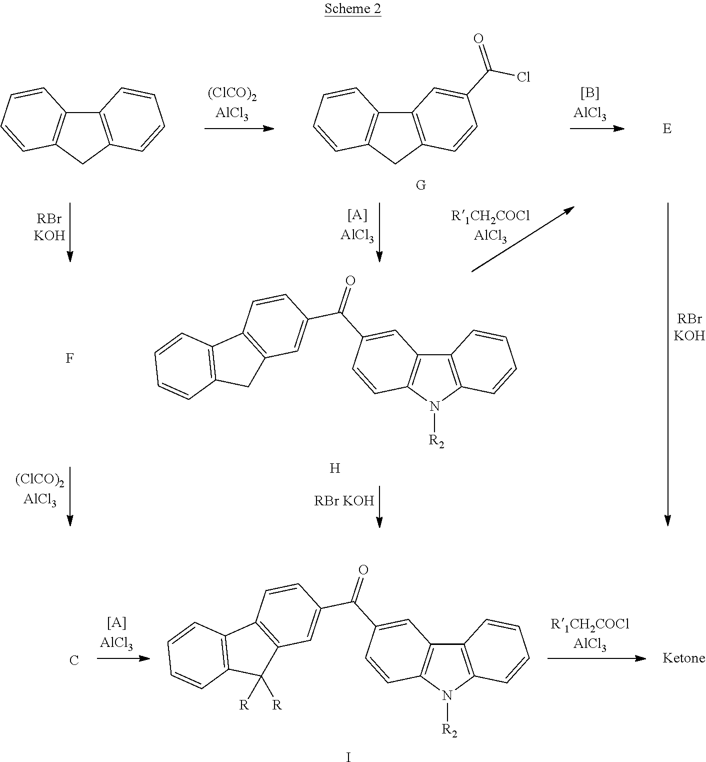

Subject of the invention also is a process for the preparation of a compound of the formula I as defined above by reacting the corresponding oxime compound of the formula (IIA) as defined above with an acyl halide of the formula I′ or an anhydride of the formula I″

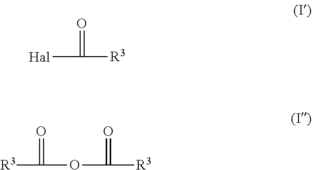

wherein Hal is a halogen, in particular Cl, and R³ is as defined above, in the presence of a base or a mixture of bases;

and the compounds of the Formula IA

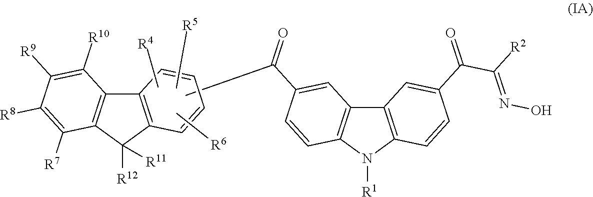

wherein R¹, R² and R⁴ to R¹² are defined above.

Interesting compounds of the formula I as shown above include compounds of formula II:

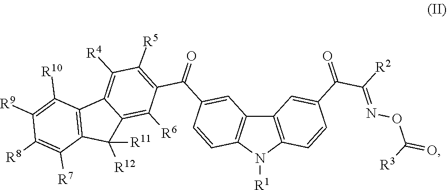

R¹ is C₁-C₂₀alkyl which is unsubstituted or substituted by one or more halogen, OR¹⁴, SR¹⁵, COOR¹⁴, NR¹⁶R¹⁷, CONR¹⁶R¹⁷, PO(OCₖH₂ₖ₊₁)₂; or

R¹ is C₂-C₂₀alkyl which is interrupted by one or more O, CO, S, C(O)O, OC(O), phenylene, naphthylene or NR¹⁸, wherein the interrupted C₁-C₂₀alkyl is unsubstituted or substituted by one or more halogen, OR¹⁴, SR¹⁵, COOR¹⁴ NR¹⁶R¹⁷, CONR¹⁶R¹⁷; or

R¹ is C₆-C₂₀aryl or C₃-C₂₀heteroaryl each of which is unsubstituted or substituted by one or more phenyl, halogen, C₁-C₄haloalkyl, CN, NO₂, OR¹⁴, SR¹⁵, NR¹⁶R¹⁷,

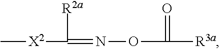

or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷;

R² is hydrogen, C₁-C₂₀alkyl which is unsubstituted or substituted by one or more substituents halogen, OR¹⁴, SR¹⁵, NR¹⁶R¹⁷, COOR¹⁴,

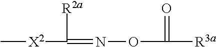

or C₃-C₈cycloalkyl which is uninterrupted or interrupted by one or more O; or

R² is C₂-C₂₀alkyl interrupted by one or more O, which interrupted C₂-C₂₀alkyl is unsubstituted or substituted by one or more C₃-C₈cycloalkyl, OH, O(CO)R^{3a} or COOR¹⁴; or

R² is C₂-C₁₂alkenyl, C₃-C₂₀cycloalkyl or C₆-C₂₀aryl, which C₆-C₂₀aryl or C₃-C₂₀heteroaryl is unsubstituted or substituted by one or more halogen, NO₂, OR¹⁴, SR¹⁵, COOR¹⁴, COR^{3a} or

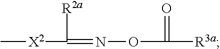

R^{2a} is hydrogen, COR^{3a}, COOR^{14a}, C₁-C₂₀alkyl which is unsubstituted or substituted by one or more halogen, OR^{14a}, SR^{15a} or C₃-C₈cycloalkyl which is uninterrupted or interrupted by one or more O;

R³ is C₁-C₂₀alkyl which is unsubstituted or substituted by one or more halogen, OR¹⁴, SR¹⁵, NR¹⁶R¹⁷, COOR¹⁴, C₃-C₈cycloalkyl or C₃-C₈cycloalkyl which is interrupted by one or more O; or

R³ is C₂-C₂₀alkyl interrupted by one or more O, which interrupted C₂-C₂₀alkyl is unsubstituted or substituted by one or more OH, O(CO)R^{3a} or COOR¹⁴; or

R³ is C₂-C₁₂alkenyl or C₃-C₂₀cycloalkyl; or

R³ is C₆-C₂₀aryl or C₃-C₂₀heteroaryl, each of which is unsubstituted or substituted by one or more halogen or C₁-C₂₀alkyl; or

R³ is C₁-C₂₀alkoxy, C₆-C₂₀aryloxy or C₃-C₂₀heteroaryloxy;

R^{3a} is hydrogen, C₁-C₂₀alkyl, C₂-C₂₀alkyl interrupted by one or more O, C₂-C₁₂alkenyl, C₃-C₂₀cycloalkyl, C₆-C₂₀aryl or C₃-C₂₀heteroaryl, which C₆-C₂₀aryl or C₃-C₂₀heteroaryl is unsubstituted or substituted by one or more halogen, C₁-C₂₀alkyl, OR^{14a}, SR^{15a}, NR^{16a}R^{17a}, COOR^{14a}, (CO)—(C₁-C₈alkyl) or benzoyl; or

R^{3a} is C₆-C₂₀aryloxy or C₃-C₂₀heteroaryloxy, which C₆-C₂₀aryloxy or C₃-C₂₀heteroaryloxy is unsubstituted or substituted by benzoyl, or

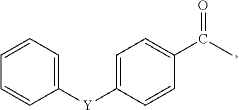

which benzoyl or

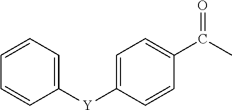

unsubstituted or substituted by

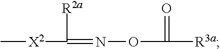

R⁴ to R¹², and Y are defined as above;

R¹⁴ is hydrogen, COR^{3a}, C₁-C₂₀alkyl which is unsubstituted or substituted by one or more C₃-C₈cycloalkyl which is uninterrupted or interrupted by one or more O; or

R¹⁴ is C₂-C₂₀alkyl interrupted by one or more O, which interrupted C₂-C₂₀alkyl is unsubstituted or substituted by one or more C₃-C₈cycloalkyl, OH or COOR^{14a}; or

R¹⁴ is C₂-C₁₂alkenyl, C₃-C₂₀cycloalkyl or C₆-C₂₀aryl, which C₆-C₂₀aryl is unsubstituted or substituted by one or more halogen or C₁-C₂₀alkyl; or

R^{14a} is C₁-C₂₀alkyl; R¹⁵ is hydrogen, C₁-C₁₀alkyl which is unsubstituted or substituted by one or more OR^{14a}, COOR^{14a}, C₃-C₈cycloalkyl or C₆-C₂₀aryl; or

R¹⁵ is C₂-C₂₀alkyl interrupted by one or more O, which interrupted C₂-C₂₀alkyl is unsubstituted or substituted by one or more OH or COOR^{14a}; or

R¹⁵ is C₂-C₁₂alkenyl or C₃-C₂₀cycloalkyl; or

R¹⁵ is C₆-C₂₀aryl which is unsubstituted or substituted by one or more halogen or C₂-C₂₀alkyl which is uninterrupted or interrupted by one or more O;

R^{15a} is C₁-C₂₀alkyl;

R¹⁶ and R¹⁷ independently of each other are hydrogen, C₁-C₂₀alkyl which is unsubstituted or substituted by one or more halogen, OR^{14a}, SR^{15a}, COOR^{14a}, C₃-C₈cycloalkyl or C₆-C₂₀aryl; or

R¹⁶ and R¹⁷ independently of each other are C₂-C₂₀alkyl interrupted by one or more O, which interrupted C₂-C₂₀alkyl is unsubstituted or substituted by OH or SH; or

R¹⁶ and R¹⁷ independently of each other are C₂-C₁₂alkenyl, C₃-C₂₀cycloalkyl or C₆-C₂₀aryl; or

R¹⁶ and R¹⁷ together with the N-atom to which they are attached form a 5- or 6-membered saturated or unsaturated ring which is uninterrupted or interrupted by O, S or NR^{16a}; R^{16a} and R^{17a} independently of each other are hydrogen or C₁-C₂₀alkyl; and X² is CO or a direct bond.

Preferred compounds of the formula I according to the present invention include compounds of formula III:

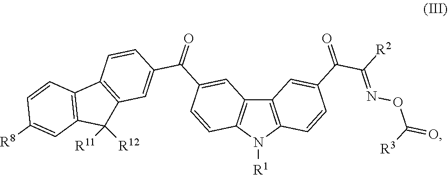

wherein

R¹ is C₁-C₂₀alkyl which is unsubstituted or substituted by NR¹⁶R¹⁷; or

R¹ is C₂-C₂₀alkyl which is interrupted by one or more C(O)O, OC(O) or phenylene, wherein the interrupted C₁-C₂₀alkyl is unsubstituted or substituted by NR¹⁶R¹⁷; or

R¹ is phenyl or thienyl each of which is unsubstituted or substituted by

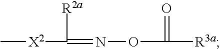

R² is C₁-C₂₀alkyl which is unsubstituted or substituted by halogen, SR¹⁵, NR¹⁶R¹⁷, COOR¹⁴ or C₃-C₈cycloalkyl; or

R² is C₆-C₂₀aryl which is unsubstituted or substituted by C₁-C₂₀alkyl;

R^{2a} is C₁-C₂₀alkyl or C₆-C₂₀aryl;

R^{2a} is C₆-C₂₀aryl which is unsubstituted or substituted by C₁-C₂₀alkyl; or

R³ is C₁-C₂₀alkyl, C₆-C₂₀aryl or C₁-C₂₀alkoxy;

R^{3a} is C₁-C₂₀alkyl or C₆-C₂₀aryl;

R⁸ is H, NO₂, or COR¹³, wherein

R¹¹ and R¹² independently of each other are hydrogen or C₁-C₂₀alkyl,

R¹³ is phenyl, naphthyl or thienyl which is unsubstituted and substituted by phenyl, C₁-C₂₀alkyl, OR¹⁴, SR¹⁵, or NR¹⁶R¹⁷, wherein

R¹⁴ is C₁-C₂₀alkyl or R¹⁴ together with one of the carbon atoms of R² forms a 5- or 6-membered, preferably 6-membered, saturated ring;

R¹⁵ is C₆-C₂₀aryl; and

R¹⁶ and R¹⁷ independently of each other are O(CO)R^{3a}, COR^{3a} or C₁-C₂₀alkyl.

For example, illustrative compounds of formula III include e.g. compounds of formula III in which R¹ to R³, R¹¹ and R¹² are as defined above; R⁸ is H, NO₂, or COR¹³, wherein R¹³ is phenyl, naphthyl or thienyl which is unsubstituted and substituted by phenyl, C₁-C₂₀alkyl, OR¹⁴, SR¹⁵, or NR¹⁶R¹⁷, wherein R¹⁴ to R¹⁷ are as defined above.

It is preferred according to the invention that the compounds of the formula I are compounds of formula IV:

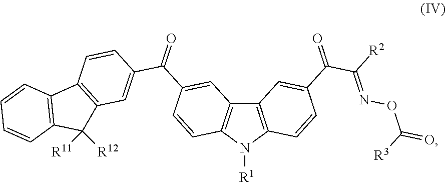

wherein R¹ to R³, R¹¹ and R¹² are defined as above.

The compounds of the formula I are suitable as radical photoinitiators.

Accordingly, subject of the invention is the use of a compound of the formula I as defined above for the photopolymerization of a composition comprising at least one ethylenically unsaturated photopolymerizable compound.

Another subject of the present invention therefore is a photopolymerizable composition comprising

(a) at least one ethylenically unsaturated photopolymerizable compound and

(b) as photoinitiator, at least one compound of the formula I as defined above.

The composition may comprise additionally to the photoinitiator (b) at least one further photoinitiator (c) or other additives (d), or both, at least one further photoinitiator (c) and other additives (d).

Other additives (d) for example are one or more components selected from thermal radical initiators, pigments, fillers, dispersants and sensitizers.

Said additives are described in more detail below.

Interesting is a photopolymerizable composition as described above, comprising 0.05 to 25% by weight of the photoinitiator (b), or the photoinitiators (b) and (c), based on the composition.

The unsaturated compounds (a) may include one or more olefinic double bonds. They may be of low (monomeric) or high (oligomeric) molecular mass.

Component (a) for example comprises a resin obtained by the reaction of a saturated or unsaturated polybasic acid anhydride with a product of the reaction of an epoxy resin and an unsaturated monocarboxylic acid.

Subject of the invention accordingly also is a photopolymerizable composition as described above, wherein the component (a) is a resin obtained by the reaction of a saturated or unsaturated polybasic acid anhydride with a product of the reaction of an epoxy resin and an unsaturated monocarboxylic acid.

Component (a) for example comprises an alkaline developable resin.

The polymerizable composition according to the invention preferably comprises the component (a) in an amount of from 2 to 98% by weight, more preferably from 5 to 90% by weight, in particular from 10 to 80% by weight, based on the whole solid contents of the polymerizable composition (i.e. the amount of all components without the solvent(s)).

Preferably, the alkaline developable resin has free carboxylic groups. The acid number is preferably from 50 to 600 mg KOH/g, more preferably 100 to 300 mg KOH/g. The acid numbers stated here are the acid number according to DIN EN 12634.

Examples of alkali developable resins are acrylic polymers having carboxylic acid function as a pendant group, such as copolymers obtained by copolymerizing an ethylenic unsaturated carboxylic acid such as (meth)acrylic acid, 2-carboxyethyl (meth)acrylic acid, 2-carboxypropyl (meth)acrylic acid, itaconic acid, citraconic acid, mesaconic acid, fumaric acid, crotonic acid, maleic acid, maleic anhydride, fumaric anhydride, half-ester of maleic acid, cinnamic acid, mono[2-(meth)acryloyloxyethyl] succinate, mono[2-(meth)acryloyloxyethyl] adipate, mono[2-(meth)acryloyloxyethyl] phthalate, mono[2-(meth)acryloyloxyethyl] hexahydrophthalate, mono[2-(meth)acryloyloxyethyl] maleate, mono[2-(meth)acryloyloxypropyl] succinate, mono[2-(meth)acryloyloxypropyl] adipate, mono[2-(meth)acryloyloxypropyl] phthalate, mono[2-(meth)acryloyloxypropyl] hexahydrophthalate, mono[2-(meth)acryloyloxypropyl] maleate, mono[2-(meth)acryloyloxybutyl] succinate, mono[2-(meth)acryloyloxybutyl] adipate, mono[2-(meth)acryloyloxybutyl] phthalate, mono[2-(meth)acryloyloxybutyl] hexahydrophthalate, mono[2-(meth)acryloyloxybutyl] maleate, 3-(alkylcarbamoyl)acrylic acid, α-chloroacrylic acid, monoesterified maleic acid, and ω-carboxypolycaprolactone mono(meth)acrylate, with one or more monomers selected from esters of (meth)acrylic acid, such as methyl (meth)acrylate, ethyl (meth)acrylate, propyl (meth)acrylate, butyl (meth)acrylate, benzyl (meth)acrylate, 2-ethylhexyl (meth)acrylate, hydroxyethyl (meth)acrylate, hydroxypropyl (meth)acrylate, tetrahydrofurfuryl (meth)acrylate, hydroxybutyl (meth)acrylate, glycerol mono(meth)acrylate, dihydroxypropyl (meth)acrylate, allyl (meth)acrylate, cyclohexyl (meth)acrylate, phenyl (meth)acrylate, methoxyphenyl (meth)acrylate, methoxyethyl (meth)acrylate, phenoxyethyl (meth)acrylate, methoxydiethyleneglycol (meth)acrylate, methoxytriethyleneglycol (meth)acrylate, methoxypropyl (meth)acrylate, methoxydipropyleneglycol (meth)acrylate, (3-trimethoxysilyl)propyl (meth)acrylate, (meth)acrylic acid trimethylsilyl ester, isobornyl meth(acrylate), dicyclopentadienyl (meth)acrylate, 2-hydroxy-3-phenoxypropyl (meth)acrylate, tricyclo[5.2.1.0^{2,6}]decan-8-yl (meth)acrylate, aminoethyl (meth)acrylate, N, N-dimethylaminoethyl (meth)acrylate, aminopropyl (meth)acrylate, N, N-dimethylaminopropyl (meth)acrylate, glycidyl (meth)acrylate, 2-methylglycidyl (meth)acrylate, 3,4-epoxybutyl (meth)acrylate, 6,7-epoxyheptyl (meth)acrylate; vinyl aromatic compounds, such as styrene, α-methylstyrene, vinyltoluene, p-chlorostyrene, polychlorostyrene, fluorostyrene, bromostyrene, ethoxymethyl styrene, methoxystyrene, 4-methoxy-3-methystyrene, dimethoxystyrene, vinylbenzyl methyl ether, vinylbenzyl glycidyl ether, indene, 1-methylindene; 1-ethenyl-4-silyl-benzen, 1-ethenyl-4-trimethylsilyl-benzene, t-buthyl dimethylsilyl p-vinyl phenyl ester; amide type unsaturated compounds, such as (meth)acrylamide, diacetone acrylamide, N-methylolacrylamide, N-butoxymethacrylamide, N,N-dimethyl (meth)acrylamide, N,N-diethyl (meth)acrylamide, N,N-dibutyl (meth)acrylamide, N,N-diethylhexyl (meth)acrylamide, N,N-dicyclohexyl (meth)acrylamide, N,N-diphenyl (meth)acrylamide, N-methyl-N-phenyl (meth)acrylamide, N-hydroxyethyl-N-methyl (meth)acrylamide, N-methyl (meth)acrylamide, N-ethyl (meth)acrylamide, N-propyl (meth)acrylamide, N-butyl (meth)acrylamide, N-hydroxyethyl (meth)acrylamide, N-heptyl (meth)acrylamide, N-octyl (meth)acrylamide, N-ethyhexyl (meth)acrylamide, N-hydroxyethyl (meth)acrylamidecyclohexyl, N-benzyl (meth)acrylamide, N-phenyl (meth)acrylamide, N-tolyl (meth)acrylamide, N-hydroxyphenyl (meth)acrylamide, N-naphthyl (meth)acrylamide, N-phenylsulfonyl (meth)acrylamide, N-methylphenylsulfonyl (meth)acrylamide and N-(meth)acryloylmorpholine; acetal ester or ketal ester compounds, such as norbornene, 2,3-bis(trimethylsilyloxycarbonyl)-5-norbornene, 2,3-bis(triethylsilyloxycarbonyl)-5-norbornene, 2,3-bis(t-butyldimethylsilyloxycarbonyl)-5-norbornene, 2,3-bis(trimethylgermyloxycarbonyl)-5-norbornene, 2,3-bis(triethylgermyloxycarbonyl)-5-norbornene, 2,3-bis(t-butyldimethylgermyloxycarbonyl)-5-norbornene, 2,3-bis(t-butyloxycarbonyl)-5-norbornene, 2,3-bis(benzyloxycarbonryl)-5-norbornene, 2,3-bis(tetrahydrofurane-2-yloxycarbonyl)-5-norbornene, 2,3-bis(cyclopentyloxycarbonyl)-5-norbornene, 2,3-bis(cyclohexyloxycarbonyl)-5-norbornene, 2,3-bis(cycloheptyloxycarbonyl)-5-norbornene, 2,3-bis(1-methoxyethoxycarbonyl)-5-norbornene, 2,3-bis(1-t-butoxyethoxycarbonyl)-5-norbornene, 2,3-bis(1-benzyloxyethoxycarbonyl)-5-norbornene, 2,3-bis[(cyclohexyl)(ethoxy)methoxycarbonyl]-5-norbornene, 2,3-bis(1-methyl-1-methoxyethoxycarbonyl)-5-norbornene, 2,3-bis(1-methyl-1-i-butoxyethoxycarbonyl)-5-norbornene, 2,3-bis[(benzyl)(ethoxy)methoxycarbonyl]-5-norbornene; 1-alkylcycloalkylester compounds, such as 1-metylcyclopropyl (meth)acrylate, 1-methylcyclobutyl (meth)acrylate, 1-methylcyclopentyl (meth)acrylate, 1-methylcyclohexyl (meth)acrylate, 1-methylcycloheptyl (meth)acrylate, 1-methylcyclooctyl (meth)acrylate, 1-methylcyclononyl (meth)acrylate, 1-ethylcyclodecyl (meth)acrylate, 1-ethylcyclopropyl (meth)acrylate, 1-ethylcyclobutyl (meth)acrylate, 1-ethylcyclopentyl (meth)acrylate, 1-ethylcyclohexyl (meth)acrylate, 1-ethylcycloheptyl (meth)acrylate, 1-ethylcyclooctyl (meth)acrylate, 1-ethylcyclononyl (meth)acrylate, 1-ethylcyclodecyl (meth)acrylate, 1-i-propylcyclopropyl (meth)acrylate, 1-i-propylcyclopentyl (meth)acrylate, 1-i-propylcyclohexyl (meth)acrylate, 1-i-propylcycloheptyl (meth)acrylate, 1-i-propylcyclooctyl (meth)acrylate, 1-i-propylcyclononyl (meth)acrylate, 1-i-propylcyclodecyl (meth)acrylate, 1-i-butylcyclopropyl (meth)acrylate, 1-i-butylcyclobutyl (meth)acrylate, 1-i-butylcyclopentyl (meth)acrylate, 1-i-butylcyclohexyl (meth)acrylate, 1-i-butylcyclooctyl (meth)acrylate, 1-i-butylcyclononyl (meth)acrylate, 1-i-butylcyclodecyl (meth)acrylate, 1-i-pentylcyclopropyl (meth)acrylate, 1-i-pentylcyclopentyl (meth)acrylate, 1-i-pentylcyclohexyl (meth)acrylate, 1-i-pentylcycloheptanyl (meth)acrylate, 1-i-pentylcyclooctyl (meth)acrylate, 1-i-pentylcyclononyl (meth)acrylate, 1-i-pentylcyclodecyl (meth)acrylate, 1-i-octylcyclopropyl (meth)acrylate, 1-i-octylcyclobutyl (meth)acrylate, 1-i-octylcycloheptyl (meth)acrylate, 1-i-octylcyclooctyl (meth)acrylate, 1-i-octylcyclononyl (meth)acrylate, 1-i-octylcyclodecyl (meth)acrylate; methacryl acids, such as 3-(methacryloyloxymethyl)oxetane, 3-(methacryloyloxymethyl)-3-ethyloxetane, 3-(methacryloyloxymethyl)-2-methyloxetane, 3-(methacryloyloxymethyl)-2-trifluoromethyloxetane, 3-(methacryloyloxynethyl)-2-pentafluoroethyloxetane, 3-(methacryloyloxymethyl)-2-phenyloxetane, 3-(methacryloyloxymethyl)-2,2-difluorooxetane, 3-(methacryloyloxymethyl)-2,2,4,-trifluorooxetane, 3-(methacryloyloxymethyl)-2,2,4,4-tetrafluorooxetane, 3-(methacryloyloxyethyl)oxetane, 3-(methacryloyloxyethyl)-3-ethyloxeta ne, 2-ethyl-3-(methacryloyloxyethyl)oxetane, 3-(methacryloyloxyethyl)-2-trifluoromethyloxetane, 3-(methacryloyloxyethyl)-2-pentafluoroethyloxetane, 3-(methacryloyloxyethyl)-2-phenyloxetane, 2,2-difluoro-3-(methacryloyloxyethyl)oxetane, 3-(methacryloyloxyethyl)-2,2,4-trifluorooxetane, 3-(methacryloyloxyethyl)-2,2,4,4,-tetrafluorooxetane; polycyclic compounds or anhydride, such as 5-carboxpicyclo[2.2.1]hept-2-ene, 5,6-dicarboxybicyclo[2.2.1]hept-2-ene, 5-carboxy-5-methylbicyclo[2.2.1]hept-2-ene, 5-carboxy-6-ethylbicyclo[2.2.1]hept-2-ene, 5-carboxy-6-methylbicyclo[2.2.1]hept-2-ene, 5-carboxy-6-ethylbicyclo[2.2.1]hept-2-ene, 5,6-dicarboxybicyclo[2.2.1]hept-2-ene anhydride; vinyl or allyl esters, such as vinyl acetate, vinyl propionate, vinyl butylate, vinyl pivalate, vinyl benzoate, vinyl trimethylacetate, vinyl diethylacetate, vinyl borate, vinyl caproate, vinyl chloroacetate, vinyl dichloroacetate, vinyl methoxyacetate, vinyl butoxyacetate, vinyl phenylacetate, vinyl acetate, vinyl acetoacetate, vinyl lactate, vinyl phenylbutylate, vinyl cyclohexylcarboxylate, vinyl salicylate, vinyl chlorobenzoate, vinyl tetrachlorobenzoate, vinyl naphthoate, vinyl triethoxysilane, allyl acetate, allyl propionate, allyl butylate, allyl pivalate, allyl benzoate, allyl caproate, allyl stearate, allyl acetoacetate, allyl lactate; vinyl or allyl ethers, such as vinyl methyl ether, vinyl ethyl ether, vinyl hexyl ether, vinyl octyl ether, vinyl ethylhexyl ether, vinyl methoxyethyl ether, vinyl ethoxyethyl ether, vinyl chloroethyl ether, vinyl hydroxyethyl ether, vinyl ethybutyl ether, vinyl hydroxyethoxyethyl ether, vinyl dimethylaminoethyl ether, vinyl diethylaminoethyl ether, vinyl butylaminoethyl ether, [(ethenyloxy)methyl]triethylsilane, vinyl benzyl ether, vinyl tetrahydrofurfuryl ether, vinyl phenyl ether, vinyl tolyl ether, vinyl chlorophenyl ether, vinyl chloroethyl ether, vinyl dichlorophenyl ether, vinyl naphthyl ether, vinyl anthryl ether, allyl glycidyl ether; crotonates, such as butyl crotonate, hexyl crotonate, glycerine monocrotonate; itaconates, such as dimethyl itaconate, diethyl itaconate, dibutyl itaconate; and maleates or fumarates, such as dimethyl maleate, dibutyl fumarate; polyolefin type compounds, such as butadiene, isoprene, chloroprene and the like; methacrylonitrile, methyl isopropenyl ketone, vinyl acetate, vinyl propionate, vinyl pivalate, maleimide, N-phenylmaleimide, N-methylphenylmaleimide, N-methoxyphenylmaleimide, N-cyclohexylmaleimide, N-alkylmaleimide, maleic anhydride, polystyrene macromonomer, polymethyl (meth)acrylate macromonomer, polybutyl (meth)acrylate macromonomer. Examples of copolymers are copolymers of acrylates and methacrylates with acrylic acid or methacrylic acid and with styrene or substituted styrene, phenolic resins, for example novolak, (poly)hydroxystyrene, and copolymers of hydroxystyrene with alkyl acrylates, acrylic acid and/or methacrylic acid. Preferable examples of copolymers are copolymers of methyl (meth)acrylate/(meth)acrylic acid, copolymers of benzyl (meth)acrylate/(meth)acrylic acid, copolymers of methyl (meth)acrylate/ethyl (meth)acrylate/(meth)acrylic acid, copolymers of benzyl (meth)acrylate/(meth)acrylic acid/styrene, copolymers of benzyl (meth)acrylate/(meth)acrylic acid/hydroxyethyl (meth)acrylate, copolymers of benzyl (meth)acrylate/(meth)acrylic acid/glycidyl (meth)acrylate, copolymers of benzyl (meth)acrylate/(meth)acrylic acid/3-(methacryloyloxymethyl)oxetane, copolymers of methyl (meth)acrylate/butyl (meth)acrylate/(meth)acrylic acid/styrene, copolymers of methyl (meth)acrylate/benzyl (meth)acrylate/(meth)acrylic acid/hydroxyphenyl (meth)acrylate, copolymers of methyl (meth)acrylate/(meth)acrylic acid/polymethyl (meth)acrylate macromonomer, copolymers of benzyl (meth)acrylate/(meth)acrylic acid/polymethyl (meth)acrylate macromonomer, copolymers of tetrahydrofurfuryl (meth)acrylate/styrene/(meth)acrylic acid, copolymers of methyl (meth)acrylate/(meth)acrylic acid/polystyrene macromonomer, copolymers of benzyl (meth)acrylate/(meth)acrylic acid/polystyrene macromonomer, copolymers of benzyl (meth)acrylate/(meth)acrylic acid/2-hydroxyethyl (meth)acrylate/polystyrene macromonomer, copolymers of benzyl (meth)acrylate/(meth)acrylic acid/2-hydroxypropyl (meth)acrylate/polystyrene macromonomer, copolymers of benzyl (meth)acrylate/(meth)acrylic acid/2-hydroxy-3-phenoxypropyl (meth)acrylate/polymethyl (meth)acrylate macromonomer, copolymers of methyl (meth)acrylate/(meth)acrylic acid/2-hydroxyethyl (meth)acrylate/polystyrene macromonomer, copolymers of benzyl (metha)crylate/(meth)acrylic acid/2-hydroxyethyl (meth)acrylate/polymethyl (meth)acrylate macromonomer, copolymers of N-phenylmaleimide/benzyl (meth)acrylate/(meth)acrylic acid and styrene, copolymers of benzyl (meth)acrylate/(meth)acrylic acid/N-phenylmaleimide/mono-[2-(meth)acryloyloxyethyl] succinate/styrene, copolymers of allyl (meth)acrylate/(meth)acrylic acid/N-phenyl-maleimide/mono-[2-(meth)acryloyloxyethyl] succinate/styrene, copolymers of benzyl (meth)acrylate/(meth)acrylic acid/N-phenylmaleimide/glycerol mono(meth)acrylate/styrene, copolymers of benzyl (meth)acrylateko-carboxypolycaprolactone mono(meth)acrylate/(meth)acrylic acid/N-phenylmaleimide/glycerol mono(meth)acrylate/styrene, and copolymers of benzyl (meth)acrylate/(meth)acrylic acid/N-cyclohexylmaleimide/styrene. Example of commercial product is Ripoxy SPC-2000 provided by Showa Denko K.K. The term “(meth)acrylate” in the context of the present application is meant to refer to the acrylate as well as to the corresponding methacrylate.

One or more monomers selected from the group consisting of silicon containing monomers such as silanes, such as tetraethylorthosilicate or tetraethoxysilane or chloro- or alkoxy functional silanes, olefins such as ethylene, propylene, styrene, vinylpyrrolidone, oxygen- or nitrogen-containing monomers such as acrylic derivatives, e.g. acrylic ester and acrylic acid, methacrylic acid and -ester, urethanes, mono- and di-functional alcohols, carboxylic acids, amines, isocyanates, epoxides, aromatic compounds such as aromatics carrying substituents such as alkyl groups and sulfonated aromatics, aromatic resins, imidazole and imidazole derivatives, pyrazoles, quartenary ammonium compounds, polyurethane prepolymers and epoxy resins The functional groups in the compound, which result in good alkaline solubility, are preferably carboxylic groups. However, also other groups, which result in alkaline solubility, are possible. Examples for such groups are phenolic groups, sulfonic acid groups and anhydride groups

When the number of ethylenically unsaturated bonds, which are present in the molecular unit of the resin curable by the activated energy ray, is small, it is possible to use bis-phenol A type epoxy compounds to lower the viscosity of the ink. The novolak type epoxy compounds are represented by phenol novolak type epoxy resins and cresol novolak type epoxy resins. Such compounds are typically produced by reacting epichlorohydrin with a novolak resin.

Typical examples of the aforementioned acid anhydride are dibasic acid anhydrides such as for example maleic anhydride, succinic anhydride, itaconic anhydride, phthalic anhydride, tetrahydrophthalic anhydride, hexahydrophthalic anhydride, methylhexahydrophthalic anhydride, endo-methylenetetrahydrophthalic anhydride, methyl-endo-methylenetetrahydrophthalic anhydride, chlorendic anhydride, and methyltetrahydrophthalic anhydride; aromatic polycarboxylic anhydrides such as for example trimellitic anhydride, pyromellitic anhydride and benzophenone-tetracarboxylic dianhydride: and polycarboxylic anhydride derivatives such as 5-(2,5-dioxotetrahydrofuryl)-3-methyl-3-cyclohexene-1, 2-dicarboxylic anhydride.

Further examples of alkaline developable resins (a) are polymers or oligomers having at least two ethylenically unsaturated groups and at least one carboxyl function within the molecule structure, such as a resin obtained by the reaction of a saturated or unsaturated polybasic acid anhydride with a product of the reaction of an epoxy compound and an unsaturated monocarboxylic acid (for example, EB9696 from UCB Chemicals; KAYARAD TCR1025 from Nippon Kayaku Co. Ltd.; NK OLIGO EA-6340, EA-7440 from Shin-Nakamura Chemical Co., Ltd.). Other examples of such binders are described in JP2002-206014A, JP2004-69754A, JP2004-302245A, JP2005-77451A, JP2005-316449A, JP2005-338328A and JP3754065B2.

Further examples of alkaline developable resins (a) are the above-mentioned polymers or oligomers having at least one ethylenically unsaturated groups.

Further examples are reaction products obtained by adding an epoxy group containing unsaturated compound to a part of the carboxyl groups of a carboxylic acid group containing polymer (for ex., ACA200, ACA200M, ACA210P, ACA230AA, ACA250, ACA300, ACA320 from Daicel Co. and Ripoxy SPC-1000 provided by Showa Denko K. K.). As the carboxylic acid containing polymer, the abovementioned binder polymers which are resulting from the reaction of an unsaturated carboxylic acid compound with one or more polymerizable compounds, for example, copolymers of (meth)acrylic acid, benzyl (meth)acrylate, styrene and 2-hydroxyethyl (meth)acrylate, copolymers of (meth)acrylic acid, styrene and α-methylstyrene, copolymers of (meth)acrylic acid, N-phenylmaleimide, styrene and benzyl (meth)acrylate, copolymers of (meth)acrylic acid and styrene, copolymers of (meth)acrylic acid and benzyl (meth)acrylate, copolymers of tetrahydrofurfuryl (meth)acrylate, styrene and (meth)acrylic acid and the like. Examples of the unsaturated compounds having an epoxy group are given below in the formula (V-1)-(V-15);

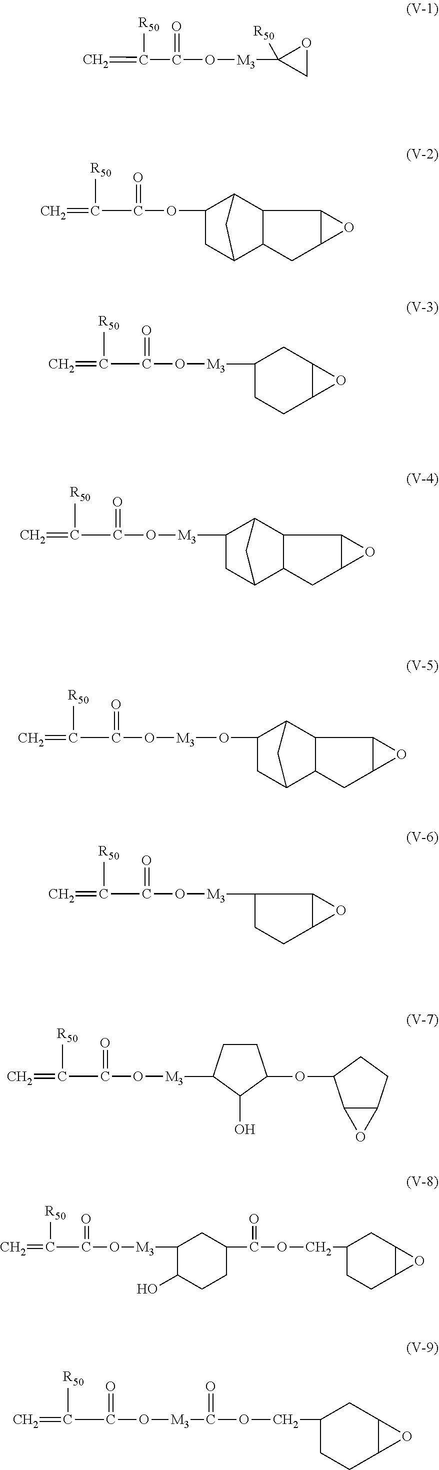

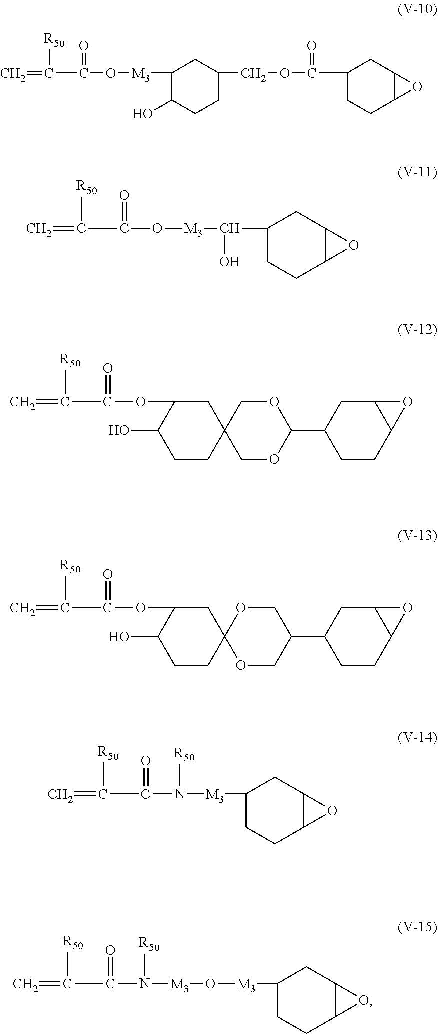

wherein R₅₀ is hydrogen or methyl group, M₃ is substituted or unsubstituted alkylene having 1 to 10 carbon atoms.

Among these compounds, compounds having alicyclic epoxy groups are particularly preferred, because these compounds have a high reactivity with carboxyl group-containing resins, accordingly the reaction time can be shortened. These compounds further do not cause gelation in the process of reaction and make it possible to carry out the reaction stably. On the other hand, glycidyl acrylate and glycidyl methacrylate are advantageous from the viewpoint of sensitivity and heat resistance because they have a low molecular weight and can give a high conversion of esterification.

Concrete examples of the abovementioned compounds are, for example a reaction product of a copolymer of styrene, α-methylstyrene and acrylic acid or a copolymer of methyl methacrylate and acrylic acid with 3,4-epoxycyclohexylmethyl (meth)acrylate.

Other examples are products obtained by addition reaction of an epoxy group containing unsaturated compounds to a part of or all of the carboxyl groups of a carboxylic acid group containing polymers followed by further reaction with polybasic acid anhydride (for ex., Ripoxy SPC-3000 provided by Showa Denko K.K.).

Unsaturated compounds having a hydroxy group such as 2-hydroxyethyl (meth)acrylate and glycerol mono(meth)acrylate can be used instead of the abovementioned epoxy group containing unsaturated compounds as the reactant for carboxylic acid group containing polymers.

Other examples are half esters of anhydride containing polymers, for example reaction products of a copolymer of maleic anhydride and one or more other polymerizable compounds with (meth)acrylates having an alcoholic hydroxyl group such as 2-hydroxyethyl (meth)acrylate or having an epoxy group for example such as the compounds described in the formula (V-1)-(V-15).

Reaction products of polymers having alcoholic hydroxyl groups such as copolymers of 2-hydroxyethyl (meth)acrylate, (meth)acrylic acid, benzyl (meth)acylate and styrene, with (meth)acrylic acid or (meth)acryl chloride can also be used.

Other examples are reaction products of polyester with terminal unsaturated groups, which is obtained from the reaction of a dibasic acid anhydride and a compound having at least two epoxy groups followed by further reaction with an unsaturated compound, with a polybasic acid anhydride.

Further examples are resins obtained by the reaction of a saturated or unsaturated polybasic acid anhydride with a reaction product obtained by adding epoxy groups containing (meth)acrylic compounds to all of the carboxyl groups of a carboxylic acid containing polymer as mentioned above.

Other example is polyimide resin having ethylenically unsaturated groups and at least one carboxyl function. The polyimide binder resin in the present invention can be a polyimide precursor, for example, a poly(amic acid).

Specific examples of alkaline developable resins are:

Acrylpolymer type resins such as

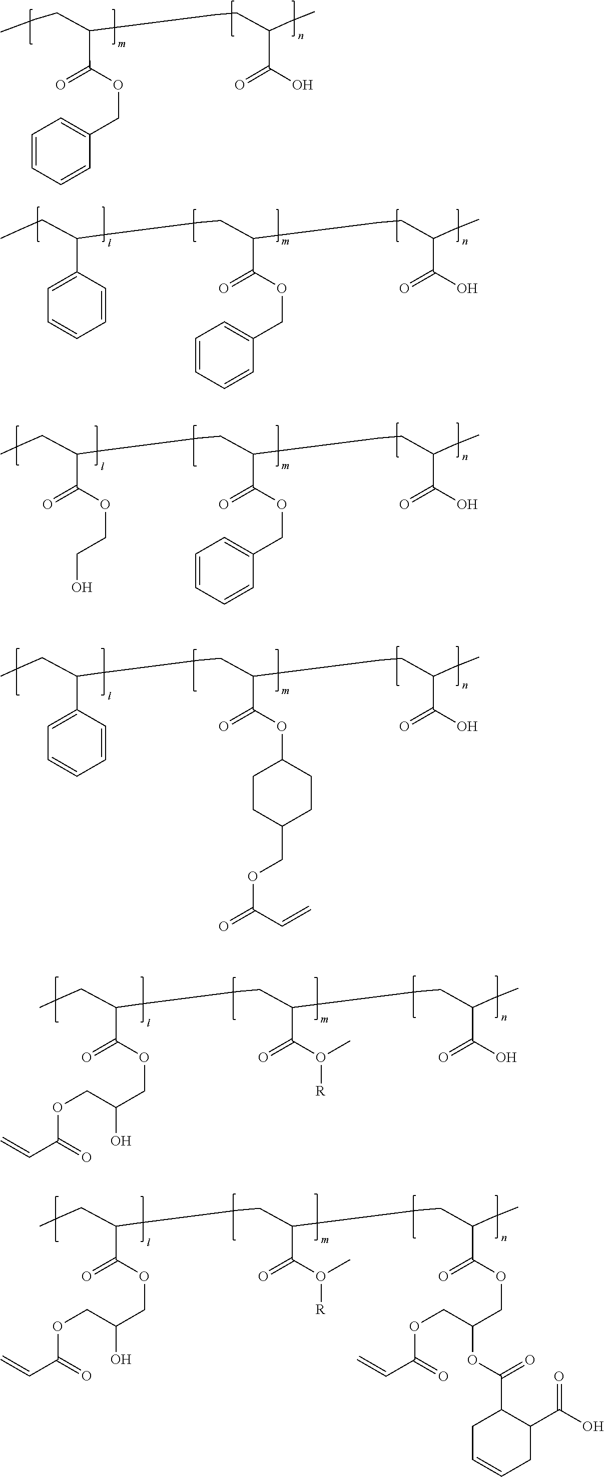

Cardo Type Resin (Fluorene Epoxy Acrylate Based Resin)

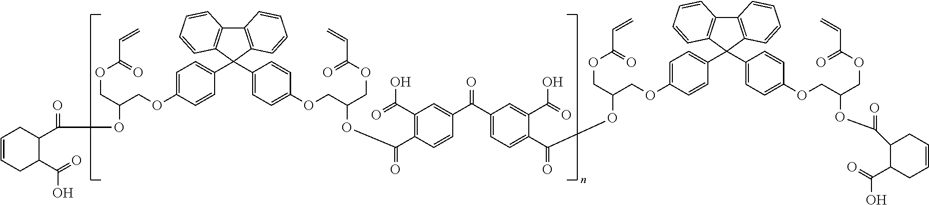

For example, a content of the binder resin may be 2-98%, preferably 5-90% and especially 10-80% by weight based on a total weight of the solid contents in the radically polymerizable composition.

Component (a) for example comprises an acrylate monomer.

The acrylate monomer refers to an acrylate monomer or oligomer that contains one or more acryloyl or methacryloyl moieties or combinations thereof.

Examples of compounds containing a double bond are (meth)acrylic acid, alkyl, hydroxyalkyl or aminoalkyl (meth)acrylates, for example methyl, ethyl, n-butyl, i-butyl, t-butyl, n-propyl, i-propyl, n-hexyl, cyclohexyl, 2-ethylhexyl, isobornyl, benzyl, 2-hydroxyethyl, 2-hydroxypropyl, methoxyethyl, ethoxyethyl, glycerol, phenoxyethyl, methoxydiethylene glycol, ethoxydiethylene glycol, polyethylene glycol, polypropylene glycol, glycidyl, N, N-dimethylaminoethyl, and N, N-diethylaminoethyl (meth)acrylates. Other examples are (meth)acrylonitrile, (meth)acrylamide, N-substituted (meth)acrylamides such as N, N-dimethyl (meth)acrylamide, N, N-diethyl (meth)acrylamide, N, N-dibutyl (meth)acrylamide, N-methyl (meth)acrylamide, N-ethyl (meth)acrylamide, N-butyl (meth)acrylamide, and N-(meth)acryloylmorpholine, vinyl esters such as vinyl acetate, vinyl ethers such as i-butyl vinyl ether, styrene, alkyl-, hydroxy- and halostyrenes, N-vinylpyrrolidone, N-vinylcaprolactam, N-vinyl acetoamide, N-vinyl formamide, vinyl chloride and vinylidene chloride.

Examples of polyunsaturated compounds of relatively high molecular mass (oligomers) are polyesters, polyurethanes, polyethers and polyamides, which contain ethylenically unsaturated carboxylates.

Particularly suitable examples are esters of an ethylenically unsaturated carboxylic acid with a polyol or polyepoxide.

Examples of unsaturated carboxylic acids are acrylic acid, methacrylic acid, crotonic acid, itaconic acid, cinnamic acid, and unsaturated fatty acids such as linolenic acid or oleic acid. Acrylic and methacrylic acids are preferred.

Suitable polyols are aromatic, in particular, aliphatic and cycloaliphatic polyols. Examples of aromatic polyols are hydroquinone, 4,4′-dihydroxybiphenyl, 2,2-bis(4-hydroxyphenyl)ethane, 2,2-bis(4-hydroxyphenyl)propane, 2,2-bis(4-hydroxyphenyl)hexafluoropropane, 9,9-bis(4-hydroxyphenyl)fluorene, novolacs and resols. Examples of aliphatic and cycloaliphatic polyols are alkylenediols having preferably 2 to 12 carbon atoms, such as ethylene glycol, 1,2- or 1,3-propanediol, 1,2-, 1,3- or 1,4-butanediol, pentanediol, hexanediol, octanediol, dodecanediol, diethylene glycol, triethylene glcyol, polyethylene glycols having molecular weights of preferably from 200 to 1500, 1,3-cyclopentanediol, 1,2-, 1,3- or 1,4-cyclohexanediol, 1,4-dihydroxymethylcyclohexane, glycerol, triethanolamine, trimethylolethane, trimethylolpropane, pentaerythritol, pentaerythritol monooxalate, dipentaerythritol, ethers of pentaerythritol with ethylene glycol or propylene glycol, ethers of dipentaerythritol with ethylene glycol or propylene glycol, sorbitol, 2,2-bis[4-(2-hydroxyethoxy)phenyl]methane, 2,2-bis[4-(2-hydroxyethoxy)phenyl]propane and 9,9-bis[4-(2-hydroxyethoxy)phenyl]fluorene. Other suitable polyols are polymers and copolymers containing hydroxyl groups in the polymer chain or in side groups, examples being homopolymers or copolymers comprising vinyl alcohol or comprising hydroxyalkyl (meth)acrylates. Further polyols which are suitable are esters and urethanes having hydroxyl end groups.

The polyols may be partially or completely esterified with one unsaturated carboxylic acid or with different unsaturated carboxylic acids, and in partial esters the free hydroxyl groups may be modified, for example etherified or esterified with other carboxylic acids.

Examples of esters based on polyols are trimethylolpropane tri(meth)acrylate, trimethylolpropane tri(acryloyloxypropyl) ether, trimethylolethane tri(meth)acrylate, ethylene glycol di(meth)acrylate, diethylene glycol di(meth)acrylate, triethylene glycol di(meth)acrylate, tetraethylene glycol di(meth)acrylate, tetramethylene glycol di(meth)acrylate, neopentyl glycol di(meth)acrylate, pentaerythritol di(meth)acrylate, pentaerythritol tri(meth)acrylate, pentaerythritol tetra(meth)acrylate, pentaerythritol tri(meth)acrylate monooxalate, dipentaerythritol di(meth)acrylate, dipentaerythritol tri(meth)acrylate, dipentaerythritol tetra(meth)acrylate, dipentaerythritol penta(meth)acrylate, dipentaerythritol hexa(meth)acrylate, dipentaerythritol penta(meth)acrylate mono(2-hydroxyethyl) ether, tripentaerythritol octa(meth)acrylate, 1,3-butanediol di(meth)acrylate, 1,4-butanediol diitaconate, hexanediol di(meth)acrylate, 1,4-cyclohexanediol di(meth)acrylate, sorbitol tri(meth)acrylate, sorbitol tetra(meth)acrylate, sorbitol penta(meth)acrylate, sorbitol hexa(meth)acrylate, oligoester (meth)acrylates, glycerol di(meth)acrylate and tri(meth)acrylate, di(meth)acrylates of polyethylene glycol with a molecular weight of from 200 to 1500, pentaerythritol diitaconate, dipentaerythritol trisitaconate, dipentaerythritol pentaitaconate, dipentaerythritol hexaitaconate, ethylene glycol diitaconate, propylene glycol diitaconate, 1,3-butanediol diitaconate, 1,4-butanediol diitaconate, tetramethylene glycol diitaconate, sorbitol tetraitaconate, ethylene glycol dicrotonate, tetramethylene glycol dicrotonate, pentaerythritol dicrotonate, ethylene glycol dimaleate, tiethylene glycol dimaleate, pentaerythritol dimaleate, sorbitol tetramaleate, or mixtures thereof.

Other examples are pentaerythritol and dipentaerythritol derivatives shown in the following formula (XII) and (XIII):

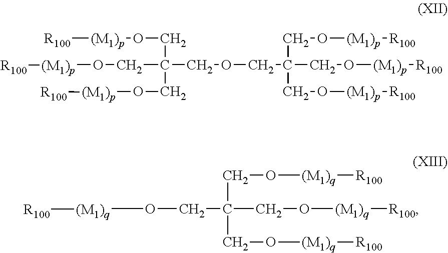

wherein

M₁ is —(CH₂CH₂O)— or —[CH₂CH(CH₃)O]—,

R₁₀₀ is —COCH═CH₂ or —COC(CH₃)═CH₂, p is 0 to 6 (total of p: 3-24), and q is 0 to 6 (total of q: 2-16).

Examples of polyepoxides are those based on the abovementioned polyols and epichlorohydrin. Typical examples are bis(4-glycidyloxyphenyl)methane, 2,2-bis(4-glycidyloxyphenyl)propane, 2,2-bis(4-glycidyloxyphenyl)hexafluoropropane, 9,9-bis(4-glycidyloxyphenyl)fluorene, bis[4-(2-glycidyloxyethoxy)phenyl]methane, 2,2-bis[4-(2-glycidyloxyethoxy)phenyl]propane, 2,2-bis[4-(2-glycidyloxyethoxy)phenyl]hexafluoropropane, 9,9-bis[4-(2-glycidyloxyethoxy)phenyl]fluorene, bis[4-(2-glycidyloxypropoxy)phenyl]methane, 2,2-bis[4-(2-glycidyloxypropoxy)phenyl]propane, 2,2-bis[4-(2-glycidyloxypropoxy)phenyl]hexafluoropropane, 9,9-bis[4-(2-glycidyloxypropoxy)phenyl]fluorene, glycerol diglycidyl ether and glycidyl ethers of phenol and cresol novolacs.

Typical examples based on polyepoxides are 2,2-bis[4-{(2-hydroxy-3-acryloxy)propoxy}phenyl]propane, 2,2-bis[4-{(2-hydroxy-3-acryloxy)propoxyethoxy}phenyl]propane, 9,9-bis[4-{(2-hydroxy-3-acryloxy)propoxy}phenyl]fluorene, 9,9-bis[4-{(2-hydroxy-3-acryl-wry)propoxyethoxy}phenyl]fluorene, glycerol 1,3-diglycerolate diacrylate and reaction products of epoxy resins based on novolacs with (meth)acrylic acid.

Preferred multifunctional (meth)acrylate monomers or oligomers include pentaerythritol tetraacrylate, dipentaerythritol pentaacrylate, dipentaerythritol hexaacrylate, di-trimethylolpropane tetraacrylate, pentaerythritol triacrylate, tris(2-hydroxy ethyl) isocyanurate triacrylate.

Specific examples are:

Dipentaerythritol-Hexaacrylate (DPHA)

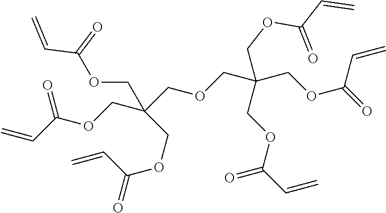 [SMILES: C=CC(=O)OCC(COCC(COC(=O)C=C)(COC(=O)C=C)COC(=O)C=C)(COC(=O)C=C)COC(=O)C=C]

Dipentaerythritol-Pentaacrylate (DPPA)

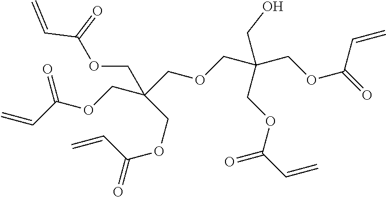 [SMILES: C=CC(=O)OCC(CO)(COCC(COC(=O)C=C)(COC(=O)C=C)COC(=O)C=C)COC(=O)C=C]

Examples of commercially available acrylate monomer or oligomers having two acryloyl or methacryloyl moieties are Aronix®M-210, Aronix®M-240, Aronix®M-6200 (TOAGOSEI Co., LDT.) KAYARAD HDDA, KAYARAD HX-220, KAYARAD HX-620, KAYARAD R-526, KAYARAD UX-2201, KAYARAD MU-2100 (NIPPON KAYAKU Co., LTD.), VISCOAT-260, VISCOAT-355HP (OSAKA ORGANIC CHEMICAL INDUSTRY LTD.).

Examples of commercially available acrylate monomer or oligomers having three or more acryloyl or methacryloyl moieties are Aronix®M-309, Aronix®M-400, Aronix®M-1310, Aronix®M-1960, Aronix®M-7100, Aronix®M-8530, Aronix®TO-1450 (TOAGOSEI Co., LDT.), KAYARAD TMPTA, KAYARAD DPHA, KAYARAD DPCA-20, KAYARAD MAX-3510 (NIPPON KAYAKU Co., LTD.), VISCOAT-295, VISCOAT-300, VISCOAT-GPT, VISCOAT-3PA, VISCOAT-400 (OSAKA ORGANIC CHEMICAL INDUSTRY LTD.).

Examples of commercially available urethane acrylate monomer or oligomers having two or more acryloyl or methacryloyl moieties are NEW FRONTIER R-1150 (DAI-ICHI KOGYO SEIYAKU CO., LTD.) KAYARAD DPHA-40H, KAYARAD UX-5000 (NIPPON KAYAKU Co., LTD.), UN-9000H (Negami Chemical Industrial Co., Ltd.)

The amount of acrylate present in the radiation curable composition ranges from about 2% to 80% and preferably from about 5% to 70% based on the whole solid contents of the composition, i.e. the amount of all components without the solvent(s).

It is of course possible to add other known photoinitiators (c) to the photocurable composition. The use of the further photoinitiator is not critical. The photoinitiator (c) is for example selected from benzophenones, bis-imidazole, aromatic α-hydroxyketones, benzylketals, aromatic α-aminoketones, phenylglyoxalic acid esters, mono-acylphosphinoxides, bis-acylphosphinoxides, tris-acylphosphinoxides, oximesters derived from aromatic ketones and/or oxime esters of the carbazol type.

Examples of photoinitiators are camphor quinone; benzophenone, benzophenone derivatives, such as 2,4,6-trimethylbenzophenone, 2-methylbenzophenone, 3-methylbenzophenone, 4-methylbenzophenone, 2-methoxycarbonylbenzophenone, 4,4′-bis(chloromethyl)benzophenone, 4-chlorobenzophenone, 4-phenylbenzophenone, 3,3′-dimethyl-4-methoxy-benzophenone, [4-(4-methylphenylthio)phenyl]-phenylmethanone, methyl-2-benzoylbenzoate, 3-methyl-4′-phenylbenzophenone, 2,4,6-trimethyl-4′-phenylbenzophenone, 4,4′-bis(dimethylamino)benzophenone, 4,4′-bis(diethylamino)benzophenone, thioxanthones, thioxanthone derivatives, polymeric thioxanthones as for example OMNIPOL TX; ketal compounds, as for example benzildimethylketal (IRGACURE® 651); acetophenone, acetophenone derivatives, for example α-hydroxycycloalkyl phenyl ketones or α-hydroxyalkyl phenyl ketones, such as for example 2-hydroxy-2-methyl-1-phenyl-propanone (DAROCURE01173), 1-hydroxy-cyclohexyl-phenyl-ketone (IRGACURE0184), 1-(4-dodecylbenzoyl)-1-hydroxy-1-methyl-ethane, 1-(4-i-propylbenzoyl)-1-hydroxy-1-methyl-ethane, 1-[4-(2-hydroxyethoxy)-phenyl]-2-hydroxy-2-methyl-1-propan-1-one (IRGACURE®2959); 2-hydroxy-1-{4-[4-(2-hydroxy-2-methyl-propionyl)-benzyl]phenyl}-2-methyl-propan-1-one (IRGACURE® 127); 2-hydroxy-1-{4-[4-(2-hydroxy-2-methyl-propionyl)-phenoxy]-phenyl}-2-methyl-propan-1-one; dialkoxyacetophenones, α-hydroxy- or α-aminoacetophenones, e.g. (4-methylthiobenzoyl)-1-methyl-1-morpholinoethane (IRGACURE® 907), (2-Benzyl-2-(dimethylamino)-1-[4-(4-morpholinyl)phenyl]-1-butanone) (IRGACURE® 369), 2-(4-Methylbenzyl)-2-(dimethylamino)-1-[4-(4-morpholinyl)phenyl]-1-butanone (IRGACURE® 379), (4-(2-hydroxyethyl)aminobenzoyl)-1-benzyl-1-dimethylaminopropane), (3,4-dimethoxpenzoyl)-1-benzyl-1-dimethylaminopropane; 1-[4-[4-(2-hydroxyethoxy)phenyl]sulfanylphenyl]-2-methyl-2-morpholino-propan-1-one, 4-aroyl-1,3-dioxolanes, benzoin alkyl ethers and benzyl ketals, e.g. dimethyl benzyl ketal, phenylglyoxalic esters and derivatives thereof, e.g. methyl α-oxo benzeneacetate, oxo-phenyl-acetic acid 2-(2-hydroxy-ethoxy)-ethyl ester, dimeric phenylglyoxalic esters, e.g. oxo-phenyl-acetic acid 1-methyl-2-[2-(2-oxo-2-phenyl-acetoxy)-propoxy]ethyl ester (IRGACURE® 754); ketosulfones, e.g. ESACURE KIP 1001 M; oximeesters, e.g. 1,2-octanedione 1-[4-(phenylthio)phenyl]-2-(O-benzoyloxime) (IRGACURE® OXE01), ethanone 1-[9-ethyl-6-(2-methylbenzoyl)-9H-carbazol-3-yl]-1-(O-acetyloxime) (IRGACURE® OXE02), Methanone, [8-[[acetyloxy)imino][2-(2,2,3,3-tetrafluoropropoxy)phenyl]methyl]-11-(2-ethylhexyl)-11H-benzo[a]carbazol-5-yl](2,4,6-trimethylphenyl), [1-[4-[4-(benzofuran-2-carbonyl)phenyl]sulfanylphenyl]-4-methyl-pentylidene]amino] acetate, ethanone 1-[9-ethyl-6-(2-methyl-4-(2,2-dimethyl-1,3-dioxolanyl)methoxybenzoyl)-9H-carbazol-3-yl]-1-(O-acetyloxime), [3-cyclopentyl-1-[9-ethyl-6-(thiophene-2-carbonyl)carbazol-3-yl]propylidene]amino] acetate, N-Acetoxy-N-{3-[9-ethyl-6-(naphthalene-1-carbonyl)-9H-carbazol-3-yl]-1-methyl-3-acetoxyimino-propyl}acetamide, 9H-thioxanthene-2-carboxaldehyde 9-oxo-2-(O-acetyloxime), [[1-(cyclohexylmethyl)-2-oxo-2-(4-phenylsulfanylphenyl)ethylidene]amino] cyclopropanecarboxylate), [[1-(cyclohexylmethyl)-2-oxo-2-(4-phenylsulfanylphenyl)ethylidene]amino] acetate, [1-(cyclohexylmethyl)-2-[9-ethyl-6-(thiophene-2-carbonyl)carbazol-3-yl]-2-oxo-ethylidene]amino] acetate, [1-(cyclohexylmethyl)-2-[9-ethyl-6-(furan-2-carbonyl)carbazol-3-yl]-2-oxo-ethylidene]amino] acetate, [1-(o-tolyl)-2-oxo-2-(4-phenylsulfanylphenyl)ethylidene]amino] acetate, 1-[1-(4-benzoylphenyl)indole-3-carbonyl]heptylideneamino] acetate, 1-[9-(4-benzoylphenyl)carbazol-3-yl]propylideneamino] acetate, [5-(4-isopropylphenyl)sulfanyl-1-oxo-indan-2-ylidene]amino] acetate, 1-(9,9-dibutyl-7-nitro-fluoren-2-yl)ethylideneamino] acetate, [2-(9,9-diethylfluoren-2-yl)-1-methyl-2-oxo-ethylidene]amino] acetate, [(7-nitro-9,9-dipropyl-fluoren-2-yl)-(o-tolyl)methylene]amino] acetate, [2-(9,9-dibutylfluoren-2-yl)-1-(o-tolyl)-2-oxo-ethylidene]amino] acetate, the oxime esters described in WO 07/062963, WO 07/071797, WO 07/071497, WO 05/080337, JP2010-049238, WO2008078678, JP2010-15025 and JP2010-49238, peresters, e,g. benzophenone tetracarboxylic peresters as described for example in EP 126541, monoacyl phosphine oxides, e.g. (2,4,6-trimethylbenzoyl)diphenylphosphine oxide (DAROCURE® TPO), ethyl (2,4,6 trimethylbenzoyl phenyl) phosphinic acid ester; bisacylphosphine oxides, e.g. bis(2,6-dimethoxy-benzoyl)-(2,4,4-trimethyl-pentypphosphine oxide, bis(2,4,6-trimethylbenzoyl)-phenylphosphine oxide (IRGACURE® 819), bis(2,4,6-trimethylbenzoyl)-2,4-dipentoxyphenylphosphine oxide, trisacylphosphine oxides, halomethyltriazines, e.g. 2-[2-(4-methoxy-phenyl)-vinyl]-4,6-bis-trichloromethyl-[1,3,5]triazine, 2-(4-methoxy-phenyl)-4,6-bis-trichloromethyl-[1,3,5]triazine, 2-(3,4-dimethoxy-phenyl)-4,6-bis-trichloromethyl-[1,3,5]triazine, 2-methyl-4,6-bis-trichloromethyl-[1,3,5]triazine, hexaarylbisimidazole/coinitiators systems, e.g. ortho-chlorohexaphenyl-bisimidazole combined with 2-mercaptobenzothiazole, ferrocenium compounds, or titanocenes, e.g. bis(cyclopentadienyl)-bis(2,6-difluoro-3-pyrryl-phenyl)titanium (IRGACURE®784). Further, borate compounds can be used as coinitiators. As additional photoinitiators oligomeric compounds such as for example oligomeric alpha hydroxyl ketones, e.g. 2-hydroxy-1-{1-[4-(2-hydroxy-2-methyl-propionyl)-phenyl]-1,3,3-trimethyl-indan-5-yl}-2-methyl-propan-1-one, ESACURE KIP provided by Fratelli Lamberti, or oligomeric alpha amino ketones may be employed as well.

Specific examples are:

IRGACURE®369

### (2-Benzyl-2-(dimethylamino)-1-[4-(4-morpholinyl)phenyl]-1-butanone)

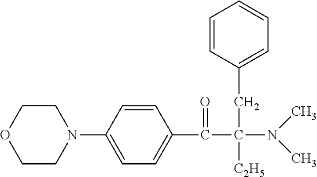 [SMILES: CCC(Cc1ccccc1)(C(=O)c1ccc(N2CCOCC2)cc1)N(C)C]

IRGACURE®379

### 2-(4-Methylbenzyl)-2-(dimethylamino)-1-[4-(4-morpholinyl)phenyl]-1-butanone

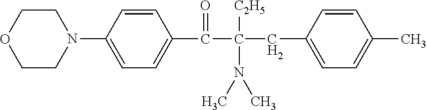 [SMILES: CCC(Cc1ccc(C)cc1)(C(=O)c1ccc(N2CCOCC2)cc1)N(C)C]

IRGACURE®OXE01

### 1,2-Octanedione 1-[4-(phenylthio)phenyl]-2-(O-benzoyloxime)

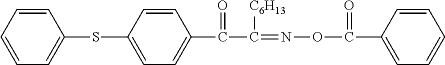

IRGACURE®OXE02

### Ethanone 1-[9-ethyl-6-(2-methylbenzoyl)-9H-carbazol-3-yl]-1-(O-acetyloxime)

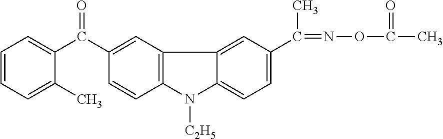 [SMILES: CCn1c2ccc(C(=O)c3ccccc3C)cc2c2cc(C(C)=NOC(C)=O)ccc21]

The composition according to the invention may comprise as component (d) a pigment, or a mixture of pigments, or a dye, or a mixture of dyes, or a mixture of one or more pigments with one or more dyes. The pigments which can be comprised in the composition according to the present invention, including a pigmented color filter resist composition, are preferably processed pigments.

The red pigment comprises, for example, an anthraquinone type pigment alone, a diketopyrolopyrole type pigment alone, a mixture of them or a mixture consisting of at least one of them and a disazo type yellow pigment or an isoindoline type yellow pigment, in particular C. I. Pigment Red 177 alone, C. I. Pigment Red 254 alone, a mixture of C. I. Pigment Red 177 and C. I. Pigment Red 254 or a mixture consisting of at least one member of C. I. Pigment Red 177, C. I. Pigment Red 242 and C. I. Pigment Red 254, and C. I. Pigment Yellow 83 or C. I. Pigment Yellow 139 (“C.I.” refers to the Color Index, known to the person skilled in the art and publicly available).

Further suitable examples for the pigment are C.I. Pigment Red 9, 97, 105, 122, 123, 144, 149, 168, 176, 179, 180, 185, 202, 207, 209, 214, 222, 244, 255, 264, 272 and C.I. Pigment Yellow 12, 13, 14, 17, 20, 24, 31, 53, 55, 93, 95, 109, 110, 128, 129, 138, 139, 150, 153, 154, 155, 166, 168, 185, 199, 213 and C.I. Pigment Orange 43 and 71.

Examples of the dyes for red color are C. I. Solvent Red 25, 27, 30, 35, 49, 83, 89, 100, 122, 138, 149, 150, 160, 179, 218, 230, C. I. Direct Red 20, 37, 39, 44, and C. I. Acid Red 6, 8, 9, 13, 14, 18, 26, 27, 51, 52, 87, 88, 89, 92, 94, 97, 111, 114, 115, 134, 145, 151, 154, 180, 183, 184, 186, 198, C. I. Basic Red 12, 13, C. I. Disperse Red 5, 7, 13, 17 and 58. The Red dyes can be used in combination with yellow and/or orange dyes.

The green pigment comprises for instance a halogenated phthalocyanine type pigment alone or its mixture with a bisazo type yellow pigment, an quinophthalone type yellow pigment or a metal complex, in particular C. I. Pigment Green 7 alone, C. I. Pigment Green 36 alone, C.I. Pigment 58 alone, or a mixture consisting of at least one member of C. I. Pigment Green 7, C. I. Pigment Green 36, C.I. Pigment Green 58, C.I. Pigment Green 59, C.I. Pigment Green 62, C.I. Pigment Green 63 and C. I. Pigment Yellow 83, C. I. Pigment Yellow 138, Pigment Yellow 139 or C. I. Pigment Yellow 150. Other suitable green pigments are C.I. Pigment Green 15, 25 and 37. Examples for suitable green dyes are C. I. Acid Green 3, 9, 16, C. I. Basic Green 1 and 4.

Examples for suitable blue pigments are phthalocyanine type pigments, used either alone or in combination with an dioxazine type violet pigment, for instance, C. I. Pigment Blue 15:6 alone, a combination of C. I. Pigment Blue 15:6 and C. I. Pigment Violet 23. Further examples for blue pigments are such of C. I. Pigment Blue 15:3, 15:4, 16, 22, 28 and 60. Other suitable pigments are C. I. Pigment Violet 14, 19, 23, 29, 32, 37, 177 and C. I. Orange 73.

The blue dye comprises, for example, a methane type dye, an anthraquinone type dye, an azo type dye, a metal complex azo type dye, a triaryl methane type dye or a phthalocyanine type dye. Examples for suitable blue dyes are C. I. Solvent Blue 11, 25, 37, 45, 49, 68, 78, 94, C. I. Direct Blue 25, 86, 90, 108, C. I. Acid Blue 1, 3, 7, 9, 15, 83, 90, 103, 104, 158, 161, 249, C. I. Basic Blue 1, 3, 7, 9, 25, 105, and C. I. Disperse Blue 198 and C.I. Mordant Blue 1.

The pigment of the photopolymeric composition for black matrix preferably comprises at least one member selected from the group consisting of carbon black, titanium black, iron oxide, lactone, lactam and perylene. Preferred example is carbon black. However, a mixture of other pigments which, in total, give the black appearance, can also be used. For example, also C. I. Pigment Black 1, 7, 31 and 32 can be used alone or in combination.

Other examples of the dyes used for color filter are C. I. Solvent Yellow 2, 5, 14, 15, 16, 19, 21, 33, 56, 62, 77, 83, 93, 162, 104, 105, 114, 129, 130, 162, C. I. Disperse Yellow 3, 4, 7, 31, 54, 61, 201, C. I. Direct Yellow 1, 11, 12, 28, C. I. Acid Yellow 1, 3, 11, 17, 23, 38, 40, 42, 76, 98, C. I. Basic Yellow 1, C. I. Solvent Violet 13, 33, 45, 46, C. I. Disperse Violet 22, 24, 26, 28, 31, C. I. Acid Violet 49, C. I. Basic Violet 2, 7, 10, C. I. Solvent Orange 1, 2, 5, 6, 37, 45, 62, 99, C. I. Acid Orange 1, 7, 8, 10, 20, 24, 28, 33, 56, 74, C. I. Direct Orange 1, C. I. Disperse Orange 5, C. I. Direct Brown 6, 58, 95, 101, 173, C. I. Acid Brown 14, C. I. Solvent Black 3, 5, 7, 27, 28, 29, 35, 45 and 46.

In some special cases of manufacturing color filters, complementary colors, yellow, magenta, cyan and optionally green, are used instead of red, green and blue. As yellow for this type of color filters, the abovementioned yellow pigments and dyes can be employed. Examples of the colorants suitable for magenta color are C. I. Pigment Red 122, 144, 146, 169, 177, C. I. Pigment Violet 19 and 23. Examples of cyan color are aluminum phthalocyanine pigments, titanium phthalocyanine pigments, cobalt phthalocyanine pigments, and tin phthalocyanine pigments.

The pigments in the color filter resist composition have preferably a mean particle diameter smaller than the wavelength of visible light (400 nm to 700 nm). Particularly preferred is a mean pigment diameter of <100 nm.

The concentration of the pigment in the total solid component (pigments of various colors and resin) is for example in the range of 5% to 80% by weight, in particular in the range of 20% to 65% by weight.

The concentration of the dye in the total solid component (dyes of various colors and resin) is for example in the range of 0.5% to 95% by weight, in particular in the range of 0.5% to 70% by weight.

If necessary, the pigments may be stabilized in the photosensitive composition by pretreatment of the pigments with a dispersant to improve the dispersion stability of the pigment in the liquid formulation. Suitable additives are described below.

Additives (d) are optionally present in the composition of the invention, such as dispersing agents, surfactant, adhesion promoters, photosensitizer and the like.

It is preferred to apply a surface treatment to the pigments in order to make the pigment easy to disperse and to stabilize the resultant pigment dispersion. The surface treatment reagents are, for example, surfactants, polymeric dispersants, general texture improving agents, pigment derivatives and mixtures thereof. It is especially preferred when the colorant composition according to the invention comprises at least one polymeric dispersant and/or at least pigment derivative.

Suitable surfactants include anionic surfactants such as alkylbenzene- or alkylnaphthalene-sulfonates, alkylsulfosuccinates or naphthalene formaldehyde sulfonates; cationic surfactants including, for example, quaternary salts such as benzyl tributyl ammonium chloride; or nonionic or amphoteric surfactants such as polyoxyethylene surfactants and alkyl- or amidopropyl betaines, respectively.

Illustrative examples of the surfactant include polyoxyethylene alkyl ethers such as polyoxyethylene lauryl ether, polyoxyethylene stearyl ether and polyoxyethylene oleyl ether; polyoxyethylene alkylphenyl ethers such as polyoxyethylene octylphenyl ether and polyoxyethylene nonylphenyl ether; polyethylene glycol diesters such as polyethylene glycol dilaurate and polyethylene glycol distearate; sorbitan fatty acid esters; fatty acid modified polyesters; tertiary amine modified polyurethanes; polyethyleneimines; those available under the trade names of KP (a product of Shin-Etsu Chemical Co., Ltd), Polyflow (a product of KYOEISHA CHEMICAL Co., Ltd), F-Top (a product of Tochem Products Co., Ltd), MEGAFAC (a product of Dainippon Ink & Chemicals, Inc.), Fluorad (a product of Sumitomo 3M Ltd), Asahi Guard and Surflon (products of Asahi Glass Co., Ltd); and the like.

These surfactants may be used alone or in admixture of two or more.

The surfactant is generally used in an amount of 50 parts or less by weight, preferably 0 to 30 parts by weight, based on 100 parts by weight of the colorant composition.

Polymeric dispersants include high molecular weight polymers with pigment affinic groups. Examples are: statistical co-polymers comprised from, for instance, styrene derivatives, (meth)acrylates and (meth)acrylamides, and such statistical co-polymers modified by post modification; block co-polymers and/or comb polymers comprised from, for instance, styrene derivatives, (meth)acrylates and (meth)acrylamides, and such block co-polymers and/or comb polymers modified by post modification; polyethylenimines, which for instance is crafted with polyesters; polyamines, which for instance is crafted with polyesters; and many kinds of (modified) polyurethanes.

Polymeric dispersants may also be employed. Suitable polymeric dispersants are, for example, BYK's DISPERBYK® 101, 115, 130, 140, 160, 161, 162, 163, 164, 166, 168, 169, 170, 171, 180, 182, 2000, 2001, 2009, 2020, 2025, 2050, 2090, 2091, 2095, 2096, 2150, Ciba's Ciba® EFKA® 4008, 4009, 4010, 4015, 4046, 4047, 4050, 4055, 4060, 4080, 4300, 4310, 4330, 4340, 4400, 4401, 4402, 4403, 4406, 4500, 4510, 4520, 4530, 4540, 4550, 4560, Ajinomoto Fine Techno's PB®711, 821, 822, 823, 824, 827, Lubrizol's SOLSPERSE® 1320, 13940, 17000, 20000, 21000, 24000, 26000, 27000, 28000, 31845, 32500, 32550, 32600, 33500, 34750, 36000, 36600, 37500, 39000, 41090, 44000, 53095 and combinations thereof.

It is preferred to use Ciba® EFKA® 4046, 4047, 4060, 4300, 4310, 4330, 4340, DISPERBYK® 161, 162, 163, 164, 165, 166, 168, 169, 170, 2000, 2001, 2020, 2050, 2090, 2091, 2095, 2096, 2105, 2150, PB®711, 821, 822, 823, 824, 827, SOLSPERSE® 24000, 31845, 32500, 32550, 32600, 33500, 34750, 36000, 36600, 37500, 39000, 41090, 44000, 53095 and combinations thereof as dispersant.

Suitable texture improving agents are, for example, fatty acids such as stearic acid or behenic acid, and fatty amines such as laurylamine and stearylamine. In addition, fatty alcohols or ethoxylated fatty alcohols, polyols such as aliphatic 1,2-diols or epoxidized soy bean oil, waxes, resin acids and resin acid salts may be used for this purpose.

Suitable pigment derivatives are, for example, copper phthalocyanine derivatives such as Ciba's Ciba® EFKA® 6745, Lubrizol's SOLSPERSE® 5000, 12000, BYK's SYNERGIST 2100 and azo derivatives such as Ciba® EFKA® 6750, SOLSPERSE® 22000 and SYNERGIST 2105.

The above mentioned dispersants and surfactants for pigments are for example employed in compositions of the present invention which are used as resist formulations, in particular in color filter formulations.

Subject of the invention also is a photopolymerizable composition as described above as further additive comprising a dispersant or a mixture of dispersants as well as a photopolymerizable composition as described above as further additive comprising a pigment or a mixture of pigments.

In the invention, the content of the dispersing agent is preferably from 1 to 80% by mass, more preferably from 5 to 70% by mass, even more preferably from 10 to 60% by mass, based on the mass of the pigment.

Further suitable additives (d) are for example adhesion improving agents. The curable composition of the invention may contain an adhesion improving agent for increasing adhesion to a hard surface, such as of a support. The adhesion improving agent may be a silane coupling agent, a titanium coupling agent or the like.

Photopolymerization can also be accelerated by adding as component (d) further photosensitizers or coinitiators which shift or broaden the spectral sensitivity. These are, in particular, aromatic compounds, for example benzophenone and derivatives thereof, thioxanthone and derivatives thereof, anthraquinone and derivatives thereof, coumarin and phenothiazine and derivatives thereof, and also 3-(aroylmethylene)thiazolines, rhodanine, camphorquinone, but also eosine, rhodamine, erythrosine, xanthene, thioxanthene, acridine, e.g. 9-phenylacridine, 1,7-bis(9-acridinyl)heptane, 1,5-bis(9-acridinyl)pentane, cyanine and merocyanine dyes.

Specific examples of such compounds are

1. Thioxanthones

Thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9Hthioxanthen-9-one 2-ethylhexylether, 1-ethoxycarbonyl-3-(1-methyl-1-morpholinoethyl)-thioxanthone, 2-methyl-6-dimethoxymethyl-thioxanthone, 2-methyl-6-(1,1-dimethoxpenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthon-2-yloxy)N,N,N-trimethyl-1-propanaminium chloride;

2. Benzophenones

benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)benzophenone, 4,4′-bis(diethylamino)benzophenone, 4,4′-bis(methylethylamino)benzophenone, 4,4′-bis(p-isopropylphenoxy)benzophenone, 4-methyl benzophenone, 2,4,6-trimethylbenzophenone, 4-(4-methylthiophenyl)-benzophenone, 3,3′-dimethyl-4-methoxy benzophenone, methyl-2-benzoylbenzoate, 4-(2-hydroxyethylthio)-benzophenone, 4-(4-tolylthio)benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl1,4,7,10,13-pentaoxatridecyl)-benzophenone, 4-benzoyl-N,N-dimethyl-N-[2-(1-oxo-2-propenyl)oxy]ethyl-benzenemethanaminium chloride;

3. Coumarins

Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis[5,7-di(propoxy)coumarin], 3,3′-carbonyl-bis(7-methoxycoumarin), 3,3′-carbonyl-bis(7-diethylamino-coumarin), 3-isobutyloylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyloyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP09-179299-A and JP09-325209-A, for example 7-[{4-chloro-6-(diethylamino)-S-triazine-2-yl}amino]-3-phenylcoumarin;

4. 3-(aroylmethylene)-thiazolines

3-methyl-2-benzoylmethylene-□-naphthothiazoline, 3-methyl-2-benzoylmethylene-benzothiazoline, 3-ethyl-2-propionylmethylene-□-naphthothiazoline;

5. Rhodanines

4-dimethylaminobenzalrhodanine, 4-diethylaminobenzalrhodanine, 3-ethyl-5-(3-octyl-2-benzothiazolinylidene)-rhodanine, the rhodanine derivatives, formulae [1], [2], [7], disclosed in JP08-305019A;

6. Other Compounds

Acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, phenanthrenequinone, 9-fluorenone, dibenzosuberone, curcumin, xanthone, thiomichler's ketone, α-(4-dimethylaminobenzylidene) ketones, e.g. 2,5-bis(4-diethylaminobenzylidene)cyclopentanone, 2-(4-dimethylamino-benzylidene)-indan-1-one, 3-(4-dimethylamino-phenyl)-1-indan-5-yl-propenone, 3-phenylthiophthalimide, N-methyl-3,5-di(ethylthio)-phthalimide, N-methyl-3,5-di(ethylthio)phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propylenegylcol)-4-(dimethylamino) benzoate.

A photosensitizer may be selected from the group consisting of benzophenone and its derivatives, thioxanthone and its derivatives, anthraquinone and its derivatives, or coumarin and its derivatives.

To accelerate the photopolymerization, it is possible to add amines, for example triethanolamine, N-methyldiethanolamine, ethyl-p-dimethylaminobenzoate, 2-(dimethylamino)ethyl benzoate, 2-ethylhexyl-p-dimethylaminobenzoate, octyl-para-N,N-dimethylaminobenzoate, N-(2-hydroxyethyl)-N-methyl-para-toluidine or Michler's ketone. The action of the amines can be intensified by the addition of aromatic ketones of the benzophenone type. Examples of amines which can be used as oxygen scavengers are substituted N,N-dialkylanilines, as are described in EP339841. Other accelerators, coinitiators and autoxidizers are thiols, thioethers, disulfides, phosphonium salts, phosphine oxides or phosphines, as described, for example, in EP438123, in GB2180358 and in JP KokaiHei 6-68309.

To accelerate the polymerization thermally, it is possible to add thermal curing promoter, e.g., oxime sulfonates, as are described, for example, in WO2012/101245, hydroxylamine esters, as are described, for example, in WO2012/108835, in WO2001090113, in WO03029332 and in WO04081100, peroxides, such as organic peroxides or hydroperoxides, as are described, for example, in JP2003015288 and in JP10010718, and azo compounds, are described, for example, in JP2003015288.

The choice of additive(s) is made depending on the field of application and on properties required for this field. The additives described above are customary in the art and accordingly are added in amounts which are usual in the respective application.

Thermal inhibitors as optional further additives (d) are intended to prevent premature polymerization, examples being hydroquinone, hydroquinone derivatives, p-methoxyphenol, β-naphthol or sterically hindered phenols, such as 2,6-di-t-butyl-p-cresol. In order to increase the stability on storage in the dark it is possible, for example, to use copper compounds, such as copper naphthenate, stearate or octoate, phosphorus compounds, for example triphenylphosphine, tributylphosphine, triethylphosphine, triphenyl phosphate or tribenzyl phosphate, quaternary ammonium compounds, for example tetramethylammonium chloride or trimethylbenzylammonium chloride, or hydroxylamine derivatives, for example N-diethylhydroxylamine. To exclude atmospheric oxygen during the polymerization it is possible to add paraffin or similar wax-like substances which, being of inadequate solubility in the polymer, migrate to the surface in the beginning of polymerization and form a transparent surface layer which prevents the ingress of air. It is also possible to apply an oxygen-impermeable layer. Light stabilizers which can be added in a small quantity are UV absorbers, for example those of the hydroxphenylbenzotriazole, hydroxyphenyl-benzophenone, oxalamide or hydroxyphenyl-s-triazine type. These compounds can be used individually or in mixtures, with or without sterically hindered amines (HALS).

Thermal inhibitors can be used to prevent deterioration of color properties like transparency of the compositions, preferably phenol derivatives or sterically hindered phenols as are described, for example, in U.S. Pat. No. 4,994,628, in JP6128195, JP7206771, and in WO0198249.

Further, the latent thermal inhibitors are also used as antioxidant to prevent premature polymerization or discoloration. The latent thermal inhibitors are a compound having a protective group capable of being desorbed by heating, which is a compound that develops an antioxidant function by desorption of the protecting group. As preferable latent thermal inhibitors, the compounds which synthesized with a combination of phenol derivatives and acid anhydride, Boc reagent such as di-t-butyl dicarbonate, acid chloride, alkyl halide derivatives, allyl ether derivatives or cyclochloride derivatives are known, for example, in WO14021023, in WO17043353, in WO2016056290, in JP2017008219, in JP2017066370, in JP201513937 in WO2018062105

The compositions according to the invention may also comprise one or more solvents. Examples of suitable solvents are ketones, ethers and esters, such as methyl ethyl ketone, isobutyl methyl ketone, cyclopentanone, cyclohexanone, N-methylpyrrolidone, dioxane, tetrahydrofuran, ethylene glycol monomethyl ether, ethylene glycol monoethyl ether, ethylene glycol monopropyl ether, ethylene glycol monobutyl ether, ethylene glycol dimethyl ether, ethylene glycol diethyl ether, ethylene glycol dipropyl ether, propylene glycol monomethyl ether, propylene glycol monoethyl ether, propylene glycol monopropyl ether, propylene glycol monobutyl ether, propylene glycol dimethyl ether, propylene glycol diethyl ether, propylene glycol dipropyl ether, diethylene glycol monomethyl ether, diethylene glycol monoethyl ether, diethylene glycol dimethyl ether, diethylene glycol diethyl ether, ethylene glycol monomethyl ether acetate, ethylene glycol monoethyl ether acetate, ethylene glycol monopropyl ether acetate, ethylene glycol monobutyl ether acetate, propylene glycol monomethyl ether acetate, propylene glycol monoethyl ether acetate, propylene glycol monopropyl ether acetate, 2-methoxybutyl acetate, 3-methoxybutyl acetate, 4-methoxybutyl acetate, 2-methyl-3-methoxybutyl acetate, 3-methyl-3-methoxybutyl acetate, 3-ethyl-3-methoxybutyl acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, ethyl acetate, n-butyl acetate, ethyl propionate, propyl propionate, butyl propionate, ethyl 3-ethoxypropionate, methyl 3-methoxypropionate, 2-heptanone, 2-pentanone, and ethyl lactate.

The compositions according to this invention can comprise additionally a compound which generates radicals thermally, examples being organic peroxide, azo derivatives, benzoin derivatives, benzoin ether derivatives, acetophenone derivatives, hydroxylamine esters, oxime derivatives and hydrogen peroxides.

Examples of commercially available peroxide are dilauroyl, 1,1,3,3-tetramethylbutyl peroxy-2-ethylhexanoate, t-butyl peroxy-2-ethylhexanoate, 1,1-di(t-butylperoxy)-2-methylcyclohexane, 1,1-di(t-hexylperoxy)-3,3,5-trimethylcyclohexane, t-butylperoxymaleic acid, t-butyl peroxylaurate, t-butyl peroxy 2-ethylhexyl monocarbonate, t-hexyl peroxybenzoate, t-butyl peroxyacetate, t-butyl peroxybenzoate, Dicymyl peroxide, di-t-butyl peroxide, 2,5-dimethyl-2,5-di(t-butylperoxy)hexyne-3, 1,1,3,3-tetramethylbutyl hydroperoxide, cumene hydroperoxide, 2,3-dimethyl-2,3-diphenylbutane, (NOF Co., LTD.), Kayamek A, Kayamek M, Kayamek R, Kayamek L, Kayamec LH, (Kayaku Akzo Co., LTD.) for example described in JP2013-014675.

Examples of commercially available azo derivatives are 2,2′-azobis(isobutyronitrile), 2,2′-azobis(2-methylbutyronitrile), 1,1′-azobis(cyclohexane-1-1-carbonitrile), (2,4-dimethylvaleronitrile), 1-[(1-cyano-1-methylethyl)azo]formamide, 2,2’-azobis(2-amidinopropane) dihydrochloride, 2,2′-azobis[N-(2-propenyl)-2-methylpropionamide], 2,2′-azobis(N-butyl-2-methylpropionamide), 2,2′-azobis[2-(2-imidazolin-2-yl)propane], 2,2′-azobis[2-methyl-N-(2-hydroxyethyl)propionamide], Dimethyl 2,2′-azobis(isobutyrate) (Wako pure Chemical Industries., LTD.)

Other thermal radical initiators are hydroxylamine derivatives, as described, for example, oxime derivatives, in WO10/108835 and oxime derivatives such as oxime sulfonate, as described, for example, in WO12/101245 and WO16/030790.

The compositions according to this invention can comprise additionally a crosslinking agent which is activated by an acid or a base, for example as described in JP 10 221843-A, and a compound which generates acid or base thermally or by actinic radiation and which activates a crosslinking reaction. Use is made, in addition to the free-radical hardeners, of cationic photo or thermal initiators such as sulfonium-, phosphonium- or iodonium salts, for example IRGACURE®250, San-Aid SI series, SI-60L, SI-80L, SI-100L, SI-110L, SI-145, SI-150, SI-160, SI-180L produced by Sanshin Chemical, cyclopentadienyl-arene-iron(II) complex salts, for example (η⁶-iso-propylbenzene)(η⁵-cyclopentadienyl)iron(II) hexafluorophosphate, as well as oxime sulfonic acid esters, for example described in EP 780729. Also pyridinium and i-quinolinium salts as described e.g. in EP 497531 and EP 441232 may be used in combination with the new photoinitiators. Examples of bases are imidazole and its derivatives for example Curezole OR series and CN series provided by Shikoku Chemicals.

The crosslinking agents which can be activated by acid or base include compounds having epoxy or oxetane groups. There may be used a solid or liquid known epoxy or oxetane compound and said compound is used depending on required characteristics. A preferred epoxy resin is a bisphenol S type epoxy resin such as BPS-200 produced by Nippon Kayaku Co., Ltd., EPX-30 produced by ACR Co., Epiculon EXA-1514 produced by Dainippon Ink & Chemicals Inc., etc.; a bisphenol A type epoxy resin such as Epiculon N-3050, N-7050, N-9050 produced by Dainippon Ink & Chemicals Inc., XAC-5005, GT-7004, 6484T, 6099; a bisphenol F type epoxy resin such as YDF-2004, YDF2007 produced by NSCC Epoxy Manufacturing Co., Ltd., etc.; a bisphenol fluorene type epoxy resin such as OGSOL PG, PG-100, EG, EG-210 produced by Osaka Gas Chemicals; a diglycidyl phthalate resin such as Blemmer DGT produced by Nippon Oil and Fats Co., Ltd., etc.; a heterocyclic epoxy resin such as TEPIC produced by Nissan Chemical Industries, Ltd., Araldite PT810 produced by Ciba Specialty Chemicals Inc., etc.; a bixylenol type epoxy resin such as YX-4000 produced by Yuka Shell Co., etc.; a biphenol type epoxy resin such as YL-6056 produced by Yuka Shell Co., etc.; a tetraglycidyl xylenoylethane resin such as ZX-1063 produced by NSCC Epoxy Manufacturing Co., Ltd., etc.; a novolak type epoxy resin such as EPPN-201, EOCN-103, EOCN-1020, EOCN-1025 and BRRN produced by Nippon Kayaku Co., Ltd., ECN-278, ECN-292 and ECN-299 produced by Asahi Chemical Industry Co., Ltd., GY-1180, ECN-1273 and ECN-1299 produced by BASF Japan Ltd., YDCN-220L, YDCN-220HH, YDCN-702, YDCN-704, YDPN-601 and YDPN-602 produced by NSCC Epoxy Manufacturing Co., Ltd., Epiculon-673, N-680, N-695, N-770 and N-775 produced by Dainippon Ink & Chemicals Inc., etc.; a novolak type epoxy resin of bisphenol A such as EPX-8001, EPX-8002, EPPX-8060 and EPPX-8061 produced by Asahi Chemical Industry Co., Ltd., Epiculon N-880 produced by Dainippon Ink & Chemicals Inc., etc.; a chelate type epoxy resin such as EPX-49-69 and EPX-49-30 produced by Asahi Denka Kogyo K.K., etc.; a glyoxal type epoxy resin such as YDG-414 produced by NSCC Epoxy Manufacturing Co., Ltd., etc.; an amino group-containing epoxy resin such as YH-1402 and ST-110 produced by NSCC Epoxy Manufacturing Co., Ltd., YL-931 and YL-933 produced by Yuka Shell Co., etc.; a rubber-modified epoxy resin such as Epiculon TSR-601 produced by Dainippon Ink & Chemicals Inc., EPX-84-2 and EPX-4061 produced by Asahi Denka Kogyo K.K., etc.; a dicyclopentadiene phenolic type epoxy resin such as DCE-400 produced by Sanyo-Kokusaku Pulp Co., Ltd., etc.; a silicone-modified epoxy resin such as X-1359 produced by Asahi Denka Kogyo K.K., etc.; an e-caprolactone-modified epoxy resin such as Plaque G-402 and G-710 produced by Daicel Co., etc. and others. Further, partially esterified compounds of these epoxy compounds (e.g. esterified by (meth)acrylates) can be used in combination. Examples of oxetane compounds are 3-ethyl-3-hydroxmethyloxetane (oxetane alcohol), 2-ethylhexyloxetane, xylene bisoxetane, 3-ethyl-3[[(3-ethyloxetane-3-yl)methoxy]methyl]oxetane (Aron Oxetane series) provided by Toagosei Co., Ltd.

The photopolymerizable compositions can be used for various purposes, for example as printing ink, e.g. screen printing inks, inks for offset- or flexo printing, as a clear finish, as a white or colored finish, for example for wood or metal, as powder coating, as a coating material, inter alia for paper, wood, metal or plastic, as a daylight-curable coating for the marking of buildings and roadmarking, for photographic reproduction techniques, for holographic recording materials, for image recording techniques or to produce printing plates which can be developed with organic solvents or with aqueous alkalis, for producing masks for screen printing, as dental filling compositions, as adhesives, as pressure-sensitive adhesives, as laminating resins, as etch resists, solder resists, electroplating resists, or permanent resists, both liquid and dry films, as photostructurable dielectric, for printed circuit boards and electronic circuits, as resists to manufacture color filters for a variety of display applications or to generate structures in the manufacturing process of plasma-display panels and electroluminescence displays, (as for example described in U.S. Pat. No. 5,853,446, EP863534, JP 09-244230-A, JP10-62980-A, JP08-171863-A, U.S. Pat. No. 5,840,465, EP855731, JP05-271576-A, JP 05-67405-A) for the production of holographic data storage (HDS) material, for the production of optical switches, optical lattices (interference lattice), light circuits, for producing three-dimensional articles by mass curing (UV curing in transparent moulds) or by the stereo-lithography technique, as is described, for example, in U.S. Pat. No. 4,575,330, to produce composite materials (for example styrenic polyesters, which may, if desired, contain glass fibres and/or other fibres and other auxiliaries) and other thick-layered compositions, for coating or sealing electronic components and integrated circuits, or as coatings for optical fibres, or for producing optical lenses, e.g. contact lenses or Fresnel lenses. The compositions according to the invention are further suitable for the production of medical equipment, auxiliaries or implants. Further, the compositions according to the invention are suitable for the preparation of gels with thermotropic properties, as for example described in DE19700064 and EP678534.

The novel photoinitiators may additionally be employed as initiators for emulsion polymerizations, pearl polymerizations or suspension polymerizations, as polymerization initiators for fixing ordered states of liquid-crystalline monomers and oligomers, or as initiators for fixing dyes on organic materials.

In coating materials, use is frequently made of mixtures of a prepolymer with polyunsaturated monomers, which may additionally include a monounsaturated monomer as well. It is the prepolymer here which primarily dictates the properties of the coating film, and by varying it the skilled worker is able to influence the properties of the cured film. The polyunsaturated monomer functions as a crosslinking agent which renders the film insoluble. The monounsaturated monomer functions as a reactive diluent, which is used to reduce the viscosity without the need to employ a solvent.

Unsaturated polyester resins are usually used in two-component systems together with a monounsaturated monomer, preferably with styrene. For photoresists, specific one-component systems are often used, for example polymaleimides, polychalcones or polyimides, as described in DE 2308830.

The novel photoinitiators can also be used for the polymerization of radiation-curable powder coatings. The powder coatings can be based on solid resins and monomers containing reactive double bonds, for example maleates, vinyl ethers, acrylates, acrylamides and mixtures thereof.

The novel photocurable compositions are suitable, for example, as coating materials for substrates of all kinds, for example wood, textiles, paper, ceramics, glass, plastics such as polyesters, polyethylene terephthalate, polyolefins or cellulose acetate, especially in the form of films, and also metals such as Al, Cu, Ni, Fe, Zn, Mg or Co and GaAs, Si or SiO₂ to which it is intended to apply a protective layer or, by means of imagewise exposure, to generate an image.

The novel radiation-sensitive compositions further find application as negative resists, having a very high sensitivity to light and being able to be developed in an aqueous alkaline medium without swelling. They are suitable for the production of printing forms for relief printing, planographic printing, photogravure or of screen printing forms, for the production of relief copies, for example for the production of texts in braille, for the production of stamps, for use in chemical milling or as a microresist in the production of integrated circuits. The compositions further may be used as photopatternable dielectric layer or coating, encapsulating material and isolating coating in the production of computer chips, printed boards and other electric or electronic components. The possible layer supports, and the processing conditions of the coating substrates, are just as varied.

The novel composition also relates to a photosensitive thermosetting resin composition and a method of forming a solder resist pattern by the use thereof, and more particularly relates to a novel photosensitive thermosetting resin composition useful as materials for the production of printed circuit boards, the precision fabrication of metallic articles, the etching of glass and stone articles, the relief of plastic articles, and the preparation of printing plates and particularly useful as a solder resist for printed circuit boards and to a method of forming a solder resist pattern by the steps of exposing a layer of the resin composition selectively to an actinic ray through a photomask having a pattern and developing the unexposed part of the layer.

The solder resist is a substance which is used during the soldering of a given part to a printed circuit board for the purpose of preventing molten solder from adhering to irrelevant portions and protecting circuits. It is, therefore, required to possess such properties as high adhesion, insulation resistance, resistance to soldering temperature, resistance to solvents, resistance to alkalis, resistance to acids, and resistance to plating.

Because the photocurable compositions according to the invention have a good thermal stability and are sufficiently resistant to inhibition by oxygen, they are particularly suitable for the production of color filters or color mosaic systems, such as described, for example, in EP320264.

Color filters usually are employed in the manufacturing of flat panel displays such as LCD's, PDP(plasma panel display), EL (electroluminessence) display, and projection systems, image sensors, CCD (charge coupled device), and CMOS (complementary metal oxide semiconductor) sensors for scanner, digital camera and video camera. The color filters usually are prepared by forming red, green and blue pixels and a black matrix on a glass substrate. In these processes photocurable compositions according to the invention can be employed. A particularly preferred method of use comprises adding of the coloring matters, dyes and pigments of red, green and blue colors to the light-sensitive resin composition of the present invention, coating of the substrate with the composition, drying of the coating with a short heat treatment, patternwise exposure of the coating to actinic radiation and subsequent development of the pattern in an aqueous alkaline developer solution and optionally a heat treatment. Thus, by subsequently applying a red, green and blue pigmented coating, in any desired order, on top of each other with this process a color filter layer with red, green and blue color pixels can be produced.

The development is carried out by washing out the areas which were not polymerized with a suitable alkali developing solution. This process is repeated to form the image having plural colors.

In the light-sensitive resin composition of the present invention, with a process in which at least one or more picture elements are formed on a transparent substrate and then an exposure is given from a side of the transparent substrate, on which the above picture elements are not formed, the above picture elements can be utilized as a light-shielding mask. In this case, for example, in the case where an overall exposure is given, a position adjustment of a mask gets unnecessary and a concern on a position slippage thereof is removed. And, it is possible to cure all of the part on which the above picture elements are not formed. Further, in this case, it is possible as well to develop and remove a part of the portion on which the above picture elements are not formed by using partially a light-shielding mask.

Since in either case, no gap is formed between the picture elements which are formed formerly and those which are formed later, the composition of the present invention is suitable for, for example, a forming material for a color filter. To be concrete, the coloring matters, dyes and pigments of red, green and blue colors are added to the light-sensitive resin composition of the present invention, and the processes for forming an image are repeated to form the picture elements of red, green and blue colors. Then, the light-sensitive resin composition to which, for example, the black coloring materials, dyes and pigments are added is provided on an overall face. An overall exposure (or a partial exposure via a light-shielding mask) can be provided thereon to form the picture elements of a black color all over the spaces (or all but a partial region of the light-shielding mask) between the picture elements of red, green and blue colors. In addition to a process in which the light-sensitive resin composition is coated on a substrate and dried, the light-sensitive resin composition of the present invention can be used as well for a layer transfer material. That is, the light-sensitive resin composition is layer-wise provided directly on a temporary support, preferably on a polyethylene terephthalate film, or on a polyethylene terephthalate film on which an oxygen-shielding layer and a peeling layer or the peeling layer and the oxygen-shielding layer are provided. Usually, a removable cover sheet made of a synthetic resin is laminated thereon for a protection in handling. Further, there can be applied as well a layer structure in which an alkali soluble thermoplastic resin layer and an intermediate layer are provided on a temporary support and further a light-sensitive resin composition layer is provided thereon (JP 5-173320-A).

The above cover sheet is removed in use and the light-sensitive resin composition layer is laminated on a permanent support. Subsequently, peeling is carried out between those layer and a temporary support when an oxygen-shielding layer and a peeling layer are provided, between the peeling layer and the oxygen-shielding layer when the peeling layer and the oxygen-shielding layer are provided, and between the temporary support and the light-sensitive resin composition layer when either the peeling layer or the oxygen-shielding layer is not provided, and the temporary support is removed.

A metal support, glass, ceramics, and a synthetic resin film can be used as a support for a color filter. Glass and a synthetic resin film which is transparent and have an excellent dimension stability is particularly preferred. The thickness of the light-sensitive resin composition layer is usually 0.1 to 50 micrometers, in particular 0.5 to 5 micrometers.

The developer solution can be used in all forms known to the person skilled in the art, for example in form of a bath solution, puddle, or a spraying solution. In order to remove the non-cured portion of the light-sensitive resin composition layer, there can be combined the methods such as rubbing with a rotary brush and rubbing with a wet sponge. Usually, the temperature of the developing solution is preferably at and around room temperature to 40° C. The developing time is changeable according to the specific kind of the light-sensitive resin composition, the alkalinity and temperature of the developing solution, and the kind and concentration of the organic solvent in the case where it is added. Usually, it is 10 seconds to 2 minutes. It is possible to put a rinsing step after the development processing.

A final heat treatment is preferably carried out after the development processing. Accordingly, a support having a layer which is photopolymerized by exposing (hereinafter referred to as a photocured layer) is heated in an electric furnace and a drier, or the photocured layer is irradiated with an infrared lamp or heated on a hot plate. The heating temperature and time depend on the composition used and the thickness of the formed layer. In general, heating is preferably applied at about 120° C. to about 250° C., for about 5 to about 60 minutes.

The compositions according to this invention can also comprise latent pigments which are transformed into finely dispersed pigments during the heat treatment of the latent pigment containing photosensitive pattern or coating. The heat treatment can be performed after exposure or after development of the latent pigment-containing photoimageable layer. Such latent pigments are soluble pigment precursors which can be transformed into insoluble pigments by means of chemical, thermal, photolytic or radiation induced methods as described, for example, in U.S. Pat. No. 5,879,855. This transformation of such latent pigments can be enhanced by adding a compound which generates acid at actinic exposure or by adding an acidic compound to the composition. Therefore, a color filter resist can also be prepared, which comprises a latent pigment in a composition according to this invention.

Examples of color filters, especially with respect to the above described combinations of pigments and ionic impurity scavenger are given in EP320264. It is understood, that the photoinitiators according to the present invention, i.e. the compounds of the formula I in the color filter formulations described in EP 320264 can replace the triazine initiator compounds. Suitable components for a color filter compositions are described above in more detail.

Examples for color filter resists, the composition of such resists and the processing conditions are given by T. Kudo et al., Jpn. J. Appl. Phys. Vol. 37 (1998) 3594; T. Kudo et al., J. Photopolym. Sci. Technol. Vol 9 (1996) 109; K. Kobayashi, Solid State Technol. November 1992, p. S15-S18; U.S. Pat. Nos. 5,368,976; 5,800,952; 5,882,843; 5,879,855; 5,866,298; 5,863,678; JP 06-230212A; EP320264; JP 09-269410A; JP 10-221843A; JP 01-090516A; JP 10-171 119A, U.S. Pat. Nos. 5,821,016, 5,847,015, 5,882,843, 5,719,008, EP881541, or EP902327.

The photoinitiators of the present invention can be used in color filter resists, for example, such as those given as examples above, or can partially or fully replace the known photoinitiators in such resists. It is understood by a person skilled in the art that the use of the new photoinitiators of the present invention is not limited to the specific binder resins, crosslinkers and formulations of the color filter resist examples given hereinbefore but can be used in conjunction with any radically polymerizable component in combination with a dye or color pigment or latent pigment to form a photosensitive color filter ink or color filter resist.

Accordingly, subject of the invention also is a color filter prepared by providing red, green and blue (RGB) colour elements and, optionally a black matrix, all comprising a photosensitive resin and a pigment on a transparent substrate and providing a transparent electrode either on the surface of the substrate or on the surface of the color filter layer, wherein said photosensitive resin comprises a polyfunctional acrylate monomer, an organic polymer binder and a photopolymerization initiator of formula I as described above. The monomer and binder components, as well as suitable pigments are as described above. In the manufacture of color filters the transparent electrode layer can either be applied on the surface of the transparent substrate or can be provided on the surface of the red, green and blue picture elements and the black matrix. The transparent substrate is for example a glass substrate which can additionally have an electrode layer on its surface.

It is preferred to apply a black matrix between the color areas of different color in order to improve the contrast of a color filter. The photosensitive compositions of the present invention, as already stated above, are also suitable for the preparation of the black matrix of color filters.

Said black matrix composition for example comprises

a photoinitiator compound of the formula I of the present invention,

an organic binder, in particular an organic binder, which is an epoxy acrylate resin having a carboxyl group,

a black coloring material,

a polymer dispersant, in particular a polymer dispersant containing a basic functional group.

The person skilled in the art is familiar with such formulations. Examples of suitable black matrix compositions and the components (other than the photoinitiator) as described above are given in JP Patent No. 3754065, the disclosure of which hereby is incorporated by reference.

Instead of forming a black matrix using a photosensitive composition and patterning the black photosensitive composition photolithographically by patternwise exposure (i.e. through a suitable mask) to form the black pattern separating the red green and blue coloured areas on the transparent substrate it is alternatively possible to use an inorganic black matrix. Such inorganic black matrix can be formed from deposited (i.e. sputtered) metal (i.e. chromium) film on the transparent substrate by a suitable imaging process, for example utilizing photolithographic patterning by means of an etch resist, etching the inorganic layer in the areas not protected by the etch resist and then removing the remaining etch resist.

There are different methods known how and at which step in the color filter manufacturing process the black matrix can be applied. It can either be applied directly on the transparent substrate prior to formation of the red, green and blue (RGB) colour filter as already mentioned above, or it can be applied after the RGB colour filter is formed on the substrate.

In a different embodiment of a color filter for a liquid crystal display, according to US626796, the black matrix can also be applied on the substrate opposite to the RGB color filter element-carrying substrate, which is separated from the former by a liquid crystal layer. If the transparent electrode layer is deposited after applying the RGB color filter elements and—optionally—the black matrix, an additional overcoat film as a protective layer can be applied on the color filter layer prior to deposition of the electrode layer, for example, as described in U.S. Pat. No. 5,650,263.

To form an overcoat layer of a color filter, photosensitive resin or thermosetting resin compositions are employed. The photosensitive composition of the present invention can also be used to form such overcoat layers, because a cured film of the composition is excellent in flatness, hardness, chemical and thermal resistance, transparency especially in a visible region, adhesion to a substrate, and suitability for forming a transparent conductive film, e.g., an ITO film, thereon. In the production of a protective layer, there has been a demand that unnecessary parts of the protective layer, for example on scribing lines for cutting the substrate and on bonding pads of solid image sensors should be removed from the substrate as described in JP57-42009A, JP1-130103A and JP1-134306A. In this regard, it is difficult to selectively form a protective layer with good precision using the above-mentioned thermosetting resins. The photosensitive composition, however, allows to easily remove the unnecessary parts of the protective layer by photolithography. It is obvious to those skilled in the art, that the photosensitive compositions of the present invention can be used for generating red, green and blue color pixels and a black matrix, for the manufacture of a color filter, regardless of the above described differences in processing, regardless, of additional layers which can be applied and regardless of differences in the design of the color filter. The use of a composition according to the present invention to form colored elements shall not be regarded as limited by different designs and manufacturing processes of such color filters.

The photo-sensitive composition of the present invention can suitably be used for forming a color filter but will not be limited to this application. It is useful as well for a recording material, a resist material, a protective layer, a dielectric layer, in display applications and display elements, a paint, and a printing ink.

The photosensitive compositions according to the invention are also suitable for manufacturing interlayer insulating layers or dielectric layers in a liquid crystal display, and more particularly in a reflection type liquid crystal display including an active matrix type display having a thin film transistor (TFT) as a switching device, and a passive matrix type without a switching device.

In recent years, liquid crystal displays have, for example, been widely used for pocket-type TV sets and terminal devices for communication by virtue of its small thickness and light weight. A reflection type liquid crystal display without necessity of using a back light is in particular in demand because it is ultra-thin and light-weight, and it can significantly reduce power consumption. However, even if a back light is removed out of a presently available transmission type color liquid crystal display and a light reflection plate is added to a lower surface of the display, it would cause a problem in that the efficiency of utilizing lights is low, and it is not possible to have practical brightness. As a solution to this problem, there have been suggested various reflection type liquid crystal displays for enhancing an efficiency of utilizing lights. For instance, a certain reflection type liquid crystal display is designed to include a pixel electrode having reflection function.

The reflection type liquid crystal display includes an insulating substrate and an opposing substrate spaced away from the insulating substrate. A space between the substrates is filled with liquid crystals. A gate electrode is formed on the insulating substrate, and both the gate electrode and the insulating substrate are covered with a gate insulating film. A semiconductor layer is then formed on the gate insulating film above the gate electrode. A source electrode and a drain electrode are also formed on the gate insulating film in contact with the semiconductor layer. The source electrode, the drain electrode, the semiconductor layer, and the gate electrode cooperate with one another to thereby constitute a bottom gate type TFT as a switching device.

An interlayer insulating film is formed covering the source electrode, the drain electrode, the semiconductor layer, and the gate insulating film therewith. A contact hole is formed throughout the interlayer insulating film on the drain electrode. A pixel electrode made of aluminum is formed on both the interlayer insulating film and an inner sidewall of the contact hole. The drain electrode of the TFT is eventually in contact with the pixel electrode through the interlayer insulating film. The interlayer insulating layer is generally designed to have a roughened surface by which the pixel electrode acts as a reflection plate which diffuses lights to get a wider angle for viewing (angle of visibility).

The reflection type liquid crystal display remarkably enhances an efficiency of using lights by virtue that the pixel electrode acts as a light reflection plate.

In the above-mentioned reflection type liquid crystal display, the interlayer insulating film is designed to have projections and recesses by photolithography. To form and control a fine shape of the projections and recesses in micrometer order for surface roughness and to form contact holes, photolithography methods using positive and negative photoresists are used. For these resists the compositions according to the invention are especially suitable.

The photosensitive compositions according to the invention can further be used for manufacturing spacers, which control a cell gap of the liquid crystal part in liquid crystal display panels. A transparent column spacer has been widely used in the LCD technology, but the transparent spacer disturbs polarized light reducing the contrast ratio. One of a possible solution is to mix with a black colorant not to scatter but to absorb the polarized light, i.e. a black column spacer. Black column spacer is also used in the LCD technology. In case of black column spacer, one or more further black colorants or mixture of other color colorants described above colorant is used.

Since the properties of light transmitted or reflected through the liquid crystal layer in a liquid crystal display are dependent on the cell gap, the thickness accuracy and uniformity over the pixel array are critical parameters for the performance of the liquid crystal display unit. In a liquid crystal cell, the spacing between the substrates in the cell is maintained constant by sparsely distributing glass or polymer spheres about several micrometers in diameter as spacers between the substrates. The spacers are thus held between the substrates to maintain the distance between the substrates at a constant value. The distance is determined by the diameter of the spacers. The spacers assure the minimum spacing between the substrates; i.e., they prevent a decrease in distance between the substrates. However, they cannot prevent the substrates from being separated apart from each other, i.e. the increase in distance between the substrates. Additionally, this method of using spacer beads has problems of the uniformity in the diameter of spacer beads and difficulty in the even dispersion of spacer beads on the panel, as well as non-uniform orientation and decrease in brightness and/or optical aperture depending on the location of spacers on pixel array region. Liquid crystal displays having a large image display area have recently been attracting much attention. However, the increase in the area of a liquid crystal cell generally produces the distortion of the substrates constituting the cell. The layer structure of the liquid crystal tends to be destroyed due to the deformation of the substrate. Thus, even when spacers are used for maintaining the spacing between the substrates constant, a liquid crystal display having a large image display area is unfeasible because the display experiences disturbances. Instead of the above spacer sphere dispersion method, a method of forming columns in the cell gap as spacers has been proposed. In this method, columns of a resin are formed as spacers in the region between the pixel array region and the counter electrode to form a prescribed cell gap. Photosensitive materials having adhesive properties with photolithography are commonly used, for instance, in the manufacturing process of color filters. This method is advantageous compared with the conventional method using spacer beads in the points that location, number and height of the spacers may be controlled freely. In recent years, as the spread of the touch panel type liquid crystal displays such as mobile audio players and handheld game platforms, the mechanical stress to liquid crystal panel tends to grow. The demand for spacer that controls the cell gap to raise mechanical strength becomes strong thus the multi-spacer method is used. According to the multi-spacer method, when cell gap narrows by pressure from the outside, adding to main-spacer that controls the cell gap normally lower sub-spacer supports the cell gap against external stress. The multi-spacer can follow the contraction of liquid crystal at low temperature conditions by main-spacer and prevent to generate bubbles inside the liquid crystal.

The multi-spacer which contains main-spacer and sub-spacer is formed in the same step using, for example, a halftone mask as described in JPA-201 1065133. The photosensitive compositions according to the invention are eligible for manufacturing process using halftone mask.

In a color liquid crystal display panel, such spacers are formed in the nonimaging area under black matrix of color filter elements. Therefore, the spacers formed using photosensitive compositions do not decrease brightness and optical aperture. Photosensitive compositions for producing protective layer with spacers for color filters are disclosed in JP 2000-81701A and dry film type photoresists for spacer materials are also disclosed in JP 11-174459A and JP 11-174464A. As described in the documents, the photosensitive compositions, liquid and dry film photoresists, are comprising at least an alkaline or acid soluble binder polymer, a radically polymerizable monomer, and a radical initiator. In some cases, thermally crosslinkable components such as epoxide and carboxylic acid may additionally be included.

The steps to form spacers using a photosensitive composition are as follows:

a photosensitive composition is applied to the substrate, for instance a color filter panel and after the substrate is prebaked, it is exposed to light through a mask. Then, the substrate is developed with a developer and patterned to form the desired spacers. When the composition contains some thermosetting components, usually a postbaking is carried out to thermally cure the composition.

The photocurable compositions according to the invention are suitable for producing spacers for liquid crystal displays (as described above) because of their high sensitivity.

The photosensitive compositions according to the invention are also suitable for manufacturing microlens arrays used in liquid crystal display panels, image sensors and the like.

Microlenses are microscopic passive optical components that fit on active optoelectronic devices such as detectors, displays, and light emitting devices (light-emitting diodes, transversal and vertical cavity lasers) to improve their optical input or output quality. The areas of applications are wide and cover areas such as telecommunications, information technology, audio-visual services, solar cells, detectors, solid-state light sources, and optical interconnects.

Present optical systems use a variety of techniques to obtain efficient coupling between microlenses and microoptical devices.

The microlens arrays are used for condensing illuminating light on the picture element regions of a nonluminescent display device, such as a liquid crystal display devices, to increase the brightness of the display, for condensing incident light or as a means for forming an image on the photoelectric conversion regions of a line image sensor used for example in facsimiles and the like to improve the sensitivity of these devices, and for forming an image to be printed on a photosensitive means used in liquid crystal printers or light emitting diode (LED) printers.

The most common application is their use to improve the efficiency of photodetector arrays of a solid-state image sensing device such as a charge coupled device (CCD). In a detector array, the collection of as much light as possible in each detector element or pixel is wanted. If a microlens is put on top of each pixel, the lens collects incoming light and focuses it onto an active area that is smaller than the size of the lens.

According to the prior art, microlens arrays can be produced by a variety of methods; for each of them compositions according to the present invention may be employed.

(1) A method for obtaining convex lenses wherein a pattern of the lenses in a planar configuration is drawn on a thermoplastic resin by a conventional photolithographic technique or the like, and then the thermoplastic resin is heated to a temperature above the softening point of the resin to have flowability, thereby causing a sag in the pattern edge (so called “reflowing”) (see, e.g., JP 60-38989A, JP 60-165623A, JP 61-67003A, and JP 2000-39503A). In this method, when the thermoplastic resin used is photosensitive, a pattern of the lenses can be obtained by exposure of this resin to light.

(2) A method for forming a plastic or glass material by the use of a mold or a stamper. As lens material, a photocurable resin and a thermosetting resin can be used in this method (see, e.g., WO99/38035).

(3) A method for forming convex lenses on the basis of a phenomenon in which when a photosensitive resin is exposed to light in a desired pattern by the use of an aligner, unreacted monomers move from the unexposed regions to the exposed regions, resulting in a swell of the exposed regions (see, e.g., Journal of the Research Group in Microoptics Japanese Society of Applied Physics, Colloquium in Optics, Vol. 5, No. 2, pp. 118-123 (1987) and Vol. 6, No. 2, pp. 87-92(1988)).

On the upper surface of a supporting substrate, a photosensitive resin layer is formed. Thereafter, with the use of a separate shading mask, the upper surface of the photosensitive resin layer is illuminated with light from a mercury lamp or the like, so that the photosensitive resin layer is exposed to the light. As a result, the exposed portions of the photosensitive resin layer swell into the shape of convex lenses to form the light condensing layer having a plurality of microlens.

(4) A method for obtaining convex lenses wherein a photosensitive resin is exposed to light by a proximity exposure technique in which a photomask is not brought into contact with the resin, to cause a blur at the pattern edge, so that the amount of photochemical reaction products is distributed depending upon the degree of blurring at the pattern edge (see, e.g., JP 61-153602A).

(5) A method for generating a lens effect wherein a photosensitive resin is exposed to light with a particular intensity distribution to form a distribution pattern of refractive index depending upon the light intensity (see, e.g., JP 60-72927A and JP 60-166946A). The photosensitive compositions according to the invention can be used in any one of the above-mentioned methods to form microlens arrays using photocurable resin compositions.

A particular class of techniques concentrates on forming microlenses in thermoplastic resins like photoresist. An example is published by Popovic et al. in the reference SPIE 898, pp. 23-25 (1988). The technique, named reflow technique, comprises the steps of defining the lenses' footprint in a thermoplastic resin, e.g. by photolithography in a photosensitive resin like a photoresist, and subsequently heating this material above its reflow temperature. The surface tension draws the island of photoresist into a spherical cap with a volume equal to the original island before the reflow. This cap is a plano-convex microlens. Advantages of the technique are, amongst others, the simplicity, the reproducibility, and the possibility of integration directly on top of a light-emitting or light-detecting optoelectronic device.

In some cases, an overcoat layer is formed on the patterned lens units with a rectangular shape prior to reflowing to avoid a sagging of the island of the resin in the middle without reflow into a spherical cap in the reflow step. The overcoat acts as a permanent protective layer. The coating layer is also made of a photosensitive composition. Microlens arrays can also be fabricated by the use of a mold or a stamper as, for example, disclosed in EP0932256. A process of manufacturing the planar microlens array is as follows: a release agent is coated on a shaping surface of a stamper on which convex portions are densely arranged, and a photocurable synthetic resin material having a high refractive index is set on the shaping surface of the stamper. Next, the base glass plate is pushed onto the synthetic resin material, thereby spreading the synthetic resin material, and the synthetic resin material is cured by irradiating with ultraviolet radiation or by heating and is shaped to form the convex microlenses. Thereafter the stamper is peeled off. Then, a photocurable synthetic resin material having a low refractive index is additionally coated onto the convex microlenses as an adhesive layer and a glass substrate which is made into a cover glass plate is pushed onto the synthetic resin material, thereby spreading the same. The synthetic resin material is then cured and finally the planar microlens array is formed.

As disclosed in U.S. Pat. No. 5,969,867, a similar method using a mold is applied for the production of a prism sheet, which is used as a part of backlight units for color liquid crystal display panels to enhance the brightness. A prism sheet forming a prism row on one side is mounted on the light-emitting surface of the backlight. For fabricating a prism sheet, an active energy ray-curable composition is cast and spread in a lens mold which is made of metal, glass or resin and forms the lens shape of the prism row, etc., after which a transparent substrate sheet is placed onto it and active energy rays from an active energy ray-emitting source are irradiated through the sheet for curing. The prepared lens sheet is then released from the lens mold to obtain the lens sheet.

The active energy ray-curable composition used to form the lens section must have a variety of properties, including adhesion to the transparent substrate, and suitable optical characteristics.

Lenses at least with some photoresists in the prior art are not desirable for some applications since the optical transmittance in the blue end of the optical spectrum is poor. Because the photocurable compositions according to the invention have low yellowing properties, both thermally and photochemically, they are suitable for the production of microlens arrays as described above.

The novel radiation-sensitive compositions are also suitable for photo-lithographic steps used in the production process of plasma display panels (PDP), particularly for the imaging forming process of barrier rib, phosphor layer and electrodes.

The PDP is a planar display for displaying images and information by virtue of the emission of light by gas discharge. By the construction of panel and the method of operation, it is known in two types, i.e. DC (direct current) type and AC (alternating current) type.

By way of example, the principle of the DC type color PDP will be briefly explained. In the DC type color PDP, the space intervening between two transparent substrates (generally glass plates) is divided into numerous minute cells by latticed barrier ribs interposed between the transparent substrates. In the individual cells a discharge gas, such as He or Xe, is sealed. On the rear wall of each cell there is a phosphor layer which, on being excited by the ultraviolet light generated by the discharge of the discharge gas, emits visible light of three primary colors. On the inner faces of the two substrates, electrodes are disposed as opposed to each other across the relevant cells. Generally, the cathodes are formed of a film of transparent electroconductive material such as NESA glass. When a high voltage is applied between these electrodes formed on the fore wall and the rear wall, the discharge gas which is sealed in the cells induces plasma discharge and, by virtue of the ultraviolet light radiated consequently, incites the fluorescent elements of red, blue, and green colors to emit lights and effect the display of an image. In the full-color display system, three fluorescent elements severally of the three primary colors of red, blue, and green mentioned above jointly form one picture element.

The cells in the DC type PDP are divided by the component barrier ribs of a lattice, whereas those in the AC type PDP are divided by the barrier ribs which are arranged parallel to each other on the faces of the substrates. In either case, the cells are divided by barrier ribs. These barrier ribs are intended to confine the luminous discharge within a fixed area to preclude false discharge or cross talk between adjacent discharge cells and ensure ideal display.

The compositions according to the invention also find application for the production of one- or more-layered materials for the image recording or image reproduction (copies, reprography), which may be mono- or polychromatic. Furthermore the materials are suitable for color proofing systems. In this technology formulations containing microcapsules can be applied and for the image production the radiation curing can be followed by a thermal treatment. Such systems and technologies and their applications are for example disclosed in U.S. Pat. No. 5,376,459.

The compounds of the formula I are also suitable as photoinitiators in the holographic data storage application. Said photoinitiators generate radicals and initiate polymerization of monomer upon irradiation with blue laser radiation, suitable for holographic data storage. The wavelength range of the blue laser is 390-420 nm, preferably 400-410 nm and particularly 405 nm. Holographic storage systems (holographic recording media) are for example used to record and to retrieve a large amount of data with fast access time. The photoinitiators of the invention are for example in particular suitable for systems as described for example in WO03/021358. The holographic data storage system is preferably comprised of a matrix network of low-refractive index matrix precursors and high-refractive index photopolymerizable monomers.

The matrix precursor and photoactive monomer can be selected such that (a) the reaction by which the matrix precursor is polymerized during the cure is independent from the reaction by which the photoactive monomer will be polymerized during writing of a pattern, e.g. data, and (b) the matrix polymer and the polymer resulting from polymerization of the photoactive monomer (the photo polymer) are compatible with each other. The matrix is considered to be formed when the photorecording material, i.e. the matrix material plus the photoactive monomer, photoinitiator and/or additives, exhibits an elastic modulus of at least about 10⁵ Pa, generally about 10⁵ Pa to about 10⁹ Pa.

The media matrix is formed by in-situ polymerization which yields as cross-linked network in the presence of the photopolymerizable monomers which remain “dissolved” and unreacted. The matrix containing un-reacted, photopolymerizable monomers can also be formed by other means, for example by using a solid-resin matrix material in which the photoreactive, liquid monomer is homogeneously distributed. Then, monochromatic exposure generates the holographic pattern, which according to the light intensity distribution, polymerizes the photoreactive monomers in the solid pre-formed matrix. The unreacted monomers (where light intensity was at a minimum) diffuse through the matrix, producing a modulation of the refractive index that is determined by the difference between the refractive indices of the monomer and the matrix and by the relative volume fraction of the monomer. The thickness of the recording layer is in the range of several micrometers up to a thickness of one millimeter. Because of such thick holographic data storage layers it is required that the photoinitiator combines high photoreactivity with low absorbance, in order to render the layer transparent at the laser wavelength to assure that the extent of photopolymerization is as little as possible dependent on the exposure depth into the recording layer.

It was found that the photoinitiators of the present invention combine high reactivity with low absorbance at 405 nm and are suitable for this application. Dyes and sensitizers can also be added to the formulations. Suitable dyes and sensitizers for blue laser radiation are for example coumarines, xanthones, thioxanthones, see list above.

In particular relevant are thioxanthones, coumarins and benzophenones as mentioned under items 1., 2. and 3. in the list given above.

It was found that the photoinitiators allow photopolymerization of monomers in thick layers, such as required for holographic data storage, with high sensitivity and yield recording layers which are sensitive to blue laser radiation. The photoinitiators, when applied at a concentration of 2-8 wt % in the photosensitive layer of 20 micron thickness yield an absorbance of the layer which comprises the photoinitiator, of less than 0.4, preferably less than 0.2 at the laser wavelength.

The photoinitiators are in particular suitable for the preparation of optical articles (for example optical waveguides) or holographic recording media e.g. comprising a polymer and an organic photoinitiator as described above, having a maximum absorption at a UV wavelength in the range of 340-450 nm, wherein the refractive index contrast adjusted sensitivity is greater than 3×10⁻⁶ Δn/(mJ/cm²). For example, the polymer is formed by polymerizing a material comprising component 1 and component 2, wherein component 1 comprises a NCO-terminated pre-polymer and component 2 comprises a polyol. Component 1 is, for example, diphenylmethane diisocyanate, toluene diisocyanate, hexamethylene diisocyanate, a derivative of hexamethylene diisocyanate, a methylenebiscyclohexylisocyanate, a derivative of methylenebiscyclohexylisocyanate. Component 2 is for example a polyol of propylene oxide. Preferably, the photoactive monomer is an acrylate monomer. In such media the shrinkage induced by writing is usually less than 0.25%.

Photocuring further is of great importance for printings, since the drying time of the ink is a critical factor for the production rate of graphic products, and should be in the order of fractions of seconds. UV-curable inks are particularly important for screen printing and offset inks.

As already mentioned above, the novel mixtures are highly suitable also for producing printing plates. This application uses, for example, mixtures of soluble linear polyamides or styrene/butadiene and/or styrene/isoprene rubber, polyacrylates or polymethyl methacrylates containing carboxyl groups, polyvinyl alcohols or urethane acrylates with photopolymerizable monomers, for example acrylamides and/or methacrylamides, or acrylates and/or methacrylates, and a photoinitiator. Films and plates of these systems (wet or dry) are exposed over the negative (or positive) of the printed original, and the uncured parts are subsequently washed out using an appropriate solvent or aqueous solutions.

Another field where photocuring is employed is the coating of metals, in the case, for example, of the coating of metal plates and tubes, cans or bottle caps, and the photocuring of polymer coatings, for example of floor or wall coverings based on PVC.

Examples of the photocuring of paper coatings are the colourless varnishing of labels, record sleeves and book covers.

Also of interest is the use of the novel photoinitiators for curing shaped articles made from composite compositions. The composite compound consists of a self-supporting matrix material, for example a glass fibre fabric, or alternatively, for example, plant fibres [cf. K.-P. Mieck, T. Reussmann in Kunststoffe 85 (1995), 366-370], which is impregnated with the photocuring formulation.

The compositions and compounds according to the invention can be used for the production of holographies, waveguides, optical switches wherein advantage is taken of the development of a difference in the index of refraction between irradiated and unirradiated areas.

The use of photocurable compositions for imaging techniques and for the optical production of information carriers is also important. In such applications, as already described above, the layer (wet or dry) applied to the support is irradiated imagewise, e.g. through a photomask, with UV or visible light, and the unexposed areas of the layer are removed by treatment with a developer. Application of the photocurable layer to metal can also be carried out by electrodeposition. The exposed areas are polymeric through crosslinking and are therefore insoluble and remain on the support. Appropriate colouration produces visible images. Where the support is a metallized layer, the metal can, following exposure and development, be etched away at the unexposed areas or reinforced by electroplating. In this way it is possible to produce electronic circuits and photoresists. When used in image-forming materials the novel photoinitiators provide excellent performance in generating so called printout images, whereby a color change is induced due to irradiation. To form such printout images different dyes and/or their leuco form are used and examples for such print out image systems can be fount e.g. in WO96/41240, EP706091, EP511403, U.S. Pat. Nos. 3,579,339 and 4,622,286.

The novel photoinitiator is also suitable for a photopatternable composition for forming a dielectric layer of a multilayer layer circuit board produced by a sequential build-up process.

The invention, as described above, provides compositions, as well as a process, for producing pigmented and nonpigmented paints and varnishes, powder coatings, printing inks, printing plates, adhesives, pressure sensitive adhesives, dental compositions, gel coats, photoresists for electronics, etch resists, both liquid and dry films, solder resists, resists to manufacture color filters for a variety of display applications (a color filter resist contains pigments, pigments and dyes (i.e. hybrid systems) or dyes alone), resists to generate structures in the manufacturing processes of plasma-display panels (e.g. barrier rib, phosphor layer, electrode), electroluminescence displays and LCD (e.g. interlayer insulating layers, spacers, multi-spacers, microlens arrays), for encapsulating electrical and electronic components, for producing magnetic recording materials, micromechanical parts, waveguides, optical switches, plating masks, colour proofing systems, glass fibre cable coatings, screen printing stencils, three-dimensional objects by means of stereolithography, image recording materials for holographic recordings (e.g. for holographic data storage (HDS)), microelectronic circuits, decolorizing materials, formulations containing microcapsules, photoresist materials for a UV and visible laser direct imaging system and for forming dielectric layers in a sequential build-up layer of a printed circuit board, bank/pixel definition layers for OLED, sealants for LCD and OLED, insulation/passivation layers for LCD and OLED, insulation for metal wiring/transparent conductive films for touch panel, coatings for touch panel, decorative inks for touch panel, protective films for touch panel or etching resists for touch panel; wherein the process comprises irradiating a composition as described above with electromagnetic radiation in the range from 150 to 600 nm, or with electron beam or with X-rays.

Substrates used for photographic information recordings include, for example, films of polyester, cellulose acetate or polymer-coated papers; substrates for offset printing forms are specially treated aluminium, substrates for producing printed circuits are copper-clad laminates, and substrates for producing integrated circuits are, for example, silicon wafers. The layer thickness of the photosensitive layer for photographic materials and offset printing forms is generally from about 0.5μηι to 10μηι, while for printed circuits it is from 0.1μηι to about 100μηι. Following the coating of the substrates, the solvent is removed, generally by drying, to leave a coat of the photoresist on the substrate.

Coating of the substrates can be carried out by applying to the substrate a liquid composition, a solution or a suspension. The choice of solvents and the concentration depend principally on the type of composition and on the coating technique. The solvent should be inert, i.e. it should not undergo a chemical reaction with the components and should be able to be removed again, after coating, in the course of drying. Examples of suitable solvents are ketones, ethers and esters, such as methyl ethyl ketone, isobutyl methyl ketone, cyclopentanone, cyclohexanone, N-methylpyrrolidone, dioxane, tetrahydrofuran, 2-methoxyethanol, 2-ethoxyethanol, 1-methoxy-2-propanol, 1,2-dimethoxyethane, ethyl acetate, n-butyl acetate, ethyl 3-ethoxypropionate, 2-methoxypropylacetate, methyl-3-methoxypropionate, 2-heptanone, 2-pentanone, and ethyl lactate. The solution is applied uniformly to a substrate by means of known coating techniques, for example by spin coating, dip coating, knife coating, curtain coating, brushing, spraying, especially by electrostatic spraying, and reverse-roll coating, and also by means of electrophoretic deposition. It is also possible to apply the photosensitive layer to a temporary, flexible support and then to coat the final substrate, for example a copper-clad circuit board, or a glass substrate by transferring the layer via lamination.

The quantity applied (coat thickness) and the nature of the substrate (layer support) are dependent on the desired field of application. The range of coat thicknesses generally comprises values from about 0.1 μm to more than 100 μm, for example 0.1 μm to 1 cm, preferably 0.5 μm to 1000 μm. Following the coating of the substrates, the solvent is removed, generally by drying, to leave an essentially dry resist film of the photoresist on the substrate.

The photosensitivity of the novel compositions can extend in general from about 150 nm to 600 nm, for example 190-600 nm, (UV-vis region). Suitable radiation is present, for example, in sunlight or light from artificial light sources. Consequently, a large number of very different types of light sources are employed. Both point sources and arrays (“lamp carpets”) are suitable. Examples are carbon arc lamps, xenon arc lamps, low-, medium-, high- and super high-pressure mercury lamps, possibly with metal halide dopes (metal-halogen lamps), microwave-stimulated metal vapour lamps, excimer lamps, superactinic fluorescent tubes, fluorescent lamps, argon incandescent lamps, electronic flashlights, photographic flood lamps, light emitting diodes (LED, OLED), electron beams and X-rays. The distance between the lamp and the substrate to be exposed in accordance with the invention may vary depending on the intended application and the type and output of lamp, and may be, for example, from 2 cm to 150 cm. Laser light sources, for example excimer lasers, such as F2 excimer lasers at 157 nm exposure, KrF excimer lasers for exposure at 248 nm and ArF excimer lasers for exposure at 193 nm are also suitable. Lasers in the visible region can also be employed.

The term “imagewise” exposure includes both, exposure through a photomask comprising a predetermined pattern, for example a slide, a chromium mask, a stencil mask or a reticle, as well as exposure by means of a laser or light beam, which for example is moved under computer control over the surface of the coated substrate and in this way produces an image. Suitable UV laser exposure systems for the purpose are, for example, provided by Etec and Orbotech (DP-100™ DIRECT IMAGING SYSTEM). Other examples of laser light sources are, for example excimer lasers, such as F2 excimer lasers at 157 nm exposure, KrF excimer lasers for exposure at 248 nm and ArF excimer lasers for exposure at 193 nm. Further suitable are solid state UV lasers (e.g. Gemini from ManiaBarco, DI-2050 from PENTAX) and violet laser diodes with 405 nm output (DI-2080, DI-PDP from PENTAX). Lasers in the visible region can also be employed. And the computer-controlled irradiation can also be achieved by electron beams. It is also possible to use masks made of liquid crystals that can be addressed pixel by pixel to generate digital images, as is, for example, described by A. Bertsch, J. Y. Jezequel, J. C. Andre in Journal of Photochemistry and Photobiology A: Chemistry 1997, 107, p. 275-281 and by K.-P. Nicolay in Offset Printing 1997, 6, p. 34-37.

Following the imagewise exposure of the material and prior to development, it may be advantageous to carry out thermal treatment for a short time. After the development a thermal post bake can be performed to harden the composition and to remove all traces of solvents. The temperatures employed are generally 50-250° C., preferably 80-220° C.; the duration of the thermal treatment is in general between 0.25 and 60 minutes.

The invention therefore also provides a process for the photopolymerization of compounds containing ethylenically unsaturated double bonds, i.e. monomeric, oligomeric or polymeric compounds containing at least one ethylenically unsaturated double bond, which comprises adding to these compounds at least one photoinitiator of the formula I as described above and irradiating the resulting composition with electromagnetic radiation, in particular light of the wavelength 150 to 600 nm, in particular 190-600 nm, with electron beam, or with X-rays.

In other words, adding to these compounds compounds containing ethylenically unsaturated double bonds at least one photoinitiator of the formula I as described above and irradiating the resulting composition with electromagnetic radiation, in particular light of the wavelength 150 to 600 nm, in particular 190-600 nm, with electron beam, or with X-rays.

The invention further provides a coated substrate which is coated on at least one surface with a composition as described above. Interesting also is a process for the photographic production of relief images, in which a coated substrate as described above is subjected to imagewise exposure and then the unexposed portions are removed with a developer. Imagewise exposure may be effected by irradiating through a mask or by means of a laser or electron beam as already described above. Of particular advantage in this context is the laser beam exposure already mentioned above.

The compounds of the invention have a good thermal stability, low volatility, good storage stability and high solubility, and are also suitable for photopolymerizations in the presence of air (oxygen). Further, they cause only low discoloring in the compositions after photopolymerization.

The examples which follow illustrate the invention in more detail, without restricting the scope said examples only. Parts and percentages are, as in the remainder of the description and in the claims, by weight, unless stated otherwise. Where alkyl radicals having more than three carbon atoms are referred to in the examples without any mention of specific isomers, the n-isomers are meant in each case.

### SUBSTANCE EXAMPLES

### Example 1: Preparation of OE1

OE1 is prepared as the following scheme:

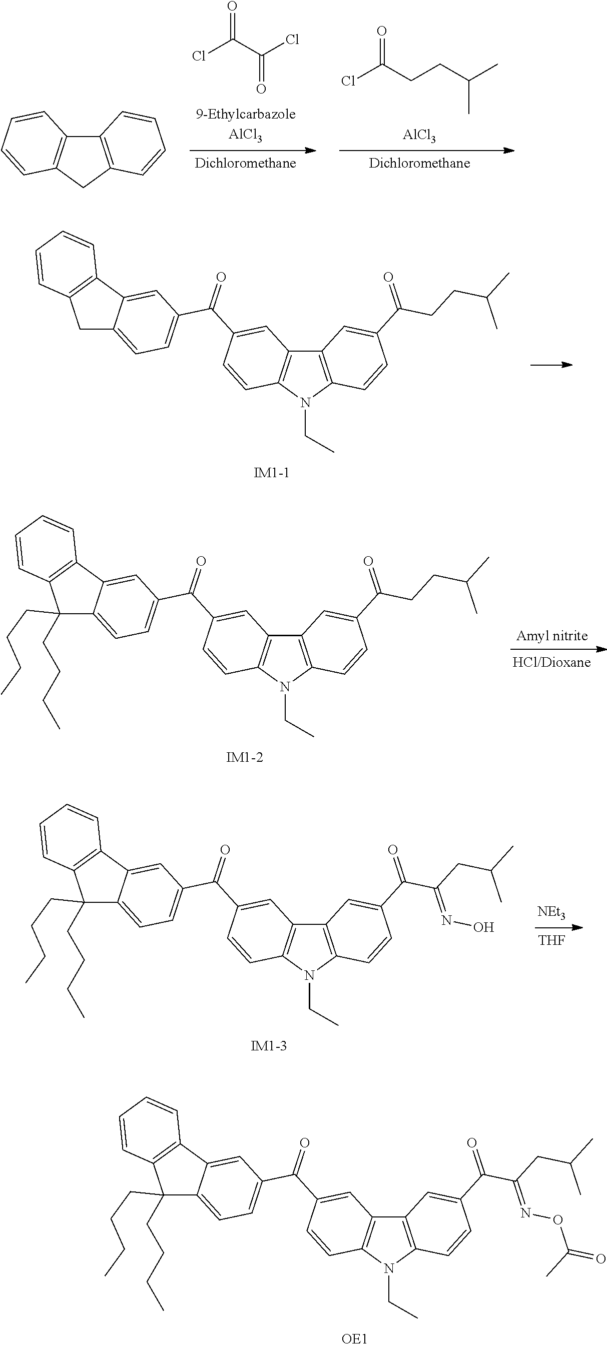

Preparation of IM1-1

To a solution of Fluorene (20.0 g) and Oxalyl Chloride (12.4 ml) in DCM (600 ml) was added aluminum chloride (16.8 g) at ice bath, the mixture was stirred for 1 h at room temperature. A solution of 9-ethylcarbazole in DCM was added to the reaction mixture at ice bath, the mixture was stirred for 1 h. Aluminum chloride (16.8 g) and 4-Methylvaleryl chloride (14.9 ml) was added to the reaction mixture at ice bath, the mixture was stirred for 1 h. The reaction mixture was quenched with ice water, extracted with DCM, washed with water, and dried over anhydrous MgSO4. The solvent was removed under reduced pressure and the crude product was purified by silica gel chromatography to give the product as a pale yellow solid (7.2 g).

Preparation of IM1-2

To a solution of IM1-1 (16.2 g) in DMSO (1300 ml) was added Potassium tert-butoxide (3.75 g) and 1-Iodobutane (3.8 ml), the mixture was stirred for 10 min at room temperature. Potassium tert-butoxide (3.75 g) and 1-Iodobutane (3.8 ml) were added to the reaction mixture, the mixture was stirred for 10 min at room temperature. The reaction mixture was quenched with ice water and the precipitate was collected by filtration. The solid was dissolved by ethyl acetate, washed with water, and dried over anhydrous MgSO4. The solvent was removed under reduced pressure to give the product as a pale yellow solid (20 g).

Preparation of IM1-3

To a solution of IM1-2 (19.1 g) and conc. HCl (2.7 ml) in DMF (120 ml) was added Amyl Nitrite (6.4 ml) at ice bath, the mixture was stirred for 15 h at room temperature. The reaction mixture was quenched with water, extracted with ethyl acetate, washed with brine, and dried over anhydrous MgSO₄. The solvent was removed under reduced pressure and the crude product was purified by silica gel chromatography to give the product as a pale yellow solid (7.8 g).

Preparation of OE1

To a solution of IM1-3 (4.9 g) and Triethylamine (1.1 ml) in DCM (100 ml) was added Acetyl Chloride (0.56 ml) at ice bath, the mixture was stirred for 15 min. The reaction mixture was quenched with water, extracted with DCM, and dried over anhydrous MgSO₄. The solvent was removed under reduced pressure to give the product as a pale yellow solid (4.5 g).

### Example 2: Preparation of OE2

OE2 is prepared as the following scheme:

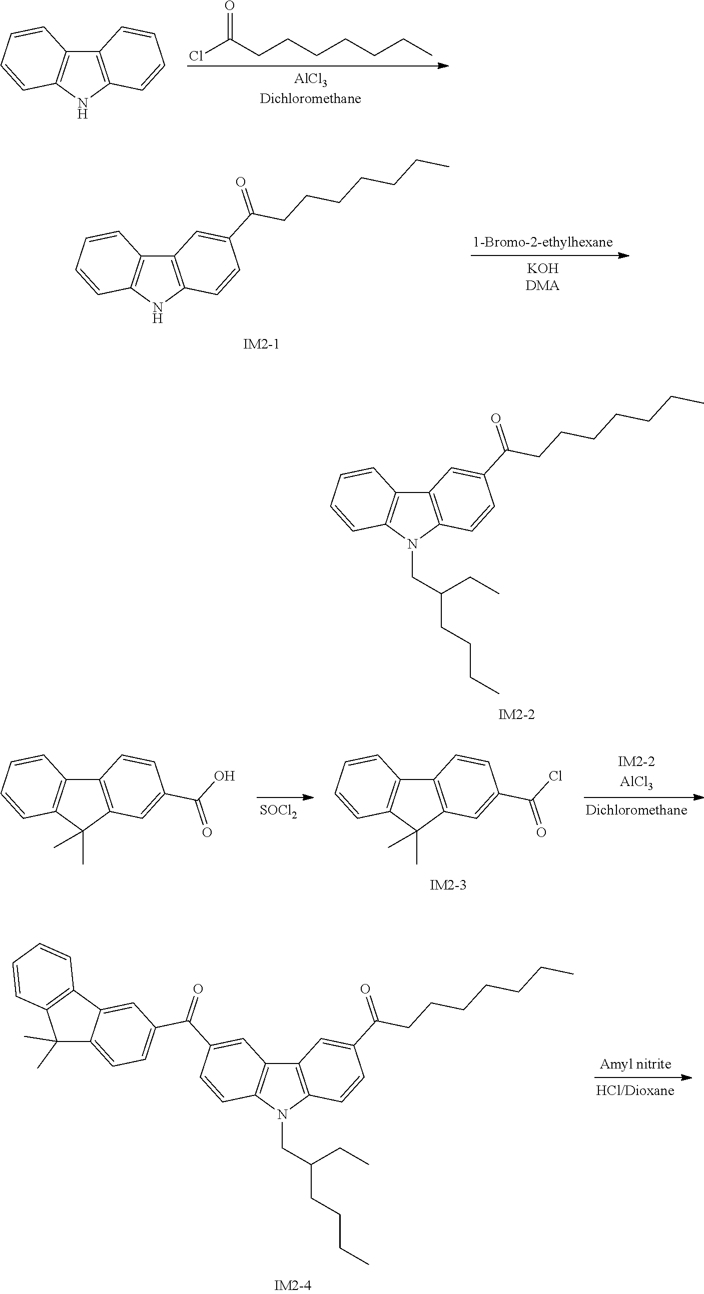

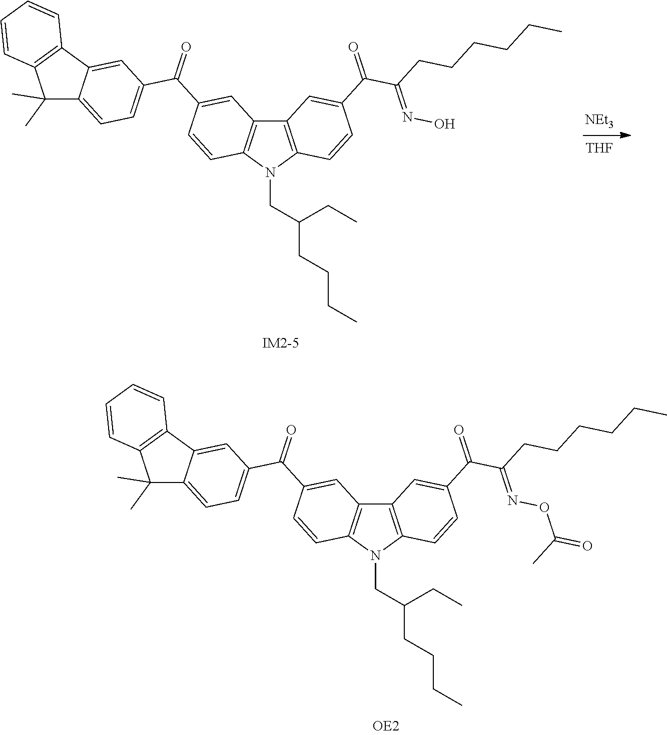

Preparation of IM2-1

To 10.023 g of 9-ethylcarbazole and 8.052 g of aluminum chloride in dichloromethane (130 ml) was added 9.759 g of n-octanoyl chloride and stirred at rt under N₂ flow conditions for 12 h. The reaction mixture was poured into ice-water and the precipitate was collected by filtration and dried in vacuo. The resulting solid was washed with mixture of hexane and dichloromethane, giving 8.628 g of IM2-1 as dark yellow sloid.

Preparation of IM2-2

To 2.078 g of IM2-1 and 1.377 g of 1-bromo-2-ethylhexane in DMA (10 ml) was added 0.475 g of potassium hydroxide, and stirred at room temperature for 12 h. Water is added to the reaction mixture and organic layer was extracted with ethyl acetate. The organic layer was washed with brine and dried over anhydrous sodium sulfate. The solvent was removed under vacuum, and the crude was purified via column chromatography with hexane and dichloromethane (75:25 v/v) as eluent to give 2.397 g of IM2-2 as yellow oil.

Preparation of IM2-4

To 1.266 g of 9,9-dimethylfluorene-2-carboxylic acid was added 2.0 ml of thionyl chloride and stirred at 80° C. for 6 h. After cooling to room temperature, thionyl chloride was removed under reduced pressure, then, 1.363 g of IM2-3 was obtained as yellow resin. To 2.592 g of IM2-2 and 1.816 g of aluminum chloride in dichloromethane (30 ml) was added 1.363 g of IM2-3 and stirred at rt under N₂ flow condition for 2 h. The reaction mixture was poured into ice-water and organic layer was separated. The organic layer was washed with brine and dried over anhydrous sodium sulfate. The solvent was removed under vacuum, and the crude was purified via column chromatography with hexane and dichloromethane (90:10 to 30:70 v/v) as eluent to give 1.448 g of IM2-4 as pale yellow resin.

Preparation of IM2-5

To 1.448 g of IM2-4 was added mixture of dichloromethane (20 ml) and 4 mol/l of hydrochloric acid in 1,4-dioxane solution (1.0 ml) at room temperature. Then, 0.284 g of amyl nitrite was added and stirred for 2 h. The solvent was removed under vacuum, and the crude was purified via column chromatography with hexane and dichloromethane (90:10 to 0:100 v/v) as eluent to give 0.716 g of IM2-5 as pale yellow resin.

Preparation of OE2

To 0.716 of IM2-5 and 0.126 of acetyl chloride in tetrahydrofuran (20 ml) was added 0.142 g of triethylamine at 0° C., then stirred at room temperature for 1 h. Water was added to the reaction mixture and organic layer was extracted with ethyl acetate. The organic layer was washed with brine and dried over anhydrous sodium sulfate. The solvent was removed under vacuum, and the crude was purified via column chromatography with hexane and dichloromethane (90:10 to 30:70 v/v) as eluent to give 0.496 g of OE2 as yellow resin.

Compound List and Physical Data Thereof

| Oxime ester (OE) | Structure | ¹H NMR in CDCl₃ |
|---|---|---|
| OE1 | 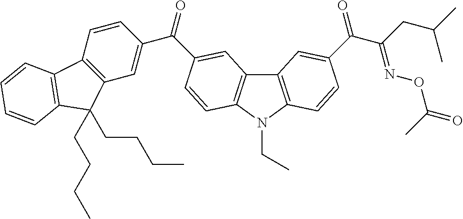 [SMILES: CCCCC1(CCCC)c2ccccc2-c2ccc(C(=O)c3ccc4c(c3)c3cc(C(=O)/C(CC(C)C)=N/OC(C)=O)ccc3n4CC)cc21] | 0.60-0.70 (m, 9H), 0.98 (d, 6H), 1.05-1.10 (m, 4H), 1.50-1.55 (m, 4H), 1.95-2.05 (m, 5H), 2.26 (s, 3H), 2.80 (d, 2H), 4.45 (q, 2H), 7.35-7.40 (m, 3H), 7.48 (d, 1H), 7.50 (d, 1H), 7.80-7.90 (m, 4H), 8.05 (dd, 1H), 8.34(dd, 1H), 8.72 (d, 1H), 8.91 (d, 1H) |
| OE2 | 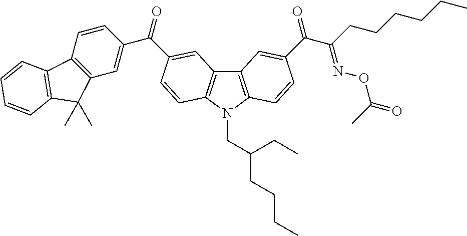 [SMILES: CCCCCC/C(=N\OC(C)=O)C(=O)c1ccc2c(c1)c1cc(C(=O)c3ccc4c(c3)C(C)(C)c3ccccc3-4)ccc1n2CC(CC)CCCC] | 0.84-0.89 (m, 6H), 0.95 (t, 3H), 1.26-1.56 (m, H), 2.03-2.11 (m, 1H), 2.25 (s, 3H), 2.84 (t, 2H), 4.26 (m, 2H), 7.93-7.43, (m, 2H), 7.47- 7.52 (m, 3H), 7.82-7.85 (m, 3H), 8.01 (s, 1H), 8.09 (dd, 1H), 8.29 (dd, 1H), 8.69 (d, 1H), 8.84 (d, 1H) |

### APPLICATION EXAMPLES

Sensitivity Test: A

Preparation of Color Filter Resist (Blue)

A blue pigment dispersion is prepared by mixing the following components and dispersing them by using a Paint conditioner (SKANDEX).

Blue Dispersion

| | |
|---|---|
| 5.6 parts by weight | blue pigment (PB15:6, Blue E provided by |
| | Toyo Ink) |
| 0.6 parts by weight | violet pigment (PV23, Cromophtal Violet GA |
| | provided by BASF) |
| 2.6 parts by weight | dispersant (EFKA6745 provided by BASF) |
| 0.4 parts by weight | synergist (Solsperse S5000 provided by |
| | Lubrizol) |
| 19.6 parts by weight | solvent (PGMEA) |

Color filter resists (blue) are prepared by further adding the following components to the above dispersion prepared.

| | |
|---|---|
| 6.0 parts by weight | alkaline developable binder, 37.8% solution |
| | (Ripoxy SPC-2000, provided by Shoko |
| | Highpolymer) |
| 41.9 parts by weight | solvent (PGMEA) |
| 5.0 parts by weight | multifunctional acrylate (DPHA, provided by |
| | Sigma-Aldrich) |
| 0.7 parts by weight | Photoinitiator |

All operations are carried out under yellow light. The compositions are applied to a glass plate using a spincoater. The solvent is removed by heating at 80° C. for 10 minutes in a convection oven. The thickness of the dry film is approximately 1.2 μm. A stepwedge pattern mask with 9 linear steps and 9 logarithmic steps of different optical density (Edumund Optics, EIA GrayScale Pattern Slide) is placed on the resist directly. Exposure is carried out using a 250 W super high pressure mercury lamp (USHIO, USH-250BY) at a distance of 15 cm. A total exposure dose measured by an optical power meter (ORC UV Light Measure Model UV-M02 with UV-35 detector) on the glass filter is 500 mJ/cm². After exposure, the exposed film is developed with an alkaline solution (5% aqueous solution of DL-A4, YOKOHAMA OILS & FATS) for 10 seconds after break time at 28° C. by using a spray type developer (AD-1200, MIKASA). Break time is the development time of the unexposed region. The necessary UV dose for full curing (i.e. the pattern is not dissolved with the alkaline solution) is calculated from the residual thickness of each step after development. The smaller the value of the dose, the more sensitive is the tested initiator. The results are listed in Table 1.

Transparent Test: B

All operations are carried out under yellow light. The compositions are applied to a glass plate using a spincoater. The film is measured transmittance from 350 nm to 780 nm after drying, exposure and baking using UV-VIS spectrometer (Shimazu, UV-2550). The solvent is removed by drying at 80° C. for 10 minutes in a convection oven. Exposure is carried out using a 250 W super high pressure mercury lamp (USHIO, USH-250BY) at a distance of 15 cm. A total exposure dose measured by an optical power meter (ORC UV Light Measure Model UV-M02 with UV-35 detector) on the glass filter is 50 mJ/cm². After exposure, the exposed film is baked at 230° C. for 30 minutes in a forced convention oven. ΔE*ab at 0.094 of y is calculated from transmittance of the film. ΔE*ab means the difference of transmittance of the film after drying and of that after baking 230° C. for 30 minutes and smaller ΔE*ab value indicates less discoloration. The results are listed in Table 1.

TABLE 1
Sensitivity and discoloration

| Example No. | PI | Sensitivity [mJ cm⁻²] | ΔE*ab |
|---|---|---|---|
| Example B1 | OE1 | 50.6 | 2.62 |
| Example B2 | OE2 | 50.6 | 2.69 |
| Comparative example 1 | OE-R1 | 366 | 3.58 |
| Comparative example 2 | OE-R2 | 206.3 | 4.76 |

The following photoinitiators were used in comparative examples 1 and 2:

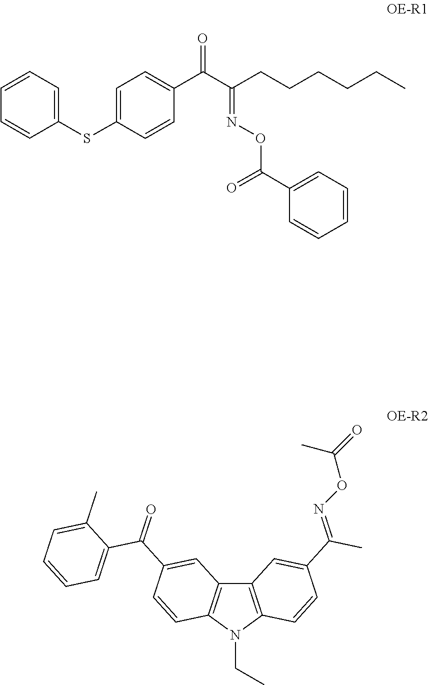 [SMILES: CCCCCC/C(=N\OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21]

As shown in table 1, the compounds of the present invention show significantly improved photosensitivity and at the same time reduced discoloration.

1. Compounds of the formula I 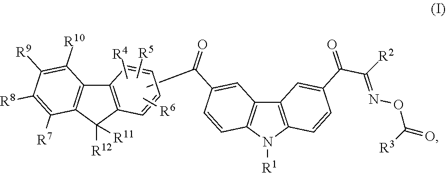
wherein
R¹ is C₁-C₂₀alkyl which is unsubstituted or substituted by one or more halogen, OR¹⁴, SR¹⁵, COOR¹⁴, CONR¹⁶R¹⁷, NR¹⁶R¹⁷, PO(OCₖH₂ₖ₊₁)₂; or by 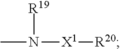
R¹ is C₂-C₂₀alkyl which is interrupted by one or more O, CO, S, SO, SO₂, C(O)O, OC(O), phenylene, naphthylene or NR¹⁸, wherein the interrupted C₂-C₂₀alkyl is unsubstituted or substituted by one or more halogen, OR¹⁴, SR¹⁵, COOR¹⁴, CONR¹⁶R¹⁷, NR¹⁶R¹⁷;
R¹ is C₂-C₁₂alkenyl which is uninterrupted or is interrupted by one or more O, CO, S, SO, SO₂, C(O)O, OC(O), phenylene, naphthylene or NR¹⁸, wherein the uninterrupted or interrupted C₂-C₁₂alkenyl are unsubstituted or are substituted by one or more halogen, OR¹⁴, SR¹⁵, COOR¹⁴, CONR¹⁶R¹⁷, NR¹⁶R¹⁷;
R¹ is C₄-C₈cycloalkenyl, C₂-C₁₂alkinyl, or C₃-C₁₀cycloalkyl which is uninterrupted or is interrupted by one or more O, CO, S, SO, SO₂, C(O)O, OC(O), phenylene, naphthylene or NR¹⁸; or
R¹ is C₆-C₂₀aryl or C₃-C₂₀heteroaryl each of which is unsubstituted or substituted by one or more phenyl, halogen, C₁-C₄haloalkyl, CN, NO₂, 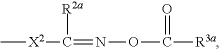
COR^{3a}, OR¹⁴, SR¹⁵, NR¹⁶R¹⁷, or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷, or each of which is substituted by C₃-C₁₀cycloalkyl or by C₃-C₁₀cycloalkyl which is interrupted by one or more O, S, CO, or NR¹⁸;
R² is hydrogen, CN, COR^{3a}, COOR¹⁴, CONR¹⁶R¹⁷, NO₂, PO(OR^{3a})₂ or S(O)ₘR^{3a}; or
R² is C₁-C₂₀alkyl which is unsubstituted or substituted by one or more substituents selected from the group consisting of halogen, OR¹⁴, SR¹⁵, NR¹⁶R¹⁷, CN, COOR¹⁴, CONR¹⁶R¹⁷, PO(OR^{3a})₂, S(O)ₘR^{3a}, 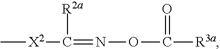
C₃-C₈cycloalkyl which is uninterrupted or interrupted by one or more O, S, CO or NR¹⁶,
C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵, NR¹⁶R¹⁷, PO(OR^{3a})₂ or S(O)ₘR^{3a}; or
R² is C₂-C₂₀alkyl interrupted by one or more O, S, NR¹⁶, CO, SO or SO₂, which interrupted C₂-C₂₀alkyl is unsubstituted or substituted by one or more C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵ or NR¹⁶R¹⁷; or
R² is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR¹⁶ or COOR¹⁴; or
R² is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR¹⁴, SR¹⁵, NR¹⁶R¹⁷, COOR¹⁴, COR^{3a} CONR¹⁶R¹⁷, PO(OR^{3a})₂, S(O)ₘR^{3a}, 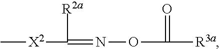
C₁-C₂₀alkyl which is unsubstituted or substituted by one or more halogen, COOR^{14a}, CONR¹⁶R¹⁷, phenyl, C₃-C₈cycloalkyl, C₃-C₂₀heteroaryl, OR¹⁴, SR¹⁵ or NR¹⁶R¹⁷, C₂-C₂₀alkyl which is interrupted by one or more O, S or NR¹⁶,
phenyl, naphthyl, benzoyl and naphthoyl, which phenyl, naphthyl, benzoyl or naphthoyl is unsubstituted or substituted by one or more OR¹⁴, SR¹⁵ or NR¹⁶R¹⁷;
R^{2a} is hydrogen, CN, COR^{3a}, COOR^{14a}, CONR^{16a}R^{17a}, NO₂, PO(OR^{3a})₂ or S(O)ₘR^{3a}; or
R^{2a} is C₁-C₂₀alkyl which is unsubstituted or substituted by one or more substituents selected from the group consisting of
halogen, OR^{14a}, SR^{15a}, NR^{16a}R^{17a}, CN, COOR^{14a}, CONR^{16a}R^{17a}, PO(OR^{3a})₂, S(O)ₘR^{3a}, C₃-C₈cycloalkyl which is uninterrupted or interrupted by one or more O, S, CO or NR^{16a},
C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or
R^{2a} is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or
R^{2a} is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or
R^{2a} is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a},
C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a};
C₂-C₂₀alkyl which is interrupted by one or more O, S or NR^{16a},
phenyl, naphthyl, benzoyl and naphthoyl, each of which phenyl, naphthyl, benzoyl or naphthoyl is unsubstituted or substituted by one or more OR^{14a}, SR^{15a} or NR^{16a}R^{17a};
R³ is hydrogen, C₁-C₂₀alkyl which is unsubstituted or substituted by one or more substituents selected from the group consisting of halogen, OR¹⁴, SR¹⁵, NR¹⁶R¹⁷, CN, COOR¹⁴, CONR¹⁶R¹⁷, C₃-C₈cycloalkyl, C₃-C₈cycloalkyl which is interrupted by one or more O, S, CO or NR¹⁶, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵ or NR¹⁶R¹⁷; or
R³ is C₂-C₂₀alkyl interrupted by one or more O, S, NR¹⁶, CO, SO or SO₂, which interrupted C₂-C₂₀alkyl is unsubstituted or substituted by one or more C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵ or NR¹⁶R¹⁷; or
R³ is C₂-C₁₂alkenyl or C₃-C₂₀cycloalkyl, each of which is uninterrupted or interrupted by one or more O, S, CO, NR¹⁶ or COOR¹⁴; or
R³ is C₆-C₂₀aryl or C₃-C₂₀heteroaryl, each of which is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, COR^{3a} or SO₂—R^{3a}; or
R³ is C₁-C₂₀alkoxy, which is unsubstituted or substituted by one or more C₁-C₁₀alkyl, C₁-C₄haloalkyl, halogen, phenyl, C₁-C₂₀alkylphenyl or C₁-C₈alkoxyphenyl; or
R³ is C₂-C₂₀alkoxy, which is interrupted by one or more O, S, NR¹⁶, CO, SO or SO₂; or
R³ is C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, (CO)R^{3a} or SO₂R^{3a};
R^{3a} is hydrogen, C₁-C₂₀alkyl, C₁-C₄haloalkyl which is unsubstituted or substituted by one or more substituents selected from the group consisting of halogen, OR^{14a}, SR^{15a}, NR^{16a}R^{17a}, CN, COOR^{14a}, CONR^{16a}R^{17a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a} or NR^{16a}R^{17a}; or
R^{3a} is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)—(C₁-C₈alkyl), COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or
R^{3a} is C₂-C₁₂alkenyl or C₃-C₂₀cycloalkyl, each of which is uninterrupted or interrupted by one or more O, S, CO, NR^{16a} or COOR^{14a}; or
R^{3a} is C₆-C₂₀aryl or C₃-C₂₀heteroaryl, which C₆-C₂₀aryl or C₃-C₂₀heteroaryl is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR^{14a}, SR^{15a}, NR^{16a}R^{17a}, COOR^{14a}, (CO)—(C₁-C₈alkyl), benzoyl or SO₂—(C₁-C₄haloalkyl); or
R^{3a} is C₁-C₂₀alkoxy, which is unsubstituted or substituted by one or more C₁-C₁₀alkyl, C₁-C₄haloalkyl, halogen, phenyl, C₁-C₂₀alkylphenyl or C₁-C₈alkoxyphenyl; or
R^{3a} is C₂-C₂₀alkoxy, which is interrupted by one or more O, S, NR^{16a}, CO, SO or SO₂; or
R^{3a} is C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR^{14a}, SR^{15a}, NR^{16a}R^{17a}, COOR^{14a}, (CO)(C₁-C₈alkyl), SO₂—(C₁-C₄haloalkyl), benzoyl, or 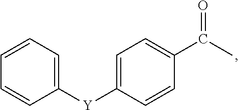
which benzoyl or 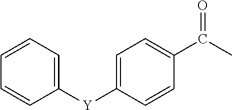
is unsubstituted or substituted by 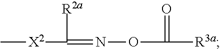
R⁴, R⁵, R⁶, R⁷, R⁸, R⁹ and R¹⁰ independently of each other are hydrogen, halogen, C₁-C₂₀alkyl, C₁-C₂₀haloalkyl, C₆-C₂₀aryl, C₁-C₂₀alkoxy, C₆-C₂₀arylC₁-C₂₀alkyl, C₃-C₁₀cycloalkyl, amino, CN, NO₂, O(CO)C₁-C₂₀alkyl, O(CO)C₆-C₁₀aryl, 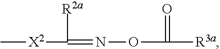
COR¹³, COOR¹⁴, or
R⁴ and R⁵ or R⁵ and R⁶, R⁷ and R⁸, R⁸ and R⁹, and R⁹ and R¹⁰ are 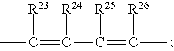
R¹¹ and R¹² independently of each other are hydrogen, halogen, C₁-C₃₀alkyl, C₆-C₂₀aryl, C₁-C₂₀alkoxy, C₆-C₂₀arylC₁-C₂₀alkyl, hydroxyC₁-C₂₀alky, hydroxyC₁-C₂₀alkoxyC₁-C₂₀alkyl, C₃-C₂₀cycloalkyl, C₃-C₂₀heterocycloalkyl, C₃-C₂₀cycloalkylC₁-C₂₀alkyl, C₂-C₃₀alkenyl, C₂-C₃₀alkynyl; or
R¹¹ and R¹² are connected to each other to form a C₂-C₅alkylene ring, C₂-C₅alkenylene ring, or C₂-C₅alkynylene ring;
R¹³ is C₆-C₂₀aryl or C₃-C₂₀heteroaryl each of which is unsubstituted or substituted by one or more phenyl, halogen, C₁-C₄haloalkyl, CN, NO₂, OR¹⁴, SR¹⁵, NR¹⁶R¹⁷, or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷;
R¹⁴ is hydrogen, COR^{3a}, COOR^{14a}, CONR¹⁶R¹⁷, C₁-C₂₀alkyl which is unsubstituted or substituted by one or more substituents selected from the group consisting of halogen, OR^{14a}, SR^{15a}, NR^{16a}R^{17a}, CN, COOR^{14a}, CONR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or
R¹⁴ is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or
R¹⁴ is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or
R¹⁴ is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a}, 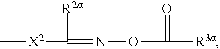
C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a}, C₂-C₂₀alkyl which is interrupted by one or more O, S or NR^{16a};
phenyl, naphthyl, benzoyl and naphthoyl, which phenyl, naphthyl, benzoyl or naphthoyl is unsubstituted or substituted by one or more OR^{14a}, SR^{15a} or NR^{16a}R^{17a}; or
R¹⁴ together with one of the carbon atoms of R₂ or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a} or NR^{16a}R^{17a}, COR^{3a}, NO₂, halogen, C₁-C₄-haloalkyl, CN, phenyl, C₁-C₂₀alkylphenyl, C₁-C₈alkoxyphenyl, 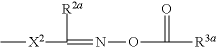
or C₃-C₂₀cycloalkyl which is uninterrupted or interrupted by one or more O, S, CO or NR^{16a};
R^{14a} is hydrogen, (CO)O(C₁-C₈alkyl) or CON(C₁-C₈alkyl)₂; or
R^{14a} is C₁-C₂₀alkyl which is unsubstituted or substituted by one or more substituents selected from the group consisting of halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl and C₃-C₈cycloalkyl which is interrupted by one or more O; or
R^{14a} is C₂-C₂₀alkyl interrupted by one or more O, S, N(C₁-C₈alkyl), CO, SO or SO₂, which interrupted C₂-C₂₀alkyl is unsubstituted or substituted by one or more C₃-C₈cycloalkyl, OH, SH, O(CO)(C₁-C₈alkyl), (CO)O(C₁-C₈alkyl), (CO)N(C₁-C₈alkyl)₂, C₆-C₂₀aryl, C₃-C₂₀het¬eroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or
R^{14a} is C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or
R^{14a} is C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₃alkyloxy, phenoxy, C₁-C₈alkylsulfanyl, phenylsulfanyl, N(C₁-C₈alkyl)₂, diphenylamino, (CO)O(C₁-C₈alkyl), (CO)C₁-C₈alkyl or (CO)N(C₁-C₈)₂, phenyl or benzoyl; or
R^{14a} is C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino;
R¹⁵ is hydrogen, C₁-C₂₀alkyl which is unsubstituted or substituted by one or more substituents selected from the group consisting of halogen, OR^{14a}, SR^{15a} or NR^{16a}R^{17a}, CN, COOR^{14a}, CONR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or
R¹⁵ is C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, CONR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a}, or NR^{16a}R^{17a}; or
R¹⁵ is C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or
R¹⁵ is C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a} CONR^{16a}R^{17a}, PO(OR^{3a})₂, S(O)ₘR^{3a}, 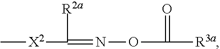
C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a} or NR^{16a}R^{17a}, C₂-C₂₀alkyl which is interrupted by one or more O, S or NR^{16a},
phenyl, naphthyl, benzoyl and naphthoyl, which phenyl, naphthyl, benzoyl or naphthoyl is unsubstituted or substituted by OR^{14a}, SR^{15a} or NR^{16a}R^{17a}; or
R¹⁵ together with one of the carbon atoms of R₂ or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄-haloalkyl, CN, phenyl, C₁-C₂₀alkylphenyl, C₁-C₈alkoxyphenyl, 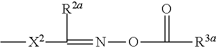
or C₃-C₂₀cycloalkyl which is uninterrupted or interrupted by one or more O, S, CO or NR^{16a};
R^{15a} is hydrogen, C₁-C₂₀alkyl which is unsubstituted or substituted by one or more substituents selected from the group consisting of halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl and C₃-C₈cycloalkyl which is interrupted by one or more O; or
R^{15a} is C₂-C₂₀alkyl interrupted by one or more O, S, N(C₁-C₈alkyl), CO, SO or SO₂, which interrupted C₂-C₂₀alkyl is unsubstituted or substituted by one or more C₃-C₈cycloalkyl, OH, SH, O(CO)(C₁-C₈alkyl), (CO)O(C₁-C₈alkyl), (CO)N(C₁-C₈alkyl)2, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or
R^{15a} is C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or
R^{15a} is C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₃alkyloxy, phenoxy, C₁-C₈alkylsulfanyl, phenylsulfanyl, N(C₁-C₈alkyl)₂, diphenylamino, (CO)O(C₁-C₈alkyl), (CO)C₁-C₈alkyl, (CO)N(C₁-C₈alkyl)₂, phenyl or benzoyl; or
R^{15a} is C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino;
R¹⁶ and R¹⁷ independently of each other are hydrogen, S(O)ₘR^{3a}, O(CO)R^{3a}, COR^{3a} or CONR^{16a}R^{17a}; or
R¹⁶ and R¹⁷ independently of each other are C₁-C₂₀alkyl which is unsubstituted or substituted by one or more substituents selected from the group consisting of halogen, OR^{14a}, SR^{15a}, NR^{16a}R^{17a}, C_{N} COOR^{14a}, COR^{16a}R^{17a}, PO(OR^{3a})₂, S(O)ₘR^{3a}, C₃-C₈cycloalkyl which is uninterrupted or interrupted by one or more O, S, CO or NR^{16a}, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR^{14a}, SR^{15a}, NR^{16a}R^{17a}, PO(OR^{3a})₂ or S(O)ₘR^{3a}; or
R¹⁶ and R¹⁷ independently of each other are C₂-C₂₀alkyl interrupted by one or more O, S, NR^{16a}, CO, SO or SO₂, which interrupted C₂-C₂₀alkyl is unsubstituted or substituted by one or more C₃-C₈cycloalkyl, OH, SH, O(CO)R^{3a}, COOR^{14a}, COR^{16a}R^{17a}, C₆-C₂₀aryl, C₃-C₂₀heteroaryl, C₆-C₂₀aroyl or C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR^{14a}, SR^{15a} or NR^{16a}R^{17a}; or
R¹⁶ and R¹⁷ independently of each other are C₂-C₁₂alkenyl or C₃-C₂₀cycloalkyl, which C₂-C₁₂alkenyl or C₃-C₂₀cycloalkyl is uninterrupted or interrupted by one or more O, S, CO, NR^{16a} or COOR^{14a}; or
R¹⁶ and R¹⁷ independently of each other are C₆-C₂₀aryl or C₃-C₂₀heteroaryl, which C₆-C₂₀aryl or C₃-C₂₀heteroaryl is unsubstituted or substituted by one or more substituents selected from the group consisting of halogen, CN, NO₂, OR^{14a}, SR^{15a}, NR^{16a}R^{17a}, COOR^{14a}, COR^{3a}, CONR^{16a}R^{17a}, PO(OR^{3a})₂, S(O)ₘR^{3a}, 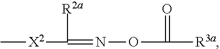
C₁-C₂₀alkyl which is unsubstituted or substituted by one or more halogen, COOR^{14a}, CONR^{16a}R^{17a}, phenyl, C₃-C₈cycloalkyl, C₃-C₂₀heteroaryl, OR^{14a}, SR^{15a}, or NR^{16a}R^{17a}, C₂-C₂₀alkyl which is interrupted by one or more O, S or NR^{16a},
phenyl, naphthyl, benzoyl and naphthoyl, which phenyl, naphthyl, benzoyl or naphthoyl is unsubstituted or substituted by OR^{14a}, SR^{15a}, or NR^{16a}R^{17a}; or
R¹⁶ and R¹⁷ independently of each other are C₁-C₂₀alkoxy, which is unsubstituted or substituted by one or more halogen, phenyl, C₁-C₈alkylphenyl or C₁-C₈alkoxyphenyl; or
R¹⁶ and R¹⁷ independently of each other are C₂-C₂₀alkoxy, which is interrupted by one or more O, S, NR^{16a}, CO, SO or SO₂; or
R¹⁶ and R¹⁷ independently of each other are C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₈alkyl, C₁-C₄haloalkyl, phenyl, C₁-C₈alkylphenyl, C₁-C₈alkoxyphenyl, CN, NO₂, OR^{14a}, SR^{15a}, NR^{16a}R^{17a}, COOR^{14a}, COR^{3a} or SO₂R^{3a}; or
R¹⁶ together with one of the carbon atoms of R² or M forms a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄-haloalkyl, CN, phenyl, C₁-C₂₀alkylphenyl, C₁-C₈alkoxyphenyl, 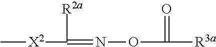
or C₃-C₂₀cycloalkyl which is uninterrupted or interrupted by one or more O, S, CO or NR^{16a}; or
R¹⁶ and R¹⁷ together with the N-atom to which they are attached form a 5- or 6-membered saturated or unsaturated ring which is uninterrupted or interrupted by O, S or NR^{16a}, and which 5- or 6-membered saturated or unsaturated ring is unsubstituted or substituted by one or more C₁-C₂₀alkyl, OR^{14a}, SR^{15a}, NR^{16a}R^{17a}, COR^{3a}, NO₂, halogen, C₁-C₄-haloalkyl, CN, phenyl, 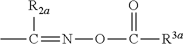
or C₃-C₂₀cycloalkyl which is uninterrupted or interrupted by one or more O, S, CO or NR^{16a};
R^{16a} and R^{17a} independently of each other are hydrogen, C₁-C₂₀alkyl, C₁-C₄haloalkyl, S(O)ₘ(C₁-C₈alkyl), O(CO)(C₁-C₈alkyl), (CO)(C₁-C₈alkyl), (CO)O(C₁-C₈alkyl) or CON(C₁-C₈alkyl)₂; or
R^{16a} and R^{17a} independently of each other are C₁-C₂₀alkyl substituted by one or more halogen, OH, SH, CN, C₃-C₈alkenoxy, OCH₂CH₂CN, OCH₂CH₂(CO)O(C₁-C₈alkyl), O(CO)(C₁-C₈alkyl), O(CO)(C₂-C₄)alkenyl, O(CO)phenyl, (CO)OH, (CO)O(C₁-C₈alkyl), C₃-C₈cycloalkyl, SO₂(C₁-C₄haloalkyl), O(C₁-C₄haloalkyl), phenyl, C₁-C₈alkylphenyl, C₁-C₈alkoxyphenyl, C₃-C₈cycloalkyl or C₃-C₈cycloalkyl which is interrupted by one or more O; or
R^{16a} and R^{17a} independently of each other are C₂-C₂₀alkyl interrupted by one or more O, S, N(C₁-C₈alkyl), CO, SO or SO₂, which interrupted C₂-C₂₀alkyl is unsubstituted or substituted by C₃-C₈cycloalkyl, OH, SH, O(CO)(C₁-C₈alkyl), (CO)O(C₁-C₈alkyl), (CO)N(C₁-C₈alkyl)₂, C₆-C₂₀aryl, C₃-C₂₀heteroaryl, C₆-C₂₀aroyl or C₃-C₂₀heteroaryl-carbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, C₁-C₈alkoxy, C₁-C₈alkylsulfanyl or N(C₁-C₈alkyl)₂; or
R^{16a} and R^{17a} independently of each other are C₂-C₁₂alkenyl or C₃-C₈cycloalkyl, which C₂-C₁₂alkenyl or C₃-C₈cycloalkyl is uninterrupted or interrupted by one or more O, S, CO, N(C₁-C₈alkyl) or COO(C₁-C₈alkyl); or
R^{16a} and R^{17a} independently of each other are C₆-C₂₀aryl, C₃-C₂₀heteroaryl, C₆-C₂₀aroyl or C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, CN, NO₂, OH, C₁-C₈alkyl, C₁-C₄haloalkyl, C₁-C₈alkoxy, phenyl-C₁-C₃alkyloxy, phenoxy, C₁-C₈alkylsulfanyl, phenylsulfanyl, N(C₁-C₈alkyl)₂, diphenylamino, (CO)O(C₁-C₈alkyl), (CO)C₁-C₈alkyl, (CO)N(C₁-C₈alkyl)₂, phenyl or benzoyl; or
R^{16a} and R^{17a} independently of each other are C₁-C₂₀alkanoyl or C₃-C₁₂alkenoyl, which C₁-C₂₀alkanoyl or C₃-C₁₂alkenoyl is unsubstituted or substituted by one or more halogen, phenyl, C₁-C₈alkylphenyl, C₁-C₈alkoxyphenyl, OH, C₁-C₈alkoxy, phenoxy, C₁-C₈alkylsulfanyl, phenylsulfanyl, N(C₁-C₈alkyl)₂ or diphenylamino; or
R^{16a} and R^{17a} independently of each other are C₁-C₂₀alkoxy, which is unsubstituted or substituted by one or more halogen, phenyl, C₁-C₈alkylphenyl or C₁-C₈alkoxyphenyl; or
R^{16a} and R^{17a} independently of each other are C₂-C₂₀alkoxy, which is interrupted by one or more O, S, N(C₁-C₈alkyl), CO, SO or SO₂; or
R^{16a} and R^{17a} independently of each other are C₆-C₂₀aryloxy or C₃-C₂₀heteroaryloxy, which C₆-C₂₀aryloxy or C₃-C₂₀heteroaryloxy is unsubstituted or substituted by one or more halogen, C₁-C₈alkyl, C₁-C₄haloalkyl, phenyl, C₁-C₈alkylphenyl, C₁-C₈alkoxyphenyl, CN, NO₂, C₁-C₈alkoxy, C₁-C₈alkylsulfanyl, N(C₁-C₈alkyl)₂, CO(OC₁-C₈alkyl), (CO)(C₁-C₈alkyl) or SO₂—(C₁-C₈alkyl); or
R^{16a} and R^{17a} together with the N-atom to which they are attached form a 5- or 6-membered saturated or unsaturated ring which is uninterrupted or interrupted by O, S or N(C₁-C₈alkyl), and which 5- or 6-membered saturated or unsaturated ring is unsubstituted or substituted by one or more C₁-C₈alkyl, C₁-C₈alkoxy, C₁-C₈alkylsulfanyl, N(C₁-C₈alkyl)₂, NO₂, halogen, C₁-C₄haloalkyl, CN, phenyl or C₃-C₂₀cycloalkyl which is uninterrupted or interrupted by one or more O, S, CO or N(C₁-C₈alkyl);
R¹⁸ is hydrogen, C₁-C₂₀alkyl, C₁-C₄haloalkyl, C₂-C₂₀alkyl which is interrupted by one or more O, CO, C(O)O, or OC(CO), or is phenyl-C₁-C₄alkyl, C₃-C₈cycloalkyl which is uninterrupted or is interrupted by one or more O, CO, C₁-C₂₀alkyl, halogen, C₁-C₄haloalkyl, OR¹⁴, SR¹⁵, NR¹⁶R¹⁷ or 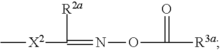
or
R¹⁸ is COR²¹; or
R¹⁸ is phenyl or naphthyl both of which are unsubstituted or substituted by one or more C₁-C₂₀alkyl, halogen, C₁-C₄haloalkyl, OR¹⁴, SR¹⁵, or NR¹⁶R¹⁷, COR¹³; or
R¹⁸ is C₁-C₆alkylene or C₂-C₆alkenylene linked to the phenyl or naphthyl ring of the carbazole moiety and forming a 5- or 6-membered saturated or unsaturated ring;
R¹⁹ is COOR¹⁴, CONR¹⁶R¹⁷, COR¹⁴; or
R¹⁹ has one of the meanings given for R¹⁶ and R¹⁷;
R²⁰ is COOR¹⁴, CONR¹⁶R¹⁷, or COR¹⁴; or
R²⁰ has one of the meanings given for R¹⁴;
R²³, R²⁴, R²⁵ and R²⁶ independently of each other are hydrogen, C₁-C₂₀alkyl, COR¹³, COOR¹⁴, OR¹⁴, or NO₂;
k is an integer 1 to 10;
m is 1 or 2;
n is integer 1 to 20;
X¹ is O, S, SO or SO₂;
X² is O, CO, S or a direct bond;
M is C₁-C₂₀alkylene which is unsubstituted or substituted by one or more substituents selected from the group consisting of halogen, OR¹⁴, SR¹⁵, NR¹⁶R¹⁷, CN, COOR¹⁴, CONR¹⁶R¹⁷, C₃-C₈cycloalkyl which is uninterrupted or interrupted by one or more O, S, CO or NR¹⁶, C₆-C₂₀aryl, C₃-C₂₀heteroaryl, C₆-C₂₀aroyl and C₃-C₂₀heteroarylcarbonyl, which C₆-C₂₀aryl, C₃-C₂₀heteroaryl, C₆-C₂₀aroyl or C₃-C₂₀heteroarylcarbonyl is unsubstituted or substituted by one or more halogen, phenyl, C₁-C₂₀alkylphenyl, C₁-C₈alkoxyphenyl, C₁-C₄haloalkyl, CN, NO₂, OR¹⁴, SR¹⁵, or NR¹⁶R¹⁷; or
M is C₂-C₂₀alkylene interrupted by one or more O, S, NR¹⁶, CO, SO or SO₂, which C₁-C₂₀alkylene or C₂-C₂₀alkylene is unsubstituted or substituted by one or more substituents selected from the group consisting of C₃-C₈cycloalkyl, OH, SH, O(CO)R^{3a}, COOR¹⁴, CONR¹⁶R¹⁷, C₆-C₂₀aryl, C₃-C₂₀heteroaryl, C₆-C₂₀aroyl and C₃-C₂₀heteroarylcarbonyl, wherein C₆-C₂₀aryl, C₃-C₂₀heteroaryl, C₆-C₂₀aroyl or C₃-C₂₀heteroarylcarbonyl is unsubstituted or substituted by one or more halogen, C₁-C₈alkyl, OR¹⁴, SR¹⁵, or NR¹⁶R¹⁷; or
M is C₂-C₁₂alkenylene or C₃-C₂₀cycloalkylene, which C₂-C₁₂alkenylene or C₃-C₂₀cycloalkylene is uninterrupted or interrupted by one or more O, S, CO, NR¹⁶ or COOR¹⁴; or
M is C₆-C₂₀arylene or C₃-C₂₀heteroarylene, which C₆-C₂₀arylene or C₃-C₂₀heteroarylene is unsubstituted or substituted by one or more halogen, C₁-C₂₀alkyl, C₁-C₄haloalkyl, phenyl, C₁-C₂₀alkylphenyl, C₁-C₈alkoxyphenyl, CN, NO₂, OR¹⁴, SR¹⁵, NR¹⁶R¹⁷, COOR¹⁴, COR^{3a} or SO₂R^{3a}; and
Y is a direct bond, O, S or NR¹⁸.

2. Compounds of the formula I according to claim 1, wherein the compounds are compounds of formula II: 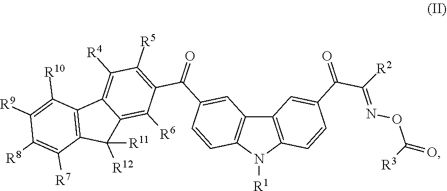
R¹ is C₁-C₂₀alkyl which is unsubstituted or substituted by one or more halogen, OR¹⁴, SR¹⁵, COOR¹⁴, NR¹⁶R¹⁷, CONR¹⁶R¹⁷, PO(OCₖH₂ₖ₊₁)₂; or
R¹ is C₂-C₂₀alkyl which is interrupted by one or more O, CO, S, C(O)O, OC(O), phenylene, naphthylene or NR¹⁸, wherein the interrupted C₁-C₂₀alkyl is unsubstituted or substituted by one or more halogen, OR¹⁴, SR¹⁵, COOR¹⁴ NR¹⁶R¹⁷, CONR¹⁶R¹⁷; or
R¹ is C₆-C₂₀aryl or C₃-C₂₀heteroaryl each of which is unsubstituted or substituted by one or more phenyl, halogen, C₁-C₄haloalkyl, CN, NO₂, OR¹⁴, SR¹⁵, NR¹⁶R¹⁷, 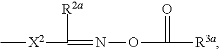
or by C₂-C₂₀alkyl which is interrupted by one or more O, S, or NR¹⁸, or each of which is substituted by one or more C₁-C₂₀alkyl which is unsubstituted or substituted by one or more halogen, COOR¹⁴, CONR¹⁶R¹⁷, phenyl, C₃-C₈cycloalkyl, C₃-C₂₀heteroaryl, C₆-C₂₀aryloxycarbonyl, C₃-C₂₀heteroaryloxycarbonyl, OR¹⁴, SR¹⁵ or NR¹⁶R¹⁷;
R² is hydrogen, C₁-C₂₀alkyl which is unsubstituted or substituted by one or more substituents halogen, OR¹⁴, SR¹⁵, NR¹⁶R¹⁷, COOR¹⁴, 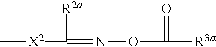
or C₃-C₈cycloalkyl which is uninterrupted or interrupted by one or more O; or
R² is C₂-C₂₀alkyl interrupted by one or more O, which interrupted C₂-C₂₀alkyl is unsubstituted or substituted by one or more C₃-C₈cycloalkyl, OH, O(CO)R^{3a} or COOR¹⁴; or
R² is C₂-C₁₂alkenyl, C₃-C₂₀cycloalkyl or C₆-C₂₀aryl, which C₆-C₂₀aryl or C₃-C₂₀heteroaryl is unsubstituted or substituted by one or more halogen, NO₂, OR¹⁴, SR¹⁵, COOR¹⁴, COR^{3a} or 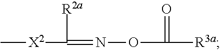
or
R^{2a} is hydrogen, COR^{3a}, COOR^{14a}, C₁-C₂₀alkyl which is unsubstituted or substituted by one or more halogen, OR^{14a}, SR^{15a} or C₃-C₈cycloalkyl which is uninterrupted or interrupted by one or more O;
R³ is C₁-C₂₀alkyl which is unsubstituted or substituted by one or more halogen, OR¹⁴, SR¹⁵, NR¹⁶R¹⁷, COOR¹⁴, C₃-C₈cycloalkyl or C₃-C₈cycloalkyl which is interrupted by one or more O; or
R³ is C₂-C₂₀alkyl interrupted by one or more O, which interrupted C₂-C₂₀alkyl is unsubstituted or substituted by one or more OH, O(CO)R^{3a} or COOR¹⁴; or
R³ is C₂-C₁₂alkenyl or C₃-C₂₀cycloalkyl; or
R³ is C₆-C₂₀aryl or C₃-C₂₀heteroaryl, each of which is unsubstituted or substituted by one or more halogen or C₁-C₂₀alkyl; or
R³ is C₁-C₂₀alkoxy, C₆-C₂₀aryloxy or C₃-C₂₀heteroaryloxy;
R^{3a} is hydrogen, C₁-C₂₀alkyl, C₂-C₂₀alkyl interrupted by one or more O, C₂-C₁₂alkenyl, C₃-C₂₀cycloalkyl, C₆-C₂₀aryl or C₃-C₂₀heteroaryl, which C₆-C₂₀aryl or C₃-C₂₀heteroaryl is unsubstituted or substituted by one or more halogen, C₁-C₂₀alkyl, OR^{14a}, SR^{15a}, NR^{16a}R^{17a}, COOR¹⁴³, (CO)—(C₁-C₈alkyl) or benzoyl; or
R^{3a} is C₆-C₂₀aryloxy or C₃-C₂₀heteroaryloxy, which C₆-C₂₀aryloxy or C₃-C₂₀heteroaryloxy is unsubstituted or substituted by benzoyl, or 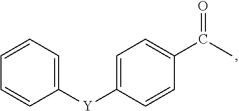
which benzoyl
or 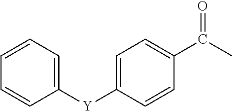
is unsubstituted or substituted by 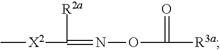
R⁴ to R¹² are defined as in claim 1;
R¹⁴ is hydrogen, COR^{3a}, C₁-C₂₀alkyl which is unsubstituted or substituted by one or more C₃-C₈cycloalkyl which is uninterrupted or interrupted by one or more O; or
R¹⁴ is C₂-C₂₀alkyl interrupted by one or more O, which interrupted C₂-C₂₀alkyl is unsubstituted or substituted by one or more C₃-C₈cycloalkyl, OH or COOR^{14a}; or
R¹⁴ is C₂-C₁₂alkenyl, C₃-C₂₀cycloalkyl or C₆-C₂₀aryl, which C₆-C₂₀aryl is unsubstituted or substituted by one or more halogen or C₁-C₂₀alkyl; or
R^{14a} is C₁-C₂₀alkyl;
R¹⁵ is hydrogen, C₁-C₂₀alkyl which is unsubstituted or substituted by one or more OR^{14a}, COOR^{14a}, C₃-C₈cycloalkyl or C₆-C₂₀aryl; or
R¹⁵ is C₂-C₂₀alkyl interrupted by one or more O, which interrupted C₂-C₂₀alkyl is unsubstituted or substituted by one or more OH or COOR^{14a}; or
R¹⁵ is C₂-C₁₂alkenyl or C₃-C₂₀cycloalkyl; or
R¹⁵ is C₆-C₂₀aryl which is unsubstituted or substituted by one or more halogen or C₂-C₂₀alkyl which is uninterrupted or interrupted by one or more O;
R^{15a} is C₁-C₂₀alkyl;
R¹⁶ and R¹⁷ independently of each other are hydrogen, C₁-C₂₀alkyl which is unsubstituted or substituted by one or more halogen, OR^{14a}, SR^{15a}, COOR^{14a}, C₃-C₈cycloalkyl or C₆-C₂₀aryl; or
R¹⁶ and R¹⁷ independently of each other are C₂-C₂₀alkyl interrupted by one or more O, which interrupted C₂-C₂₀alkyl is unsubstituted or substituted by OH or SH; or
R¹⁶ and R¹⁷ independently of each other are C₂-C₁₂alkenyl, C₃-C₂₀cycloalkyl or C₆-C₂₀aryl; or
R¹⁶ and R¹⁷ together with the N-atom to which they are attached form a 5- or 6-membered saturated or unsaturated ring which is uninterrupted or interrupted by O, S or NR^{16a};
R^{16a} and R^{17a} independently of each other are hydrogen or C₁-C₂₀alkyl; and
X² is CO or a direct bond.

3. Compounds of the formula I according to claim 1 or 21, wherein the compounds are compounds of formula III: 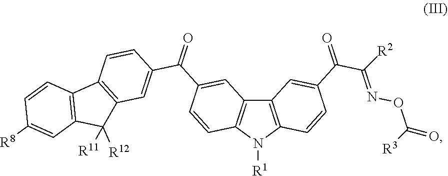
wherein
R¹ is C₁-C₂₀alkyl which is unsubstituted or substituted by NR¹⁶R¹⁷; or
R¹ is C₂-C₂₀alkyl which is interrupted by one or more C(O)O, OC(O) or phenylene, wherein the interrupted C₁-C₂₀alkyl is unsubstituted or substituted by NR¹⁶R¹⁷; or
R¹ is phenyl or thienyl each of which is unsubstituted or substituted by 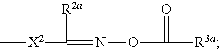
R² is C₁-C₂₀alkyl which is unsubstituted or substituted by halogen, SR¹⁵, NR¹⁶R¹⁷, COOR¹⁴ or C₃-C₈cycloalkyl; or
R² is C₆-C₂₀aryl which is unsubstituted or substituted by C₁-C₂₀alkyl;
R^{2a} is C₁-C₂₀alkyl or C₆-C₂₀aryl;
R^{2a} is C₆-C₂₀aryl which is unsubstituted or substituted by C₁-C₂₀alkyl; or
R³ is C₁-C₂₀alkyl, C₆-C₂₀aryl or C₁-C₂₀alkoxy;
R^{3a} is C₁-C₂₀alkyl or C₆-C₂₀aryl;
R⁸ is H, NO₂, or COR¹³, wherein
R¹¹ and R¹² independently of each other are hydrogen or C₁-C₂₀alkyl,
R¹³ is phenyl, naphthyl or thienyl which is unsubstituted and substituted by phenyl, C₁-C₂₀alkyl, OR¹⁴, SR¹⁵, or NR¹⁶R¹⁷, wherein
R¹⁴ is C₁-C₂₀alkyl or R¹⁴ together with one of the carbon atoms of R² forms a 5- or 6-membered;
R¹⁵ is C₆-C₂₀aryl; and
R¹⁶ and R¹⁷ independently of each other are O(CO)R^{3a}, COR^{3a} or C₁-C₂₀alkyl.

4. Compounds of the formula I according to claim 1, wherein the compounds are compounds of formula IV: 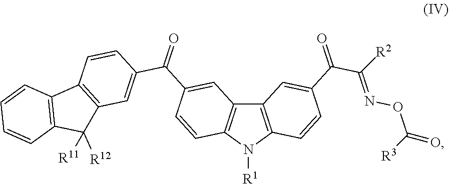
wherein
R¹ to R³, R¹¹ and R¹² are defined as in claim 1.

5. A photopolymerizable composition comprising
(a) at least one ethylenically unsaturated photopolymerizable compound and
(b) as photoinitiator, at least one compound of the formula I as defined in claim 1.

6. A photopolymerizable composition according to claim 5, wherein the component (a) is a resin obtained by the reaction of a saturated or unsaturated polybasic acid anhydride with a product of the reaction of an epoxy resin and an unsaturated monocarboxylic acid.

7. A photopolymerizable composition according to claim 5, additionally to the photoinitiator (b) comprising at least one further photoinitiator (c), and/or other additives (d).

8. A photopolymerizable composition according to claim 5 as further additive (d) comprising a pigment, or a mixture of pigments, or a dye, or a mixture of dyes, or a mixture of one or more pigments with one or more dyes.

9. A photopolymerizable composition according to claim 8 as further additive (d) comprising a dispersant or a mixture of dispersants.

10. A photopolymerizable composition according to claim 5, comprising 0.05 to 25% by weight of the photoinitiator (b), or the photoinitiators (b) and (c), based on the weight of the composition.

11. A photopolymerizable composition according to claim 5 as further additive (d) comprising a photo sensitizer, in particular a compound selected from the group consisting of benzophenone, benzophenone derivatives, thioxanthone, thioxanthone derivatives, anthraquinone, anthraquinone derivatives, coumarin and coumarine derivatives.

12. A photopolymerizable composition according to claim 5 additionally comprising a binder polymer (e), in particular a copolymer of methacrylate and methacrylic acid.

13. A process for the preparation of a compound of the formula I according to claim 1 by reacting the corresponding oxime compound with an acyl halide of the formula I′ or an anhydride of the formula I″ 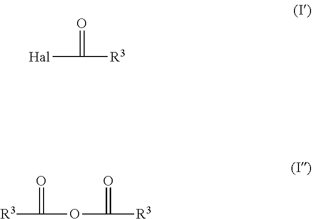
wherein Hal is a halogen, in particular CI, and R³ is as defined in claim 1, in the presence of a base or a mixture of bases.

14. A process for the photopolymerization of compounds containing ethylenically unsaturated double bonds, which comprises irradiating a composition according to claim 5 with electromagnetic radiation in the range from 150 to 600 nm, or with electron beam or with X-rays.

15. A process according to claim 14 for producing pigmented and nonpigmented paints and varnishes, powder coatings, printing inks, printing plates, adhesives, pressure sensitive adhesives, dental compositions, gel coats, photoresists for electronics, electroplating resists, etch resists, both liquid and dry films, solder resists, resists to manufacture color filters for a variety of display applications, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, colour proofing systems, glass fibre cable coatings, screen printing stencils, for producing three-dimensional objects by means of stereolithography, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board.

16. Coated substrate which is coated on at least one surface with a composition according to claim 5.

17. Process for the photographic production of relief images, in which a coated substrate according to claim 16 is subjected to imagewise exposure and then the unexposed portions are removed with a developer.

18. A color filter prepared by providing red, green and blue picture elements and a black matrix, all comprising a photosensitive resin and a pigment on a transparent substrate and providing a transparent electrode either on the surface of the substrate or on the surface of the color filter layer, wherein said photosensitive resin comprises a polyfunctional acrylate monomer, an organic polymer binder and a photopolymerization initiator of formula I as defined in claim 1.

19. Use of a compound of the formula (I) as defined in claim 1 for the photopolymerization of a composition comprising at least one ethylenically unsaturated photopolymerizable compound.

20. Compounds of the formula (IA) 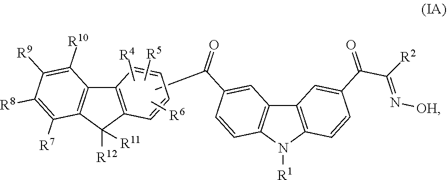
wherein
R¹, R² and R⁴ to R¹² are defined as in claim 1.
